SEQUENCE LISTING (56 sequences)

SEQ ID NO: 1 - protein, 24 aa, Artificial Sequence
  P. aeruginosa N′ terminal signal peptide
MKLKRLMAALTFVAAGVGAASAVA

SEQ ID NO: 2 - protein, 15 aa, Artificial Sequence
  P. aeruginosa N′ loop
AIDPALPEYQKASGV

SEQ ID NO: 3 - protein, 8 aa, Artificial Sequence
  P. aeruginosa the conserved strand 1
SGNLSSVG

SEQ ID NO: 4 - protein, 23 aa, Artificial Sequence
  C.perfringens N′ terminal signal peptide
MFKKRLIAIIGTIFIGATAMVGC

SEQ ID NO: 5 - protein, 10 aa, Artificial Sequence
  C.perfringens N′ loop
NSGGSEAKST

SEQ ID NO: 6 - protein, 8 aa, Artificial Sequence
  C.perfringens the conserved strand 1
NSVSISGS

SEQ ID NO: 7 - protein, 23 aa, Artificial Sequence
  S.peneumoina N′ terminal signal peptide
MKKRKKLALSLIAFWLTACLVGC

SEQ ID NO: 8 - protein, 7 aa, Artificial Sequence
  S.peneumoina N′ loop
ASWIDRG

SEQ ID NO: 9 - protein, 8 aa, Artificial Sequence
  S.peneumoina the conserved strand 1
ESITAVGS

SEQ ID NO: 10 - protein, 22 aa, Artificial Sequence
  L.brevis N′ terminal signal peptide
MSKKRWVQGLVLVILMALGIYA

SEQ ID NO: 11 - protein, 10 aa, Artificial Sequence
  L.brevis N′ loop
YQTREVSHAG

SEQ ID NO: 12 - protein, 8 aa, Artificial Sequence
  L.brevis the conserved strand 1
ESITAVGS

SEQ ID NO: 13 - protein, 20 aa, Artificial Sequence
  M.tuberculosis N′ terminal signal peptide
MKIRLHTLLAVLTAAPLLLA

SEQ ID NO: 14 - protein, 29 aa, Artificial Sequence
  M.tuberculosis N′ loop
AAGCGSKPPSGSPETGAGAGTVATTPASS

SEQ ID NO: 15 - protein, 8 aa, Artificial Sequence
  M. tuberculosis the conserved strand 1
PVTLAETG

SEQ ID NO: 16 - protein, 25 aa, Artificial Sequence
  Y.pestis N′ terminal signal peptide
MKLMRTTVASIVAATLSMTAVSAFA

SEQ ID NO: 17 - protein, 4 aa, Artificial Sequence
  Y.pestis N′ loop
FAEA

SEQ ID NO: 18 - protein, 8 aa, Artificial Sequence
  Y.pestis the conserved strand 1
SLTGAGAT

SEQ ID NO: 19 - protein, 25 aa, Artificial Sequence
  E.coli N′ terminal signal peptide
MKVMRTTVATVVAATLSMSAFSVFA

SEQ ID NO: 20 - protein, 8 aa, Artificial Sequence
  E.coli the conserved strand 1
EASLTGAG

SEQ ID NO: 21 - protein, 26 aa, Artificial Sequence
  V. cholerae N′ terminal signal peptide
MIRMALAAVCALLFSITTMTPFVQAS

SEQ ID NO: 22 - protein, 8 aa, Artificial Sequence
  V.cholerae the conserved strand 1
EITISGST

SEQ ID NO: 23 - protein, 10 aa, Artificial Sequence
  Inhibitory peptide
XAIDPALPEX

SEQ ID NO: 24 - protein, 9 aa, Artificial Sequence
  Inhibitory peptide
AIDPALPEX

SEQ ID NO: 25 - protein, 17 aa, Artificial Sequence
  Peptide-1
AIDPALPEYQKASGVSG

SEQ ID NO: 26 - protein, 13 aa, Artificial Sequence
  Peptide-2
AIDPALPEYQKAS

SEQ ID NO: 27 - protein, 8 aa, Artificial Sequence
  Peptide-3
AIDPALPE

SEQ ID NO: 28 - protein, 5 aa, Artificial Sequence
  Peptide-4
PEYQK

SEQ ID NO: 29 - protein, 4 aa, Artificial Sequence
  Peptide-5
EYQK

SEQ ID NO: 30 - protein, 9 aa, Artificial Sequence
  Peptide-6
YQKASGVSG

SEQ ID NO: 31 - DNA, 51 bp, Artificial Sequence
  Primer PstSUpF01-GWB1
ggggacaagtttgtacaaaaaagcaggctcacaattgccctggaaactacc

SEQ ID NO: 32 - DNA, 42 bp, Artificial Sequence
  Primer PstSUpR01
tacaggcccagttccttgatcgccggccgccatcaaacgctt

SEQ ID NO: 33 - DNA, 18 bp, Artificial Sequence
  Primer PstSDownF01
ggcgatcaaggaactggg

SEQ ID NO: 34 - DNA, 47 bp, Artificial Sequence
  Primer PstSDownR01-GWB2
ggggaccactttgtacaagaaagctgggtacgaccagcacgtaccag

SEQ ID NO: 35 - DNA, 47 bp, Artificial Sequence
  Primer 1320
agaggaattctaaggaggaataacatatgaaactcaagcgtttgatg

SEQ ID NO: 36 - DNA, 64 bp, Artificial Sequence
  Primer 1322
agagaagcttttagtgatggtgatggtgatgctccagggcggcggccaggcccagttccttgat

SEQ ID NO: 37 - DNA, 32 bp, Artificial Sequence
  Primer Ser96US
aacctgggcccgatggaacgcaagatgaagga

SEQ ID NO: 38 - DNA, 33 bp, Artificial Sequence
  Primer Ser96DS
gtccttcatcttgcgttccatcgggcccaggtt

SEQ ID NO: 39 - DNA, 31 bp, Artificial Sequence
  Primer 1325
agagacatgttctttcctgcgttatcccctg

SEQ ID NO: 40 - DNA, 17 bp, Artificial Sequence
  Primer 1326
cgggcaacctgtcgagc

SEQ ID NO: 41 - DNA, 34 bp, Artificial Sequence
  Primer 1327
gctcgacaggttgcccgcgcggctaccgcggaag

SEQ ID NO: 42 - DNA, 34 bp, Artificial Sequence; identical to SEQ ID NO: 41
  Primer 010-YO

SEQ ID NO: 43 - DNA, 31 bp, Artificial Sequence
  Primer 004-YO
gcgcaagcttcaggcccagttccttgatcgc

SEQ ID NO: 44 - DNA, 33 bp, Artificial Sequence
  Primer 257-YO
aacctgggcccgatggaacgcaagatgaaggac

SEQ ID NO: 45 - DNA, 33 bp, Artificial Sequence; identical to SEQ ID NO: 38
  Primer 258-YO

SEQ ID NO: 46 - DNA, 29 bp, Artificial Sequence
  Primer 264-YO
atatgaattcggtgtcgggcaacctgtcg

SEQ ID NO: 47 - protein, 323 aa, Artificial Sequence
  P.aeruginosa  PstS - accession number NP_254056.1
MKLKRLMAALTFVAAGVGAASAVAAIDPALPEYQKASGVSGNLSSVGSDTLANLMTMWAEEYKRLYPNVNIQIQAAGSSTAPPALTEGTANLGPMSRKMKDVELQAFEQKYGYKPTAVPVAVDALAIFVHKDNPIKGLTMQQVDAIFSATRLCGSKQDVKTWGDLGLTGDWAKKPVQLFGRNSVSGTYGYFKEEALCKGDFRPNVNEQPGSASVVQSVSQSLNGIGYSGIGYKTASVKTVALAKKEGAAFVEDNEQNALNGTYPLSRFLYVYVNKAPNKPLDPLEAQFLKLVLSKTGQQVVVKDGYIPLPAKVAEKAIKELGL

SEQ ID NO: 48 - protein, 14 aa, Artificial Sequence
  P. aeruginosa Residues 25 to 38 of P. aeruginosa PstS
AIDPALPEYQKASG

SEQ ID NO: 49 - protein, 16 aa, Artificial Sequence
  P. aeruginosa, Residues 245 to 260 of P. aeruginosa PstS
KEGAAFVEDNEQNALN

SEQ ID NO: 50 - protein, 38 aa, Artificial Sequence
  P. aeruginosa, Residues 1 to 38 of P. aeruginosa PstS
MKLKRLMAALTFVAAGVGAASAVAAIDPALPEYQKASG

SEQ ID NO: 51 - protein, 10 aa, Artificial Sequence
  Inhibitory peptide
XAXXXXLXXX

SEQ ID NO: 52 - protein, 323 aa, Artificial Sequence
  The S96E PstS mutant
MKLKRLMAALTFVAAGVGAASAVAAIDPALPEYQKASGVSGNLSSVGSDTLANLMTMWAEEYKRLYPNVNIQIQAAGSSTAPPALTEGTANLGPMERKMKDVELQAFEQKYGYKPTAVPVAVDALAIFVHKDNPIKGLTMQQVDAIFSATRLCGSKQDVKTWGDLGLTGDWAKKPVQLFGRNSVSGTYGYFKEEALCKGDFRPNVNEQPGSASVVQSVSQSLNGIGYSGIGYKTASVKTVALAKKEGAAFVEDNEQNALNGTYPLSRFLYVYVNKAPNKPLDPLEAQFLKLVLSKTGQQVVVKDGYIPLPAKVAEKAIKELGL

SEQ ID NO: 53 - protein, 310 aa, Artificial Sequence
  The N-terminus deleted PstS construct
MKLKRLMAALTFVAAGVGAASAVAGVSGNLSSVGSDTLANLMTMWAEEYKRLYPNVNIQIQAAGSSTAPPALTEGTANLGPMSRKMKDVELQAFEQKYGYKPTAVPVAVDALAIFVHKDNPIKGLTMQQVDAIFSATRLCGSKQDVKTWGDLGLTGDWAKKPVQLFGRNSVSGTYGYFKEEALCKGDFRPNVNEQPGSASVVQSVSQSLNGIGYSGIGYKTASVKTVALAKKEGAAFVEDNEQNALNGTYPLSRFLYVYVNKAPNKPLDPLEAQFLKLVLSKTGQQVVVKDGYIPLPAKVAEKAIKELGL

SEQ ID NO: 54 - DNA, 21 bp, Artificial Sequence
  Primer 1785
taaaagcttggcactggccgt

SEQ ID NO: 55 - DNA, 21 bp, Artificial Sequence
  Primer 1786
accgctggctttctgatattc

SEQ ID NO: 56 - protein, 8 aa, Artificial Sequence
  Peptide-3 D-enantiomer
AIDPALPE

(19) United States
(12) Patent Application Publication
(10) Pub. No.: US 2017/0064966 A1
(43) Pub. Date: Mar. 9, 2017

(54) ANTIMICROBIAL COMPOSITIONS AND METHODS AND USES THEREOF

(71) Applicant: BAR ILAN UNIVERSITY, Ramat Gan (IL)

(72) Inventors: Yarden OPATOWSKY, Raanana (IL); Ehud BANIN, Tel Aviv (IL); Avi NEZNANSKY, Givat Koah (IL); Inna BLUS-KADOSH, Kfar Saba (IL); Gal YERUSHALMI, Givat Shmuel (IL)

(73) Assignee: BAR ILAN UNIVERSITY, Ramat Gan (IL)

(21) Appl. No.: 15/258,425

(22) Filed: Sep. 7, 2016

Related U.S. Application Data
(60) Provisional application No. 62/215,196, filed on Sep. 8, 2015.

(51) Int. Cl. A01N 63/02; C07K 14/21

(52) U.S. Cl. CPC A01N 63/02; C07K 14/21; A61K 38/00

(57) ABSTRACT

The invention relates to inhibitors of a bacterial biofilm formation that target the N′ loop extension of the periplasmic subunit of a bacterial Phosphate Specific Transfer system (PstS), specifically, of P. aeruginosa. The inhibitors of the invention may be either derived from the N′ loop extension of PstS or directed against the N′ loop extension. The invention further provides compositions and methods using said inhibitors in inhibiting biofilm formation and in treating pathologic conditions associated therewith.

### FIELD OF THE INVENTION

The present invention pertains to the field of antimicrobial and antibiofilm therapies. More specifically, the present invention relates to inhibitors targeting specific component of the conserved bacterial inorganic Phosphate Specific Transport (Pst) system, and provides compositions, methods and uses thereof in interfering with the formation of bacterial biofilms.

### BACKGROUND REFERENCES

1. Zaborina, O., Holbrook, C., Chen, Y. M., Long, J., Zaborin, A., Morozova, I., Fernandez, H., Wang, Y. M., Turner, J. R., and Alverdy, J. C. (2008) Structure-function aspects of PstS in multi-drug-resistant Pseudomonas aeruginosa. Plos Pathogens 4
2. Blus-Kadosh, I., Zilka, A., Yerushalmi, G., and Banin, E. (2013) The effect of pstS and phoB on quorum sensing and swarming motility in Pseudomonas aeruginosa. PLoS One 8, e74444
3. Neznansky, A., and Opatowsky, Y. (2014) Expression, purification and crystallization of the phosphate-binding PstS protein from Pseudomonas aeruginosa. Acta Crystallogr Sect F Struct Biol Cryst Commun 70, 5
4. Berntsson, R. P., Smits, S. H., Schmitt, L., Slotboom, D. J., and Poolman, B. A structural classification of substrate-binding proteins. FEBS Lett 584, 2606-2617
5. Bastonero, S., Le Priol, Y., Armand, M., Bernard, C. S., Reynaud-Gaubert, M., Olive, D., Parzy, D., de Bentzmann, S., Capo, C., and Mege, J. L. (2009) New microbicidal functions of tracheal glands: defective anti-infectious response to Pseudomonas aeruginosa in cystic fibrosis. PLoS One 4, e5357
6. Lewenza, S., Falsafi, R. K., Winsor, G., Gooderham, W. J., McPhee, J. B., Brinkman, F. S., and Hancock, R. E. (2005) Construction of a mini-Tn5-1uxCDABE mutant library in Pseudomonas aeruginosa PAO1: a tool for identifying differentially regulated genes. Genome Res 15, 583-589
7. Fischer, R. J., Oehmcke, S., Meyer, U., Mix, M., Schwarz, K., Fiedler, T., and Bahl, H. (2006) Transcription of the pst operon of Clostridium acetobutylicum is dependent on phosphate concentration and pH. J Bacteriol 188, 5469-5478
8. Madhusudhan, K. T., McLaughlin, R., Komori, N., and Matsumoto, H. (2003) Identification of a major protein upon phosphate starvation of Pseudomonas aeruginosa PAO1. J Basic Microbiol 43, 36-46
9. Holloway, B. W., Krishnapillai, V., and Morgan, A. F. (1979) Chromosomal genetics of Pseudomonas. Microbiological reviews 43, 73-102
10. Blus-Kadosh, I., Zilka, A., Yerushalmi, G., and Banin, E. (2013) The Effect of pstS and phoB on Quorum Sensing and Swarming Motility in Pseudomonas aeruginosa. PloS one 8, e74444
11. Woodcock, D. M., Crowther, P. J., Doherty, J., Jefferson, S., DeCruz, E., Noyer-Weidner, M., Smith, S. S., Michael, M. Z., and Graham, M. W. (1989) Quantitative evaluation of Escherichia coli host strains for tolerance to cytosine methylation in plasmid and phage recombinants. Nucleic acids research 17, 3469-3478
12. Schweizer, H. P. (1991) Escherichia-Pseudomonas shuttle vectors derived from pUC18/19. Gene 97, 109-121
13. Rybtke, M. T., Borlee, B. R., Murakami, K., Irie, Y., Hentzer, M., Nielsen, T. E., Givskov, M., Parsek, M. R., and Tolker-Nielsen, T. (2012) Fluorescence-based reporter for gauging cyclic di-GMP levels in Pseudomonas aeruginosa. Applied and environmental microbiology 78, 5060-5069
14. Schweizer, H. P., and Hoang, T. T. (1995) An improved system for gene replacement and xylE fusion analysis in Pseudomonas aeruginosa. Gene 158, 15-22
15. Hou, C. I., Gronlund, A. F., and Campbell, J. J. (1966) Influence of phosphate starvation on cultures of Pseudomonas aeruginosa. Journal of bacteriology 92, 851-855
16. Weiss Nielsen, M., Sternberg, C., Molin, S., and Regenberg, B. (2011) Pseudomonas aeruginosa and Saccharomyces cerevisiae biofilm in flow cells. Journal of visualized experiments:JoVE
17. Hollenstein, K., Frei, D. C., and Locher, K. P. (2007) Structure of an ABC transporter in complex with its binding protein. Nature 446, 213-216
18. Wang, Z., Choudhary, A., Ledvina, P. S., and Quiocho, F. A. (1994) Fine tuning the specificity of the periplasmic phosphate transport receptor. Site-directed mutagenesis, ligand binding, and crystallographic studies. J Biol Chem 269, 25091-25094

### BACKGROUND OF THE INVENTION

Pseudomonas aeruginosa (PA) is an opportunistic Gram-negative pathogen that causes infection and sepsis, particularly individuals with compromised natural defenses. PA is further a primary cause of nosocomial infections. A key element in PA pathogenicity is its ability to form biofilms that withstand eradication by antibiotics and the immune system. One of the key factors that control formation of biofilms is phosphate signaling. Phosphate is a vital nutrient that participates in many cellular functions such as nucleotide metabolism, divalent ions absorption, growth, stress processes and virulence regulation.

PA has been shown to possess two phosphate transport mechanisms: the ‘Phosphate Inorganic Transport’ Pit system, and the ‘Phosphate Specific Transport’ Pst system, which is an ATP-binding cassette (ABC) transporter. While the Pit system is a one-proton one-Pi symporter and has a low Km affinity toward phosphate, the Pst system has a ten-fold stronger affinity to Pi and, like Pit, is also docked on the bacterial inner membrane. Expression of the Pst system is induced under sub-millimolar phosphate concentrations and, thereby, complements the Pit proton symporter, which is active under higher phosphate concentrations.

PstS, the periplasmic subunit of the pst transporter, captures free phosphate and brings it to the pst transmembrane permease. PstS was initially discovered as a periplasmic phosphate binding protein in Escherichia coli, and only later identified as a component of the conserved bacterial Pst system. More recently, PstS has been implicated in biofilm formation characteristic of PA strains in general and of multi drug resistant strains in particular. Specifically it has been shown that PA strains secret PstS that is used for the construction of fibers, described as “appendages”, which further facilitate PA adhesion to epithelial lining in vitro and in vivo (1). In a previous work the present inventors have demonstrated that PstS is vital for phosphate uptake in PA and that its deletion induces a hyper-surface motility (swarming) response on plates irrespective of phosphate levels. Hyper-swarming can be similarly induced in wild-type PA by growth under phosphate-limiting conditions, thus supporting the role of PstS in linking surface motility and phosphate uptake (2). PstS was recently crystallized by the inventors (3) classified to cluster D-M of the substrate binding protein (SBP) superfamily according to the classification presented by Bernts son et al. (4).

Antimicrobial resistance is one of the most serious health threats. Multidrug resistance of PA is of particular concern, as PA is one of the common causes of healthcare-associated infections including pneumonia, bloodstream infection, urinary tract infections and surgical site infections. First, PA is intrinsically resistant to a large number of antibiotics and can acquire resistance to many others, making treatment difficult. Second, the propensity of PA to form biofilms further protects it from antibiotics and from the host immune system. Currently, the mainstay therapy for PA infection is based on antimicrobials (or antibiotics), including two-drug combination therapy such as an antipseudomonal beta-lactam with an aminoglycoside.

Because antibiotic resistance occurs as part of a natural evolution process, it can be significantly slowed but not stopped. Therefore, new antibiotics will always be needed to keep up with resistant bacteria as well as new diagnostic tests to track the development of resistance. The number of new antibiotics developed and approved has steadily decreased in the past three decades, leaving fewer options to treat resistant bacteria. There is therefore an urgent need for alternative approaches for preventing biofilm formation and treating biofilm-related infections.

### SUMMARY OF THE INVENTION

In a first aspect, the invention relates to an inhibitor of a bacterial biofilm formation comprising at least one of: (a) at least one amino acid sequence derived from the N′ loop extension of the periplasmic subunit of a bacterial Phosphate Specific Transfer system (PstS), or of any fragment thereof; and (b) at least one compound that specifically binds to said N′ loop extension of PstS.

In a further aspect, the invention provides an isolated and purified peptide comprising the amino acid sequence of the N′ loop extension of P. aeruginosa PstS and any derivatives and fragments thereof.

In a further aspect, the invention relates to an isolated and purified nucleic acid sequence encoding the N′ loop extension of P. aeruginosa PstS or any fragment thereof.

A further aspect of the invention relates to a composition comprising at least one inhibitor of a bacterial biofilm formation, as described by the invention and optionally further comprises at least one pharmaceutically acceptable carriers, excipients, auxiliaries, and/or diluents.

A further aspect of the invention relates to a method for inhibiting, reducing or eliminating bacterial biofilm formation in at least one of a subject, a surface and a substance, the method comprising administering to said subject, or contacting, applying or dispensing to said surface or substance an effective amount of at least one inhibitor of a bacterial biofilm formation according to the invention or any composition comprising the same.

Still further aspect relates to a method for treating, preventing, ameliorating, reducing or delaying the onset of an infectious clinical condition in a subject in need thereof using the inhibitors and compositions described by the invention.

The invention further provides a screening method for an antimicrobial compound that inhibits, reduces or eliminates bacterial biofilm formation.

These and further aspects of the invention will become apparent as the description proceeds.

### BRIEF DESCRIPTION OF THE DRAWINGS

FIGS. 1A-1C. Crystal structure and topography of PA PstS

FIG. 1A shows a ribbon diagram of PA PstS crystal structure. Domain I is colored in light gray, domain II in dark gray, and the N′ loop in black; PO₄ is depicted as balls.

FIG. 1B shows a secondary structure diagram of PA PstS, segregated into two domains. Strands are depicted as arrows, helixes as cylinders, and 3₁₀ helixes as rectangles.

FIG. 1C illustrates structural homology of PstS orthologs. In the top panel: sequence alignment of the N′ terminal signal peptide in italics, N′ loop in bold, and the conserved strand 1 in regular font. In the bottom panel: homology tree representing the structural similarity of PA PstS to its orthologs. The ProCKSI server was used for comparison of the eight bacterial PstS structures using an “all against all” comparison mode. For similarity model generation, DaliLite and universal similarity metric (USM) alignments were used. PDB codes: 4GD5—C. perfringens, 4LAT—S. pneumonia, 4ECF—L. brevis, 1PC3—M. tuberculosis, 2Z22—Y. pestis, 1IXH—E. coli, 1TWY—V. cholera.

Amino acid sequences of the fragments of the different PstS orthologs are numbered as follows: P. aeruginosa N′ terminal signal peptide, N′ loop and the conserved strand 1 are denoted by SEQ ID NOs. 1, 2 and 3, respectively; C. perfringens N′ terminal signal peptide, N′ loop and the conserved strand 1 are denoted by SEQ ID NOs. 4, 5 and 6, respectively; S. pneumonia N′ terminal signal peptide, N′ loop and the conserved strand 1 are denoted by SEQ ID NOs. 7, 8 and 9, respectively, L. brevis N′ terminal signal peptide, N′ loop and the conserved strand 1 are denoted by SEQ ID NOs. 10, 11 and 12, respectively; M. tuberculosis N′ terminal signal peptide, N′ loop and the conserved strand 1 are denoted by SEQ ID NOs. 13, 14 and 15, respectively; Y. pestis N′ terminal signal peptide, N′ loop and the conserved strand 1 are denoted by SEQ ID NOs. 16, 17 and 18, respectively; E. coli N′ terminal signal peptide and the conserved strand 1 are denoted by SEQ ID NOs. 19 and 20, respectively; V. cholera N′ terminal signal peptide and the conserved strand 1 are denoted by SEQ ID NOs. 21 and 22, respectively.

FIGS. 2A-2B. Comparison between PstS crystal structures

FIG. 2A shows the crystal structure of form-1 (light gray) and form-2 (dark gray) PA PstS are superimposed and are virtually identical. The two regions that are missing from the structure of form-2, i.e., the N′ loop and helix 8, are encircled.

FIG. 2B shows the crystal structures of form-1 (light gray) superimposed onto the E. coli PstS crystal structure (PDB code 1IXH, in dark gray).

FIGS. 3A-3D. Phosphate binding by PA PstS

FIG. 3A is 2Fo-Fc electron density map contoured at 1.5 sigma level showing a close-up view of PA PstS PO₄ binding. The backbone, side chains, and PO₄ are represented as sticks. Serine 96 and Arginine 181 are indicated.

FIG. 3B is ligplot 2D diagram of the PO₄ interactions with PA PstS polypeptide. Residues from domain II are underscored and hydrogen bonds are depicted as dashed lines.

FIG. 3C shows binding curves of P³²-labeled phosphate to wild-type PstS (squares) compared to the S96E pstS (circles) and the delN′ pstS (triangles) mutants. Binding constants (indicated) were calculated by nonlinear regression curve fitting with the GraphPad Prism software using the following equation: Y=Bmax*X/(Kd+X).

FIG. 3D shows levels of alkaline phosphatase (AP) activity, which is related to phosphate uptake, in PA pstS wild-type (PAO1) and mutant strains. AP activity was assayed in PAO1, ΔpstS, and ΔpstS complemented with either wild-type pstS, S96E pstS, or delN′ pstS. Results were normalized and represent mean+standard deviation for three independent experiments. Each sample was performed in triplicate. Asterisks represent the significant rise in AP activity compared to the WT (p<0.05, Student's t-test).

FIGS. 4A-4C. Wild-type and mutant PA PstS have a similar elution profile

Figures show elution profiles from size-exclusion chromatography of the wild-type and mutant PA PstS. The wild-type PstS (FIG. 4A) and mutants S96E (FIG. 4B) and delN′ (FIG. 4C) were expressed in E. coli and isolated by consecutive metal chelate and ion exchange chromatography before being analyzed using a Superdex 200 10/300 gel filtration column (GE Healthcare). The elution profile and volume are consistent with monomeric protein arrangements (arrows indicate estimated molecular mass, kDa) and indicate well-folded proteins.

FIGS. 5A-5J. Influence of pstS deletion and mutations on PA swarming motility

Figures show images of swarming motility assays, wherein PAO1 carrying an empty vector was grown on swarming plates containing M9 (20 mM phosphate; +Pi; FIG. 5A) or phosphate-depleted M9 (0.2 mM phosphate; −Pi; FIG. 5B) and compared to PA ΔpstS that was carrying an empty vector (FIGS. 5C, 5D) or complemented with wild-type pstS (FIGS. 5E, 5F), S96E pstS (FIGS. 5G, 5H), or delN′ pstS (FIGS. 5I, 5J).

FIGS. 6A-6C. Intra- and inter-molecular interactions of the PA PstS N′ loop

FIG. 6A illustrates intra-molecular interactions of the N′ loop of PA PstS. The N′ loop is depicted by stick representation where the N′ terminus is indicated, and the rest of the protein is represented as an electrostatic surface.

FIG. 6B illustrates crystal contacts of PA PstS in the P2₁2₁2₁ lattice. The N′ loop are encircled. There are additional crystal contacts that are not represented here.

FIG. 6C shows ligplot 2D diagram of the intra-molecular interactions of the N′ loop, including the multiple atomic interactions in the Ala25-Tyr33 range of the N′ loop.

FIG. 7. Influence of pstS deletion and mutations on PA biofilm formation

Figure shows biofilm forming capacity (expressed as biovolume) of PAO1 carrying an empty vector and ΔpstS carrying either an empty vector, a complementation plasmid with wild type pstS, pstS with S96E pstS, or pstS without the N′ loop (DelN′). Bacteria were grown for 72 h in a flow chamber biofilm system. Results shown represent mean+standard deviation of four independent experiments. Results were normalized to those of PAO1/vector. Asterisks represent the significant attenuation in biofilm formation compared to ΔpstS/pstS (P<0.05, Tukey's post hoc test).

FIG. 8. PstS is required for biofilm formation

Figure shows confocal microscope images of the wild-type (W.T) and ΔpstS deletion mutant. Bacteria were grown in a flow cell biofilm reactor, using 1% tryptic soy broth as growth media at 37° C. for 72 h, and stained with Syto-9.

FIG. 9. Ectopic expression of the N′-loop of PstS interferes with biofilm formation

Figure shows biofilm forming capacity of PA carrying an empty vector, a vector expressing the N′-loop (NTerm) and ΔpstS carrying an empty vector. Bacteria were grown for 72 h in a flow chamber biofilm system. Results represent means+/−sd of 4 independent experiments. Results were normalized to those of PA/vector and analyzed as in FIG. 7.

FIGS. 10A-10C. N′-loop peptides inhibit biofilm formation

FIG. 10A shows N′-loop sequences and boundaries of synthesized peptides 1-6, as denoted by SEQ ID NO. 25-30, respectively.

FIG. 10B shows biofilm forming capacity of PA exposed to 0.1 millimolar of peptides 1-6 (as denoted by SEQ ID NO. 25-30, respectively). Figure demonstrates the effect of various peptides on inhibition of biofilm formation, the most effective peptide being peptide 3.

FIG. 10C shows biofilm forming capacity of PA exposed to 0.1 millimolar of a modified peptide 3, where the terminal amino acids are D enantiomers, and therefore less susceptible to degradation by proteases. The peptide-3 enantiomer is denoted by SEQ ID NO. 56.

FIGS. 11A-11B. N′-loop peptide enantiomer inhibits biofilm formation in clinical strains of PA

FIG. 11A shows biofilm forming capacity (expressed as biovolume) of different PA strains, specifically, PAO1 strain that express genomic GFP and the PA14 and clinical isolates DK2 that express Plasmidic GFP, exposed to 0.1 millimolar of peptide-3 enantiomer (as denoted by SEQ ID NO. 56). The microscope images were analyzed by Imaris software. Results are normalized to each strain without the addition of peptide. The experiment was done in triplicates.

FIG. 11B figure shows confocal microscope images of the different PA strains, specifically, PAO1 strain and PA14 and the clinical isolates DK2, treated with the D-enantiomer peptide 3. The bacteria were grown on a 1μ-Slide for 48 hours at 37° C. and pictures were taken using SP8 confocal HyD microscope (Leica).

### DETAILED DESCRIPTION OF THE INVENTION

This invention stems from presently disclosed studies using X-ray crystallography structural analyses and functional assays, which have led to characterization the PstS subunit of the PA Pst phosphate transporter and its surprising role in PA biofilm formation. Specifically, these studies revealed the unique underpinnings of PstS phosphate binding and have led to identification of an unusual 15-residue N′ loop extension and its specific function.

Structure-based experiments showed that PstS-mediated phosphate uptake and biofilm formation are in fact two distinct functions, which further could be distinguished from each other using mutagenesis. Specifically, a point mutation that abrogated phosphate binding did not eliminate biofilm formation and, conversely, truncation of the N′ loop diminished the ability of PA to form biofilms but had no effect on phosphate binding and uptake. This places PstS at a junction that separately controls phosphate sensing, uptake and the ultra-structure organization of bacteria.

Present findings are, in fact, surprising in view of the conventional notion that bacterial ability to form biofilms and phosphate signaling are inter-related and that latter controls biofilm formation. The present studies have demonstrated that the dual activities attributed to PstS, biofilm formation and phosphate uptake, are independent and mapped to different sites of this protein. In this sense, PstS being the periplasmic component of the Pst phosphate transporter is also a structural protein. This unique duality in PstS function intrinsically integrates biofilm formation and nutritional cues, even though phosphate binding per se is not required for PstS biofilm activity.

Yet another important realization stemming from present findings is that the N′ loop of PA PstS is crucial for the buildup of PA biofilm and that this particular PstS feature may represent a novel antibiofilm target. This realization has been ultimately reduced to practice in showing that artificial short peptide fragments mapping to a specific region within the N′ loop of PA are capable of inhibiting biofilm formation in a dose-specific manner.

More specifically, in previous studies the inventors observed that PstS deletion in PA results in decreased phosphate uptake, and also activate the hyper-swarming response (2). Having established that in PA PstS exhibits two activities, the phosphate response and biofilm formation, the inventors hypothesized that there are three possible mechanisms for this phenomenon: (1) that in the course of bacterial colonization, there are changes in phosphate levels that are detected by PstS and serve as signals for biofilm development; (2) that extracellular PstS serves as a building block in the construction of adhesion appendages, regardless of phosphate binding and transport properties, and (3) a combination of two, whereby PstS is involved in structural aspects of biofilm construction and also in signaling the response to phosphate limitation.

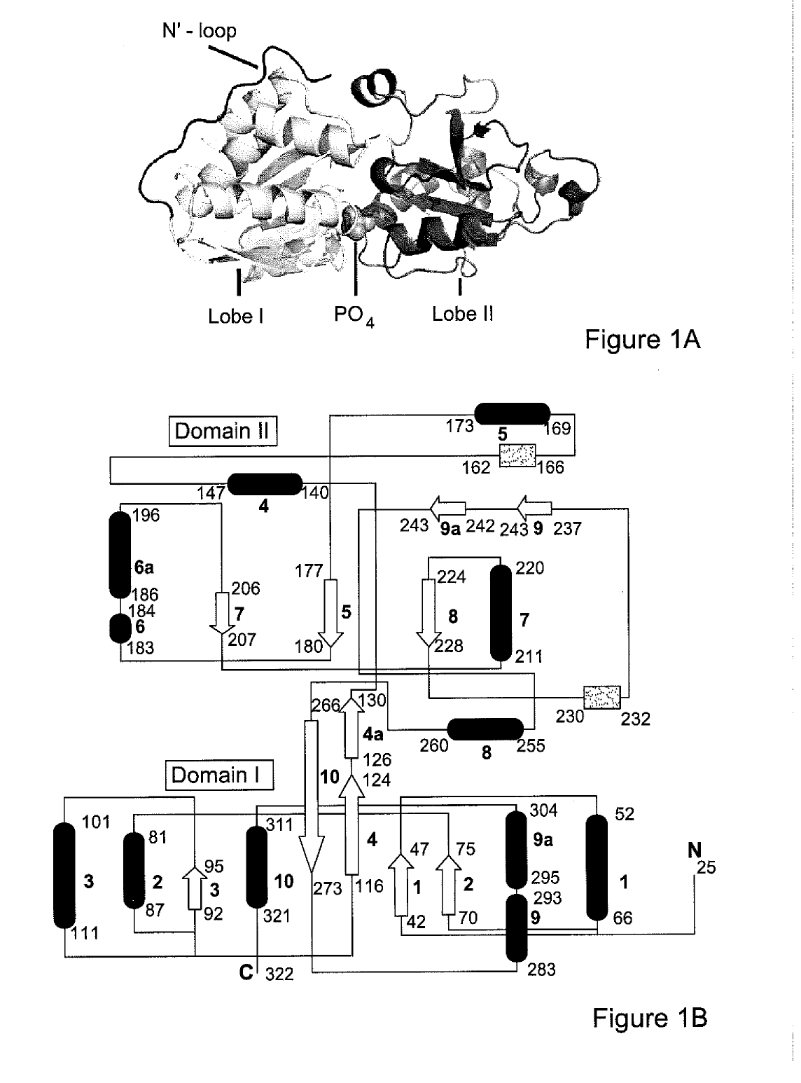

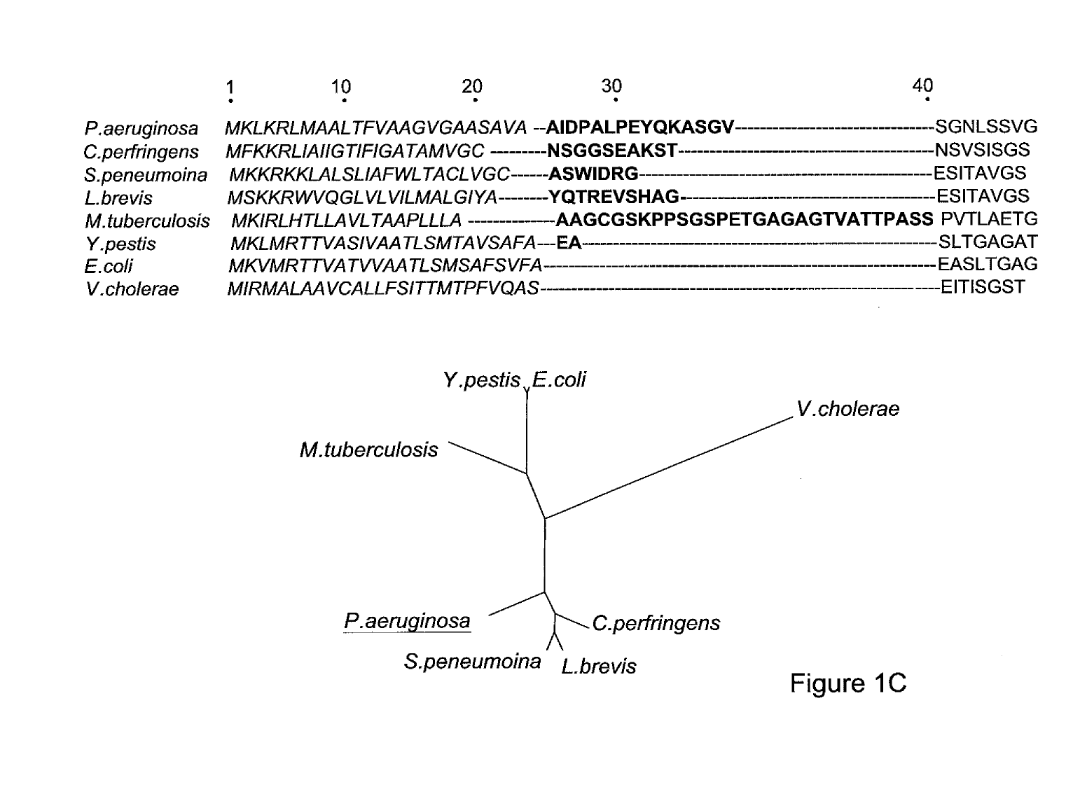

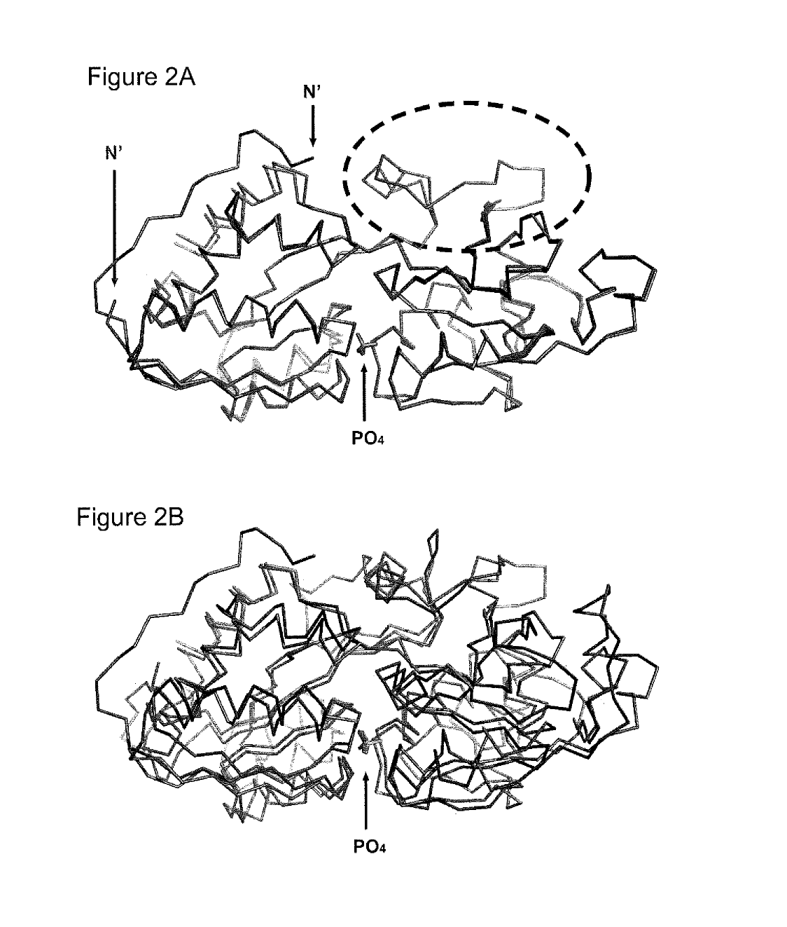

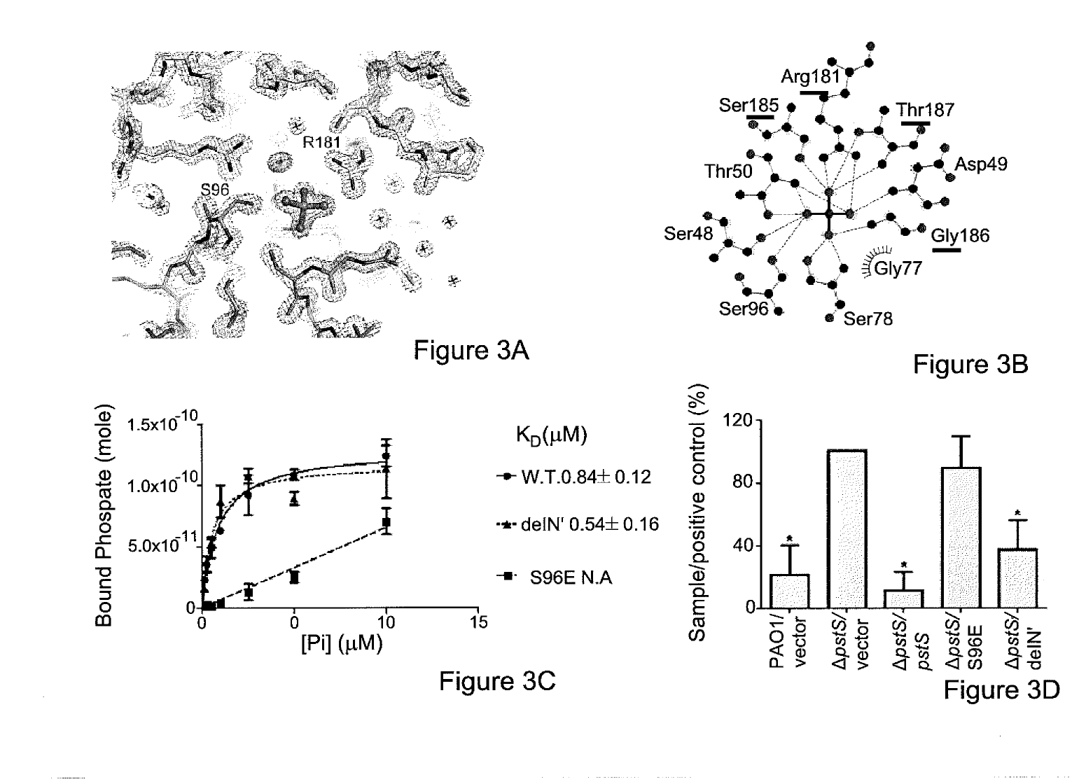

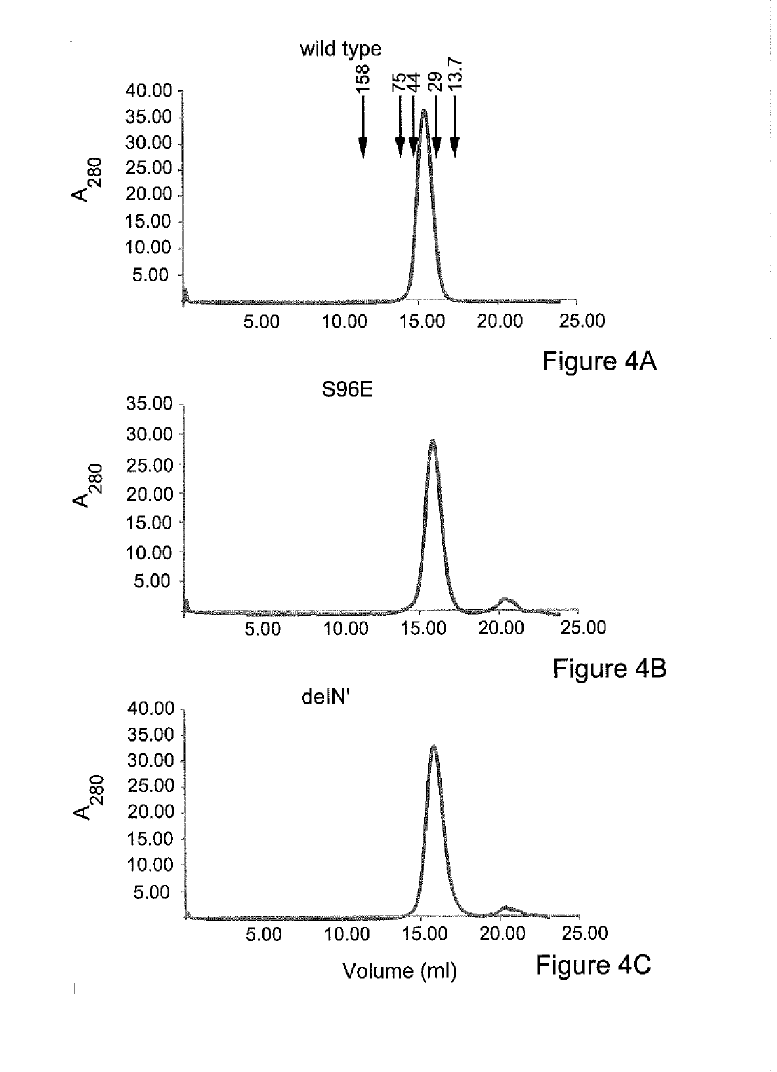

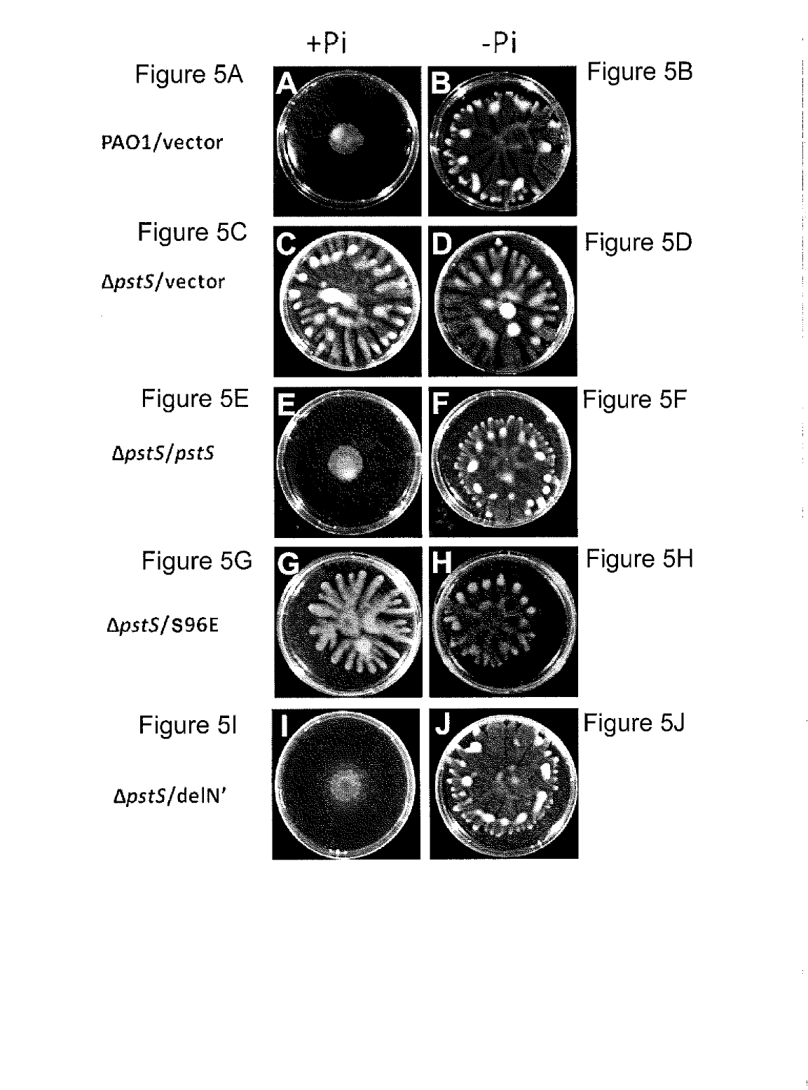

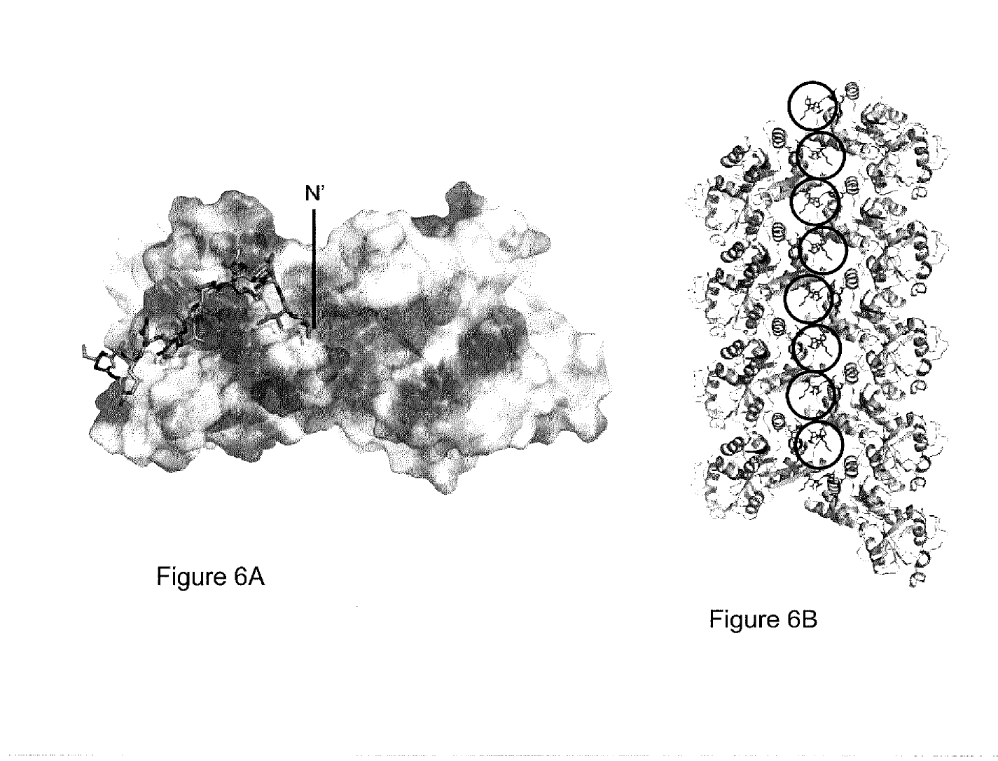

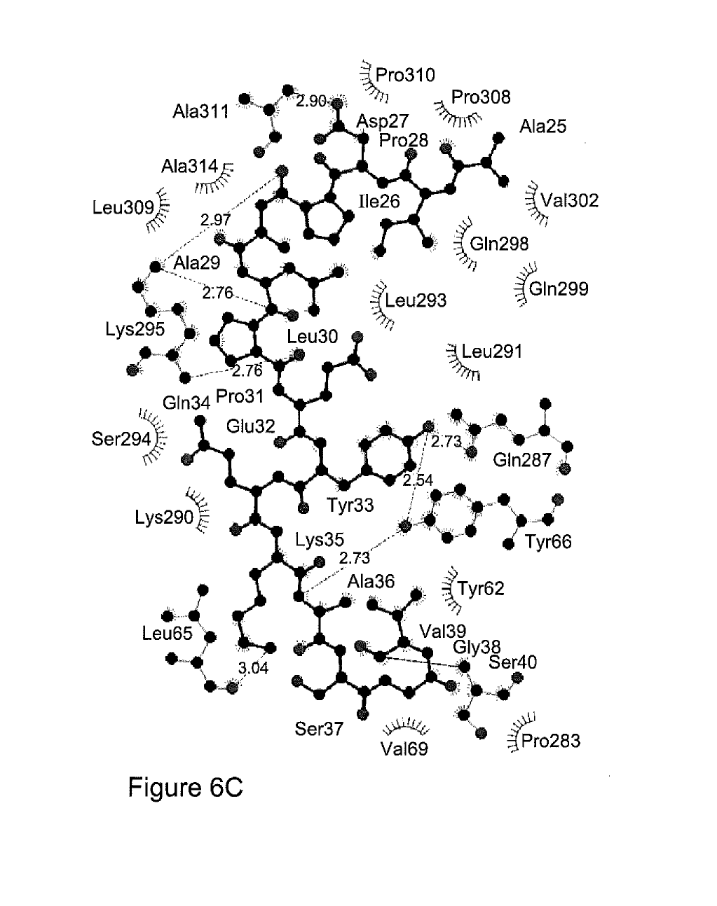

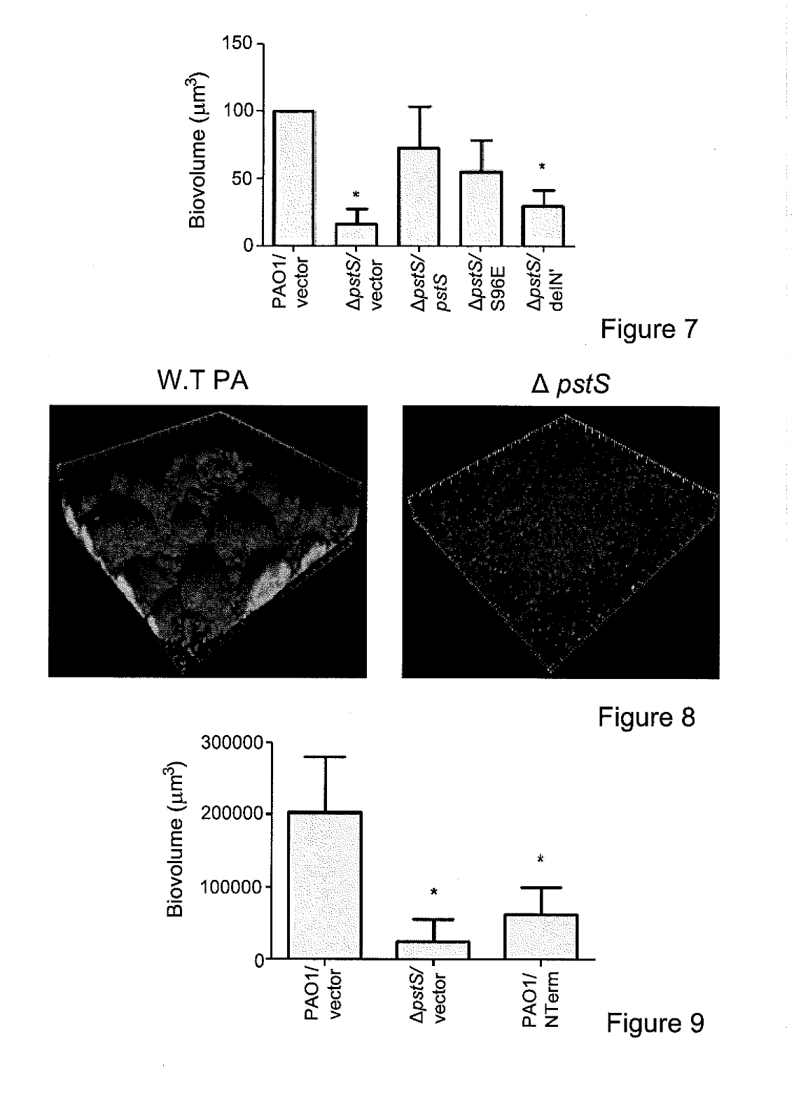

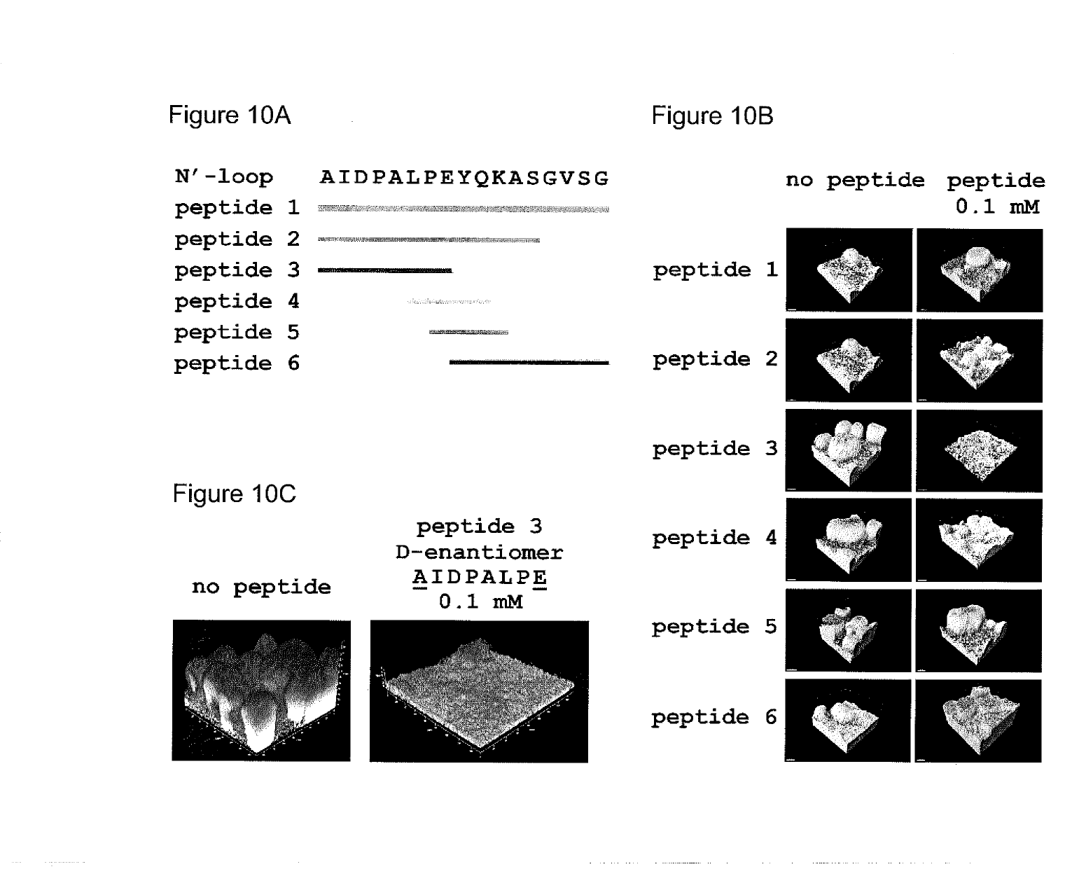

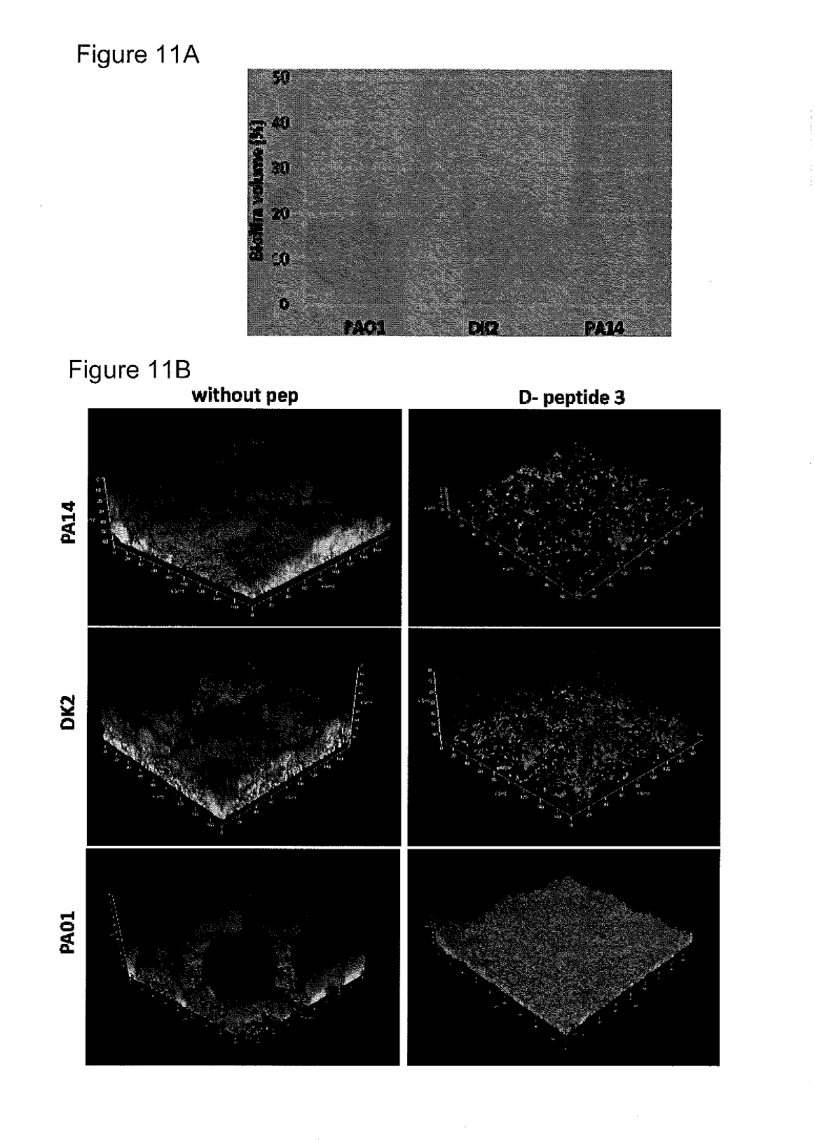

To empirically differentiate between these possibilities, the inventors sought to create PA PstS mutants defective in either phosphate binding or biofilm formation. To which end, they determined and analyzed the crystal structure of PA PstS in order to identify phosphate-binding residues that could be mutated, such that the resulting mutant would be incapable of binding phosphate yet would maintain overall structural integrity (FIGS. 1A-1C and FIGS. 2A-2B). To abrogate phosphate binding, they replaced one of the PO₄-interacting residues, Serine 96, with a glutamate, which—based on analysis of the crystal structure—would pose steric and electrostatic interference to PO₄ binding. Indeed, the resulting S96E-mutant PstS protein possessed no or very weak PO₄ binding and, accordingly, S96E-mutant PstS bacteria exhibit lower phosphate uptake (FIG. 3D) and the same hyper-swarming phenotype under phosphate-rich conditions as the PstS knockout strain (FIGS. 5G-5H). These findings confirmed that S96E-mutant PstS bacteria are incompetent in mediating phosphate transport into the bacterial cytoplasm. The structural integrity of the S96E protein, as validated by analytical size-exclusion chromatography showing very similar elution profiles for the wild-type and S96E PstS proteins, was consistent with monomeric protein arrangements (FIG. 4). This gel filtration assay and the observation that similar amounts of wild-type and S96E PstS proteins were delivered into the periplasm in the E. coli expression system, served as strong support for the premise that the mutant protein is structurally intact.

Further, having generated the S96E PstS mutant that was defective in phosphate binding and phosphate uptake yet structurally intact, the inventors investigated if it was still able to mediate biofilm formation. Most notably, they found that it was capable of generating biomass that was fairly similar to the wild-type PstS-complement strain (FIG. 7). Based on these results, the inventors concluded that the role of PstS in PA biofilm formation does not require the phosphate binding and transport activity of PstS. Moreover, relying on the notion that substrate binding proteins (SBPs) exist in an open-closed equilibrium in the absence of bound ligand, the inventors hypothesized that PstS's role in biofilm formation does not depend on a specific conformation, rather on other structural properties of the protein.

Further, the inventors investigated whether the biofilm activity of PstS is necessary for phosphate uptake. To that end, they sought to create a PstS mutant that would be defective in its ability to facilitate biofilm formation, while retaining phosphate binding and transport capabilities. They hypothesized that either amino or carboxy-terminal extensions would mediate intermolecular interactions between secreted proteins of biofilm-forming bacteria, thus produced a mutant PstS with an N′ loop truncation. This delN′ mutant was deficient in biofilm formation, similar to the ΔpstS knockout mutant (FIG. 7). In the same manner as for the S96E mutant, the structural integrity of PstS delN′ was confirmed by its periplasmic expression levels and size-exclusion-chromatography elution profile (FIG. 4). With regard to phosphate-dependent activities, the delN′ mutant exhibited a similar dissociation constant with PO₄, similar phosphate uptake (FIGS. 3C-3D), and similar swarming pattern (FIGS. 5I-5J) to wild type PstS. Taken together, these results support the finding that the N′ loop of PA PstS plays a structural role in the buildup of PA biofilm, and that this activity is not dependent on phosphate binding or uptake.

Ultimately in a series of further experiments the inventors showed that the biofilm formation in PA can be reduced or controlled by targeting the N′-loop of PA PstS. More specifically, they showed that PA biofilm formation can be dramatically reduced by ectopic expression of a vector constitutively expressing the PstS N′-loop along with the native signal peptide, required for periplasmic targeting (FIG. 9). Furthermore, they produced synthetic peptides having N′-loop derived sequences and demonstrated that peptides comprising the first eight amino acids of the N′-loop or any fragments thereof had specific dose-dependent effect in inhibiting the rate of bacterial biofilm formation (FIG. 10). Moreover, a D-enantiomer derivative of said peptide exhibited a marked inhibitory effect on biofilm formation. These results highlight the potential to inhibit or compete with the N′-loop as an anti-biofilm strategy.

Thus, in a first aspect, the invention relates to an inhibitor of a bacterial biofilm formation comprising at least one of:

(a) at least one amino acid sequence derived from the N′ loop extension of the periplasmic subunit of a bacterial Phosphate Specific Transfer system (PstS), any orthologs, or of any fragment thereof, or any nucleic acid sequence encoding the same; and (b) at least one compound that specifically binds to said N′ loop extension of PstS.

The term ‘bacterial biofilm’ is used herein in the sense of the IUPAC (International Union of Pure and Applied Chemistry) definition of this term, namely an aggregate of microorganisms, in this case bacteria, in which cells that are frequently embedded within a self-produced matrix of extracellular polymeric substance (EPS) adhere to each other and/or to a surface. This definition encompasses biofilms that adhere to biological or non-biological surfaces. Further, a biofilm is a fixed system that can be adapted internally to environmental conditions by its inhabitants.

The self-produced matrix of EPS (also referred to as slime) produced by bacteria is a polymeric conglomeration generally composed of extracellular biopolymers in various structural forms. More specifically, the bacterial EPS is a complex mixture consisting of polysaccharides, as well as proteins, nucleic acids and lipids and substances (HS, i.e. components of the Natural Organic Matter (NOM). EPS make up the intercellular space of microbial aggregates and form the structure and architecture of the biofilm matrix. The key functions of EPS comprise the mediation of the initial attachment of cells to different substrata and protection against environmental stress and dehydration.

Thus, bacterial biofilms represent a significant mode of bacterial growth, which is protective and allows survival in hostile environments. For example, biofilm growth has been considered to confer bacterial resistance to disinfections or to host-mediated immune response. Bacteria form a biofilm in response to many factors, including cellular recognition of specific or non-specific attachment sites on a surface, nutritional cues, or exposure to sub-inhibitory concentrations of antibiotics. When a cell switches to the biofilm mode of growth, it undergoes a phenotypic shift wherein large suites of genes are differentially regulated.

From a general point of view, bacterial biofilm formation (or biofilm development) has been divided into several key steps, including attachment, microcolony formation, biofilm maturation and dispersion. Until present, different components and molecules, including flagella, type IV pili, DNA and exopolysaccharides have been implicated in various steps of this process. Further, there are several genetic regulation mechanisms implicated in biofilm regulation, such as quorum sensing and the novel secondary messenger cyclic-di-GMP. Although it is yet to be determined in which step the N′ loop extension of PstS is implicated, the presently provided evidence of its surprising role in this process biofilm formation form basis for a novel strategy to inhibit the formation of bacterial biofilm, and thereby to inhibit or impair bacterial growth and/or infection, or bacterial virulence.

In this connection, under the term ‘virulence’ is meant the MeSH definition of this term, i.e. the degree of pathogenicity (or an ability to cause disease) within a group or species of microorganisms, as indicated by case fatality rates and/or the ability of the microorganism to invade the tissues of the host.

Further in this connection, it should be understood that by inhibiting bacterial biofilm formation is meant reducing, attenuating, eliminating, deferring, decreasing or impairing the capacity to form biofilms. In some specific and non-limiting examples, inhibition of biofilm formation may be evaluated or revealed in measurements of a biovolume using flow chamber biofilm system (FIG. 7 and FIG. 10B) or microscope images (FIG. 8). In certain embodiments, the inhibition by the inhibitors of the invention as described herein above, may be an inhibition, reduction, elimination, attenuation, retardation, decline, prevention or decrease of at least about 5%-99.9999%, about 10%-90%, about 15%-85%, about 20%-80%, about 25%-75%, about 30%-70%, about 35%-65%, about 40%-60% or about 45%-55%, and more specifically may be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, of the biofilm formation in the absence of any of the inhibitors of the invention. In yet some further embodiments, reduction and inhibition of biofilm formation may be in log terms, in the range of 2 to 6, specifically, 2, 3, 4, 5, 6 log. More specifically, 3-4 log reduction when compared to biofilm formation in the absence of the inhibitors of the invention.

As indicated above, an inhibitor of biofilm formation according to the invention may be an inhibitor that targets the N′ loop extension of the PstS protein. Such inhibitor may be a compound that derived from the N′ loop extension, specifically, a peptide, or alternatively, a compound that specifically recognizes and binds the N′ loop extension of the PstS protein. According to some embodiments, the N′ loop extension of PstS is of a Gram negative or a Gram positive bacteria PstS. In some specific embodiments, N′ loop extension forms a random coil secondary structure.

The term ‘bacteria’ (in singular a ‘bacterium’) in this context refers to any type of a single celled microbe. Herein the terms ‘bacterium’ and ‘microbe’ are interchangeable. This term encompasses herein bacteria belonging to general classes according to their basic shapes, namely spherical (cocci), rod (bacilli), spiral (spirilla), comma (vibrios) or corkscrew (spirochaetes), as well as bacteria that exist as single cells, in pairs, chains or clusters.

In specific embodiments, the term ‘bacteria’ specifically refers to Gram positive or Gram negative types of bacteria. The Gram-positive bacteria can be recognized as retaining the crystal violet stain used in the Gram staining method of bacterial differentiation, and therefore appear to be purple-colored under a microscope. The Gram-negative bacteria do not retain the crystal violet, making positive identification possible. In other words, the term ‘bacteria’ applies herein to bacteria with a thicker peptidoglycan layer in the cell wall outside the cell membrane (Gram-positive), and to bacteria with a thin peptidoglycan layer of their cell wall that is sandwiched between an inner cytoplasmic cell membrane and a bacterial outer membrane (Gram-negative). This term further applies to some bacteria, such as Deinococcus, which stain Gram-positive due to the presence of a thick peptidoglycan layer, but also possess an outer cell membrane, and thus suggested as intermediates in the transition between monoderm (Gram-positive) and diderm (Gram-negative) bacteria.

Specifically relevant to the present context are Gram-positive bacteria that can cause disease in animals and humans. Gram-positive bacteria are the cause of more than 50% of all bloodstream infections. There is an increased frequency and widespread dissemination of staphylococcal clones, for example, that are resistant to all β-lactam drugs. Infections caused by multidrug-resistant Gram-positive bacteria represent a major public health burden, in terms of morbidity and mortality and increased expenditure on patient management, and infection control measures. Staphylococcus aureus and Enterococcus spp. are established pathogens in the hospital environment, and their frequent multidrug resistance complicates therapy.

Among Gram-negative bacteria that are relevant to the present context may include, for example, most of the bacteria normally found in the gastrointestinal tract (GI) and further gonococci responsible for venereal disease, and meningococci—for bacterial meningitis. Bacteria responsible for cholera and bubonic plague are also Gram-negative. Gram-negative bacteria can be resistant to multiple drugs and increasingly become resistant to most of the available antibiotics. Of particular relevance to the present invention is a Gram-negative bacterium Pseudomonas aeruginosa spp., which was related to a number of diseases in animals and humans, including among others pneumonia, GI, urinary tract and skin infections, and septic shock.

According to the present invention, the presence of an N′ loop extension structure in the periplasmic component of the Pst ABC (ATP Binding Cassette) phosphate transporter of bacteria is a necessary feature to confer biofilm formation. Thus the presently proposed strategy for preventing or inhibiting biofilm formation is rooted in antagonizing this structure by introducing competing or binding reagents or compounds, or biological systems producing thereof. From a broader perspective, the presently proposed approach provides an alternative and an independent line of attack on microbial virulence and multi-drug resistance.

The present inventors have demonstrated several features of N′ loop extension structure (may be also referred to as N′-loop, N-loop or N loop, structure and further have shown how it can be identified in various bacterial strains. More specifically, the N′ loop extension structure may be identified by crystallization of the entire PstS protein under sodium malonate conditions with a P2₁2₁2₁ space group, whereby PstS will form a characteristic structure containing four PstS copies (form-1) comprising the N′ loop extension (FIG. 2A). This structure is exclusively characteristic to PstS form-1 crystals produced under the above conditions, and is absent in other PstS forms, for example form-2 C222₁ crystals. In this connection it should be noted that PstS can be isolated in high quantities from growth media and bacterial outer surfaces (1, 5), and that higher PstS expression levels can be induced under phosphate-limiting conditions (6, 7, 8).

Further, protein structure comparisons between known PstS comprising the N′ loop extension and other PstS orthologs (available at the Protein Data Base (PDB) for example), using r.m.s.d. score (root-mean-square-deviation of atomic positions), will facilitate identification of additional bacterial PstS with an analogous N′ loop extension (FIG. 1C bottom panel). The present inventors have shown how this method may be applied to identify differences in the structure of PstS of PA and E. coli (FIG. 2B), and further to surmise that the E. coli PstS, unlike the PA PstS, is devoid of the N′ loop extension, despite both of them being Gram-negative bacteria. On which basis, the inventors concluded that, apart from PA, the N′-loop extension seemed to be a common feature among PstSs of Gram-positive bacteria.

Further, as has been presently demonstrated, sequence alignments could be informative for identification of N′ loop primary sequence in other bacterial PstS orthologs, as the N′ loop extension maps downstream to the conserved N-terminal signal peptide and upstream to conserved strand 1 (FIG. 1C top panel).

It should be appreciated that in specific embodiments of the invention, the N′ loop extension of PstS is of a PstS of a Gram negative or Gram positive bacteria, and said N′ loop extension forms a random coil secondary structure. The random coil is a class of conformations characterized in an absence of regular secondary structure.

It should be further appreciated that the present invention further encompasses the N′ loop extension of PstS as well as partial or fragmental sequence of the N′ loop extension, which as presently demonstrated, can be used as effective inhibitor/s of bacterial biofilm formation (FIGS. 10A-10C). These inhibitor/s are derived from the amino acid sequence of the N′ loop extension of PstS. Basing on this example, it is conceived that fragments comprising 8 amino acids or more of the N′ loop extension of PstS can be effective inhibitors, and further fragment comprising at least 3 amino acids or more and up to at least 30 amino acids, derived from or partially derived from the PstS N′ loop extension or from any flanking sequences thereof, may be similarly effective inhibitors.

It is further contemplated that the presently proposed approach for inhibiting bacterial biofilm formation by antagonizing the activity of the N′ loop extension of PstS could be particularly applicable to PstS is of P. aureginosa. In one specific embodiment, said PstS is also denoted by accession number NP_254056.1. In more specific embodiments, the PstS comprise the amino acid sequence of SEQ ID NO. 47.

In some specific embodiments, the N′ loop extension comprises residues 25 to 39 of P. aeruginosa PstS, as denoted by SEQ ID NO. 2 or any derivative or fragment thereof.

As indicated above, specific embodiments of the invention relate to N′ loop extensions of PA PstS, however, it should be appreciated that the invention further encompass inhibitors based on other PstS orthologs. In some alternative and particular embodiments, the inhibitors of the invention may be based on compounds derived from the N′ loop extension of any PstS ortholog that has an N′ loop extension. The term ‘ortholog’ denotes a polypeptide or protein obtained from one species that is the functional counterpart of a polypeptide or protein from a different species. Sequence differences among orthologs are the result of speciation. Thus, in certain embodiments, the invention further provides an inhibitor derived from or directed against the N′ loop extension of other PstS orthologs. Non-limiting examples include PstS of any one of C. perfringens, S. phenumonia, L. brevis, M. tuberculosis and Y. pestis or any ortholog thereof. Some specific embodiments include but are not limited to the N′ loop extension of the C. perfringens PstS comprising the sequence NSGGSEAKST of residues 25-35, as denoted by SEQ ID NO. 5, the N′ loop extension of the S. pneumonia PstS comprising the sequence ASWIDRG of residues 25-31, as denoted by SEQ ID NO. 8, the N′ loop extension of the L. brevis PstS comprising the sequence YQTREVSHAG of residues 25-34, as denoted by SEQ ID NO. 11, the N′ loop extension of the M. tuberculosis PstS comprising the sequence AAGCGSKPPSGSPETGAGAGTVTTPASS of residues 25-53 as denoted by SEQ ID NO. 14 or the N′ loop extension of the Y. pestis PstS comprising the sequence EA of residues 25-26, as denoted by SEQ ID NO. 17. It should be noted that SEQ ID NO. 17 further contains additional N-terminal residues of the Y. pestis PstS, specifically, FAEA, however, in some embodiments, the EA residues may be used as the N-loop.

In some further embodiments, the inhibitor of the invention may be at least one isolated and purified peptide comprising the amino acid sequence Xaa₍ₙ₎₋Ala¹-Xaa²-Xaa³-Xaa⁴-Xaa⁵-Leu⁶-Xaa⁷-Xaa⁸-Xaa₍ₙ₎ as denoted by SEQ ID NO. 51 or any fragment/s, enantiomer/s or derivative/s thereof, wherein Xaa is any amino acid and n is zero or an integer of from 1 to 10, and wherein

X¹, may be Ala or any hydrophobic, acidic or polar amino acid selected from Glu, Tyr and Asn;
X², may be Ile or an acidic or positively charged amino acid selected from His, Thr, Asp, Arg and Lys;
X³, may be Asp an acidic or positively charged amino acid selected from His, Thr, Ile, Arg and Lys;
X⁴, may be Pro or any other amino acid residue having a cyclic side chain;
X⁵, may be Ala or an hydrophobic amino acid;
X⁶, may be Leu or any other non-polar amino acid residue;
X⁷, may be Pro or any other amino acid residue having a cyclic side chain;
X⁸, may be Glu or any acidic or positively charged amino acid.

Still further, in some embodiments, the inhibitor/s of the invention may be at least one peptide derived from the N′ loop extension of the PA PstS. Such peptide may comprise according to certain embodiments of the invention, at least part of the amino acid sequence of the N′ loop extension of the PA PstS protein. It should be appreciated that the peptide may be extended either in the N′ or C′ termini thereof, or both, as described for example in the peptide comprising the amino acid sequence of Xaa₍ₙ₎₋Ala-Ile-Asp-Pro-Ala-Leu-Pro-Glu-Xaa₍ₙ₎ as denoted by SEQ ID NO. 23 or any fragment or derivative thereof, wherein Xaa is any amino acid and n is zero or an integer of from 1 to 10, specifically, 1, 2, 3, 4, 5, 6, 7, 8, 9, 10.

In some specific embodiments, the inhibitor of the invention may be at least one isolated and purified peptide comprising the amino acid sequence Ala-Ile-Asp-Pro-Ala-Leu-Pro-Glu as denoted by SEQ ID NO. 27 or any fragment/s, enantiomers, or derivative/s thereof. It should be noted that in certain embodiments, this peptide is also referred to herein as Peptide-3.

It should be appreciated that derivatives of any of the peptides of the invention include also any enantiomers thereof. More specifically, such enantiomers may comprise at least one amino acid residue in D-form. In yet some further embodiments, the peptides of the invention may comprise at least two, at least three, at least four, at least five, at least six, at least seven, at least eight, at least nine, at least ten or more amino-acid residues in the D-form.

In some further embodiments, at least one amino acid residue of an enantiomer of a peptide comprising the amino acid sequence as denoted by SEQ ID NO. 27, may be a D-enantiomer.

Of particular interest is an enantiomer peptide of SEQ ID NO. 27, where the N-terminal Ala and the C terminal Glu of the peptide are D-enantiomers. In more specific embodiments the peptide comprises the amino acid sequence as denoted by SEQ ID NO. 56.

In some further embodiments, the inhibitor may be at least one isolated and purified peptide comprising the amino acid sequence Ala-Ile-Asp-Pro-Ala-Leu-Pro-Glu-Xaa₍ₙ₎ as denoted by SEQ ID NO. 24 or any fragment/s, enantiomer/s or derivative/s thereof, wherein Xaa is any amino acid and n is zero or an integer of from 1 to 10.

In yet further specific embodiments, the inhibitor may be at least one isolated and purified peptide comprising the amino acid sequence Ala-Ile-Asp-Pro-Ala-Leu-Pro-Glu-Tyr-Gln-Lys-Ala-Ser as denoted by SEQ ID NO. 26 or any fragment or derivatives thereof. It should be noted that in certain embodiments, this peptide is also referred to herein as Peptide-2.

Some further embodiments relate to the inhibitor of the invention that may be at least one isolated and purified peptide comprising the amino acid sequence Ala-Ile-Asp-Pro-Ala-Leu-Pro-Glu-Tyr-Gln-Lys-Ala-Ser-Gly-Val-Ser-Gly as denoted by SEQ ID NO. 25 or any fragment or derivatives thereof. It should be noted that in certain embodiments, this peptide is also referred to herein as Peptide-1.

Still further, the inhibitor of the invention may be at least one isolated and purified peptide comprising the amino acid sequence Pro-Glu-Tyr-Gln-Lys as denoted by SEQ ID NO. 28 or any fragment or derivatives thereof. It should be noted that in certain embodiments, this peptide is also referred to herein as Peptide-4.

In further embodiments the inhibitor of the invention may be at least one isolated and purified peptide comprising the amino acid sequence Glu-Tyr-Gln-Lys, as denoted by SEQ ID NO. 29 or any fragment or derivatives thereof. It should be noted that in certain embodiments, this peptide is also referred to herein as Peptide-5.

Still further, the inhibitor of the invention may be at least one isolated and purified peptide comprising the amino acid sequence Tyr-Gln-Lys-Ala-Ser-Gly-Val-Ser-Gly as denoted by SEQ ID NO. 30 or any fragment or derivatives thereof. It should be noted that in certain embodiments, this peptide is also referred to herein as Peptide-6.

As shown in Example 7, using a construct that encodes residues 1-38 of PA PstS, the inventors showed that ectopic expression of the N terminal portion of PA PstS clearly inhibited biofilm formation. Thus, in certain specific embodiments, the invention further provide an inhibitor that comprises the amino acid sequence of residues 1-38 of PA PstS: MKLKRLMAALTFVAAGVGAASAVAAIDPALPEYQKASG, as denoted by SEQ ID NO. 50, or any derivative/s, fragment/s or enantiomer/s thereof.

As noted above, in certain embodiments, the inhibitor/s of the invention may be peptides, specifically, peptides derived from the N′ loop extension of PstS. An ‘isolated polypeptide’ is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms. By definition, isolated peptides are also non-naturally occurring, synthetic peptides. Methods for isolating or synthesizing peptides of interest with known amino acid sequences are well known in the art.

An ‘amino acid/s’ or an ‘amino acid residue/s’ can be a natural or non-natural amino acid residue/s linked by peptide bonds or bonds different from peptide bonds. The amino acid residues can be in D-configuration or L-configuration (referred to herein as D- or L-enantiomers). An amino acid residue comprises an amino terminal part (NH₂) and a carboxy terminal part (COOH) separated by a central part (R group) comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature are listed in 37 C.F.R. 1.822(b)(2). Examples of non-natural amino acids are also listed in 37 C.F.R. 1.822(b)(4), other non-natural amino acid residues include, but are not limited to, modified amino acid residues, L-amino acid residues, and stereoisomers of D-amino acid residues. Naturally occurring amino acids may be further modified, e.g. hydroxyproline, γ-carboxyglutamate, and O-phosphoserine.

Further, amino acids may be amino acid analogs or amino acid mimetics. Amino acid analogs refer to compounds that have the same fundamental chemical structure as naturally occurring amino acids, but modified R groups or modified peptide backbones, e.g. homoserine, norleucine, methionine sulfoxide, methionine methyl sulfonium. Amino acid mimetics refers to chemical compounds that have a structure that is different from the general chemical structure of an amino acid, but that function in a manner similar. Amino acids may be referred to herein by either their commonly known three letter symbols or by the one-letter symbols recommended by the IUPAC-IUB Biochemical Nomenclature Commission.

Further, peptides of the invention may comprise ‘equivalent amino acid residues’. This term refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, equivalent amino acid residues can be regarded as conservative amino acid substitutions.

In the context of the present invention, within the meaning of the term ‘equivalent amino acid substitution’ as applied herein, is meant that in certain embodiments one amino acid may be substituted for another within the groups of amino acids indicated herein below:

i) Amino acids having polar side chains (Asp, Glu, Lys, Arg, His, Asn, GIn, Ser, Thr, Tyr, and Cys);
ii) Amino acids having non-polar side chains (Gly, Ala, Val, Leu, lie, Phe, Trp, Pro, and Met);
iii) Amino acids having aliphatic side chains (Gly, Ala Val, Leu, ile);
iv) Amino acids having cyclic side chains (Phe, Tyr, Trp, His, Pro);
v) Amino acids having aromatic side chains (Phe, Tyr, Trp);
vi) Amino acids having acidic side chains (Asp, Glu);
vii) Amino acids having basic side chains (Lys, Arg, His);
viii) Amino acids having amide side chains (Asn, GIn);
ix) Amino acids having hydroxy side chains (Ser, Thr);
x) Amino acids having sulphur-containing side chains (Cys, Met);
xi) Neutral, weakly hydrophobic amino acids (Pro, Ala, Gly, Ser, Thr);
xii) Hydrophilic, acidic amino acids (GIn, Asn, Glu, Asp), and
xiii) Hydrophobic amino acids (Leu, lie, Val).

A Venn diagram is another method for grouping of amino acids according to their properties (Livingstone & Barton, CABIOS, 9, 745-756, 1993). In another preferred embodiment one or more amino acids may be substituted with another within the same Venn diagram group.

Still further, peptides of the invention may have secondary modifications, such as phosphorylation, acetylation, glycosylation, sulfhydryl bond formation, cleavage and the likes, as long as said modifications retain the functional properties of the original protein. Secondary modifications are often referred to in terms of relative position to certain amino acid residues. For example, a certain sequence positioned carboxyl-terminal to a reference sequence within a polypeptide is located proximal to the carboxyl terminus of the reference sequence, but is not necessarily at the carboxyl terminus of the complete polypeptide.

As shown in Example 7, ectopic expression of the PstS N-loop using an expression vector that comprise a nucleic acid sequence encoding the same, effectively reduced biofilm formation.

Thus, in some alternative embodiments, the inhibitor of the invention may be at least one isolated and purified nucleic acid sequence encoding the N′ loop extension of PstS or any fragment thereof or any vector comprising said nucleic acid sequence.

In more specific embodiments, the nucleic acid sequence encodes the N′ loop extension of P. aeruginosa PstS or any fragment thereof.

Still further embodiments relate to nucleic acid sequences that encode an N′ loop extension comprises residues 25 to 39 of P. aeruginosa PstS, as denoted by SEQ ID NO. 2 or any fragment thereof.

In certain embodiments, the inhibitor/s of the invention may comprise a nucleic acid sequence encoding the amino acid sequence as denoted by SEQ ID NO. 25 or any fragment/s thereof. In yet another embodiment, the inhibitor/s of the invention may comprise a nucleic acid sequence encoding the amino acid sequence as denoted by SEQ ID NO. 26. Still further, the inhibitor/s of the invention may comprise a nucleic acid sequence encoding the amino acid sequence as denoted by SEQ ID NO. 27. In further embodiments, the inhibitor/s of the invention may comprise a nucleic acid sequence encoding the amino acid sequence as denoted by SEQ ID NO. 28. Other embodiments relate to the inhibitor/s of the invention that may comprise a nucleic acid sequence encoding the amino acid sequence as denoted by SEQ ID NO. 29. In further embodiments, the inhibitor/s of the invention may comprise a nucleic acid sequence encoding the amino acid sequence as denoted by SEQ ID NO. 30. Still further, the invention provides inhibitor/s that may comprise a nucleic acid sequence encoding the amino acid sequence as denoted by SEQ ID NO. 23.

As indicated above, Example 7 shows that ectopic expression of the N terminal portion of PA PstS clearly inhibited biofilm formation. Thus, in certain specific embodiments, the invention further provide an inhibitor that comprises a nucleic acid sequence encoding the amino acid sequence of residues 1-38 of PA PstS:

SEQ ID NO. 50
MKLKRLMAALTFVAAGVGAASAVAAIDPALPEYQKASG,
as denoted by.

As used herein, the term ‘polynucleotide’ or a ‘nucleic acid sequence’ refers to a polymer of nucleic acids, such as deoxyribonucleic acid (DNA) or ribonucleic acid (RNA). As used herein, ‘nucleic acid’ (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase).

In this connection an ‘isolated polynucleotide’ is a nucleic acid molecule that is separated from the genome of an organism. For example, a DNA molecule that encodes the N′ loop of PstS or any fragment thereof that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

The invention further relates to recombinant DNA constructs comprising the polynucleotides of the invention or splice variants, homologues or derivatives thereof. The constructs of the invention may further comprise additional elements such as promoters, regulatory and control elements, translation, expression and other signals, operably linked to the nucleic acid sequence of the invention. As used herein, the term “recombinant DNA” or “recombinant gene” refers to a nucleic acid comprising an open reading frame encoding one of the proteins of the invention.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

Accordingly, the term control and regulatory elements includes promoters, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding any desired protein using the method of this invention.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

In some specific embodiments, the inhibitor/s of the invention may be a construct encoding the N′ loop extension of PA PstS, or of any fragments and derivatives thereof. Such construct may be constructed in any vector as described above. In certain and specific embodiments, the vector may be the pUCP18Ap. More particular embodiments include an inhibitor that may be the construct as described in Example 7.

In some other alternative embodiments, the inhibitor/s of the invention may be a compound that is directed against the N′ loop extension of PstS, specifically, a compound that specifically recognizes and binds the N′ loop extension of PstS. Thus, in some particular and non-limiting embodiments, the inhibitor/s of the invention may be at least one isolated and purified antibody that specifically recognizes and binds the N′ loop extension of PstS or any fragment thereof.

In more specific embodiments, such antibody specifically binds the N′ loop extension of P. aeruginosa PstS or any fragment thereof.

More specifically, the N′ loop extension comprises residues 25 to 39 of P. aeruginosa PstS, as denoted by SEQ ID NO. 2 or any fragment thereof.

It should be noted that the invention further encompass any antibody directed to any one of peptides 1-6 as denoted by SEQ ID NO. 25 to 30, as well as any derivative/s, enantiomers or fragment/s thereof that may include for example the sequence of SEQ ID NO. 23, 24, 50 and 56.

The term ‘antibody’ as meant herein encompasses the whole antibodies as well as any antigen binding fragment (i.e., ‘antigen-binding portion’) or single chain thereof. An ‘antibody’ refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region (abbreviated herein as CH). Each light chain is comprised of a light chain variable region (abbreviated herein as VL) and a light chain constant region (abbreviated herein as CL). The VH and VL regions can be further subdivided into regions of hypervariability, termed ‘complementarity determining regions’ (CDRs), interspersed with regions that are more conserved, termed “framework regions” (FRs). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system [e.g., effector cells) and the first component (C1q) of the classical complement system.

The term ‘antigen-binding portion’ of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term ‘antigen-binding portion’ of an antibody include (i) a Fab fragment, a monovalent fragment consisting of the VL, VH, CL and Cm domains; (ii) a F(ab′)2 fragment, a bivalent fragment comprising two Fab fragments linked by a disulfide bridge at the hinge region; (iii) a Fd fragment consisting of the VH and Cm domains; (iv) a Fv fragment consisting of the VL and VH domains of a single arm of an antibody, (v) a dAb fragment, which consists of a VH domain; (vi) an isolated complementarity determining region (CDR), and (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. Such single chain antibodies are also intended to be encompassed within the term “antigen-binding portion” of an antibody. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. These antibody fragments are obtained using conventional techniques known to those with skill in the art, and the fragments are screened for utility in the same manner as are intact antibodies.

An ‘antibody fragment’ is a portion of an antibody such as F(ab′)2, F(ab)2, Fab′, Fab, and the like. Regardless of structure, an antibody fragment binds with the same antigen that is recognized by the intact antibody. For example, an anti-(polypeptide according to the present invention) monoclonal antibody fragment binds an epitope of a polypeptide according to the present invention. The term ‘antibody fragment’ also includes a synthetic or a genetically engineered polypeptide that binds to a specific antigen, such as polypeptides consisting of the light chain variable region, ‘Fv’ fragments consisting of the variable regions of the heavy and light chains, recombinant single chain polypeptide molecules in which light and heavy variable regions are connected by a peptide linker (‘scFv proteins’), and minimal recognition units consisting of the amino acid residues that mimic the hypervariable region.

The term ‘epitope’ means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

Methods for preparing antibodies are known to the art. See, for example, Harlow & Lane (1988) Antibodies: a Laboratory Manual, Cold Spring Harbor Lab., Cold Spring Harbor, N.Y.). Monoclonal antibodies may be prepared from a single B cell line taken from the spleen or lymph nodes of immunized animals, in particular rats or mice, by fusion with immortalized B cells under conditions which favor the growth of hybrid cells. The technique of generating monoclonal antibodies is described in many articles and textbooks, such as the above-noted Chapter 2 of Current Protocols in Immunology. Spleen or lymph node cells of these animals may be used in the same way as spleen or lymph node cells of protein-immunized animals, for the generation of monoclonal antibodies as described in Chapter 2 therein. The techniques used in generating monoclonal antibodies are further described in by Kohler and Milstein, Nature 256; 495-497, (1975), and in U.S. Pat. No. 4,376,110. Antibodies that are isolated from organisms other than humans, such as mice, rats, rabbits, cows, can be made more human-like through chimerization or humanization.

In a further aspect, the invention provides an isolated and purified peptide comprising the amino acid sequence of the N′ loop extension of P. aeruginosa PstS and any derivative/s, enantiomer/s and fragment/s thereof.

In some embodiments, the N′ loop extension comprises residues 25 to 39 of P. aeruginosa PstS, as denoted by SEQ ID NO. 2 or any derivative/s, enantiomer/s or fragment/s thereof.

In some further embodiments, the peptide comprises the amino acid sequence Xaa(ₙ)-Ala-Ile-Asp-Pro-Ala-Leu-Pro-Glu-Xaa₍ₙ₎ as denoted by SEQ ID NO. 23 or any fragment/s, enantiomer/s or derivative/s thereof, wherein Xaa is any amino acid and n is zero or an integer of from 1 to 10.

In certain embodiments where n is zero, the peptide of the invention may comprise the amino acid sequence Ala-Ile-Asp-Pro-Ala-Leu-Pro-Glu as denoted by SEQ ID NO. 27 or any fragment/s, enantiomer/s or derivative/s thereof. In some further embodiments, at least one amino acid residue of an enantiomer of the peptide of SEQ ID NO. 27, may be a D-enantiomer.

In yet some further embodiments, the N-terminal Ala and the C terminal Glu of the peptide of the invention are D-enantiomers, said peptide comprises the amino acid sequence as denoted by SEQ ID NO. 56. In some embodiments, the enantiomer derivatives of the invention may exhibit enhanced stability and decreased sensitivity to proteolytic degradation.

In some other embodiments, the peptide comprises the amino acid sequence Ala-Ile-Asp-Pro-Ala-Leu-Pro-Glu-Xaa₍ₙ₎ as denoted by SEQ ID NO. 24 or any fragment or derivatives thereof, wherein Xaa is any amino acid and n is zero or an integer of from 1 to 10.

The peptide may comprise according to other embodiments, the amino acid sequence Ala-Ile-Asp-Pro-Ala-Leu-Pro-Glu-Tyr-Gln-Lys-Ala-Ser as denoted by SEQ ID NO. 26 or any fragment/s, enantiomer/s or derivative/s thereof.

In further embodiments, the peptide of the invention may comprise the amino acid sequence Ala-Ile-Asp-Pro-Ala-Leu-Pro-Glu-Tyr-Gln-Lys-Ala-Ser-Gly-Val-Ser-Gly as denoted by SEQ ID NO. 25 or any fragment or derivatives thereof.

Still further, the peptide of the invention may comprise the amino acid sequence of any one of Pro-Glu-Tyr-Gln-Lys as denoted by SEQ ID NO. 28, Glu-Tyr-Gln-Lys, as denoted by SEQ ID NO. 29 and Tyr-Gln-Lys-Ala-Ser-Gly-Val-Ser-Gly as denoted by SEQ ID NO. 30, or any fragment or derivatives thereof.

The invention further encompasses any derivatives, analogues, variants or homologues of any of the peptides disclosed herein. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention.

It should be noted that the polypeptides according to the invention can be produced either synthetically, or by recombinant DNA technology. Methods for producing polypeptides peptides are well known in the art.

In some embodiments, derivatives include, but are not limited to, polypeptides that differ in one or more amino acids in their overall sequence from the polypeptides defined herein, polypeptides that have deletions, substitutions, inversions or additions.

In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the invention may occur in any position of the modified peptide, as well as in any of the N′ or C′ termini thereof. It should be appreciated that in cases the deletion/s or insertion/s are in the N or C-terminus of the peptide, such derivatives may be also referred to as fragments. More specifically, in some embodiments the peptides of SEQ ID NO. 30, 29, 28, 27 and 26 may be considered as fragments of the peptide of SEQ ID NO. 25.

The peptides of the invention may all be positively charged, negatively charged or neutral. In addition, they may be in the form of a dimer, a multimer or in a constrained conformation, which can be attained by internal bridges, short-range cyclizations, extension or other chemical modifications.

The polypeptides of the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue.

Further, the peptides may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids. As an example for such extension, the peptide may be extended at the N-terminus and/or C-terminus thereof with an N-acetyl group.

For every single peptide sequence defined by the invention and disclosed herein, this invention includes the corresponding retro-inverse sequence wherein the direction of the peptide chain has been inverted and wherein all or part of the amino acids belong to the D-series.

In yet some further embodiments, the peptides of the invention may comprise at least one amino acid residue in the D-form. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations.

Only L-amino acids are manufactured in cells and incorporated into proteins. Some D-amino acids are found in the cell walls of bacteria, but not in bacterial proteins.

As noted above, Glycine, the simplest amino acid, has no enantiomers as it has two hydrogen atoms attached to the central carbon atom. Only when all four attachments are different can enantiomers occur.

It should be appreciated that in some embodiments, the enantiomer or any derivatives of the inhibitor peptides of the invention may exhibit enhanced activity, and superiority. In more specific embodiments, such derivatives and enantiomers may exhibit increased affinity, enhanced stability, and increased resistance to proteolytic degradation.

The invention also encompasses any homologues of the polypeptides specifically defined by their amino acid sequence according to the invention. The term “homologues” is used to define amino acid sequences (polypeptide) which maintain a minimal homology to the amino acid sequences defined by the invention, e.g. preferably have at least about 65%, more preferably at least about 70%, at least about 75%, even more preferably at least about 80%, at least about 85%, most preferably at least about 90%, at least about 95% overall sequence homology with the amino acid sequence of any of the polypeptide as structurally defined above, e.g. of a specified sequence, more specifically, an amino acid sequence of the polypeptides as denoted by any one of SEQ ID NO. 25, 26, 27, 28, 29, 30 and 56.

More specifically, “Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art and disclosed herein before. Such conservatively modified variants are in addition to and do not exclude polymorphic variants, interspecies homologues, and alleles and analogous peptides of the invention.

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved.

As noted above, the peptides of the invention may be modified by omitting their N-terminal sequence. It should be appreciated that the invention further encompasses the omission of about 1, 2, 3, 4, 5, 6, 7, 8 and more amino acid residues from both, the N′ and/or the C′ termini of the peptides of the invention.

In certain embodiments the peptide compounds of the invention may comprise one or more amino acid residue surrogate. An “amino acid residue surrogate” as herein defined is an amino acid residue or peptide employed to produce mimetics of critical function domains of peptides. Examples of amino acid surrogate include, but are not limited to chemical modifications and derivatives of amino acids, stereoisomers and modifications of naturally occurring amino acids, non-protein amino acids, post-translationally modified amino acids, enzymatically modified amino acids, and the like. Examples also include dimers or multimers of peptides. An amino acid surrogate may also include any modification made in a side chain moiety of an amino acid. This thus includes the side chain moiety present in naturally occurring amino acids, side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs or structures designed to mimic amino acids, and the like.

In some embodiments, derivatives of the peptides according to the invention may comprise an amino acid side chain moiety. A “derivative of an amino acid side chain moiety”, as used herein, is a modification to or variation in any amino acid side chain moiety, including a modification to or variation in either a naturally occurring or unnatural amino acid side chain moiety, wherein the modification or variation includes: (a) adding one or more saturated or unsaturated carbon atoms to an existing alkyl, aryl, or aralkyl chain; (b) substituting a carbon in the side chain with another atom, preferably oxygen or nitrogen; (c) adding a terminal group to a carbon atom of the side chain, including methyl (—CH₃), methoxy (—OCH₃), nitro (—NO₂), hydroxyl (—OH), or cyano (—C═N); (d) for side chain moieties including a hydroxy, thio or amino groups, adding a suitable hydroxy, thio or amino protecting group; or (e) for side chain moieties including a ring structure, adding one or ring substituents, including hydroxyl, halogen, alkyl, or aryl groups attached directly or through an ether linkage. For amino groups, suitable amino protecting groups include, but are not limited to, Z, Fmoc, Boc, Pbf, Pmc and the like.

The peptide according to the invention may comprise an “N-Substituted Amino Acid”. An “N-substituted amino acid”, as described herein, includes any amino acid wherein an amino acid side chain moiety is covalently bonded to the backbone amino group, optionally where there are no substituents other than H in the α-carbon position. Sarcosine is an example of an N-substituted amino acid. By way of example, sarcosine can be referred to as an N-substituted amino acid derivative of Ala, in that the amino acid side chain moiety of sarcosine and Ala is the same, methyl.

In the course of a reaction of peptide synthesis, a nitrogen protecting group may be used. As used herein, “a nitrogen protecting group” means a group that replaces an amino hydrogen for the purpose of protecting against side reactions and degradation during a reaction sequence, for example, during peptide synthesis. Solid phase peptide synthesis involves a series of reaction cycles comprising coupling the carboxy group of an N-protected amino acid or surrogate with the amino group of the peptide substrate, followed by chemically cleaving the nitrogen protecting group so that the next amino-protected synthon may be coupled. Nitrogen protecting groups useful in the invention include nitrogen protecting groups well known in solid phase peptide synthesis, including, but not limited to, t-Boc (tert-butyloxycarbonyl), Fmoc (9-flourenylmethyloxycarbonyl), 2-chlorobenzyloxycarbonyl, allyloxycarbonyl (alloc), benzyloxycarbonyl, 2-(4-biphenylyl)propyl-2-oxycarbonyl (Bpoc), 1-adamantyloxycarbonyl, trityl (triphenylmethyl), and toluene sulphonyl.

In one embodiment, one amino acid surrogate may be employed in a peptide of the invention, two amino acid surrogates may be employed in a peptide of the invention, or more than two amino acid surrogates may be employed in a peptide of the invention.

In another embodiment, there is provided a peptide including an amino acid surrogate wherein one or more peptide bonds between amino acid residues are substituted with a non-peptide bond.

In another embodiment of the invention, there is provided a peptide including at least one amino acid surrogate and a plurality of amino acid residues wherein the compound is a cyclic compound, cyclized by a bond between side chains of two amino acid residues, between an amino acid residue side chain and a group of an amino acid surrogate, between groups of two amino acid surrogate, between a terminal group of the compound and an amino acid residue side chain, or between a terminal group of the compound and a group of an amino acid surrogate.

In another embodiment, the peptide of the invention may include C-Terminus Capping Group. The term “C-terminus capping group” includes any terminal group attached through the terminal ring carbon atom or, if provided, terminal carboxyl group, of the C-terminus of a compound. The terminal ring carbon atom or, if provided, terminal carboxyl group, may form a part of a residue, or may form a part of an amino acid surrogate. In a preferred aspect, the C-terminus capping group forms a part of an amino acid surrogate which is at the C-terminus position of the compound. The C-terminus capping group includes, but is not limited to, —(CH₂)ₙ—OH, —(CH₂)ₙ—C(—O)—OH, —(CH₂)ₘ—OH, —(CH₂)ₙ—C(—O)—N(v₁)(v₂), —(CH₂)ₙ—C(—O)—(CH₂)ₘ—N(v₁)(v₂), —(CH₂)ₙ—O—(CH₂)ₘ—CH₃, —(CH₂)ₙ—C(—O)—NH—(CH₂)ₘ—CH₃, —(CH₂)ₙ—C(—O)—NH—(CH₂)ₘ—N(v₁)(v₂), —(CH₂)ₙ—C(—O)—N—((CH₂)ₘ—N(v₁)(v₂))₂, —(CH₂)ₙ—C(—O)—NH—CH(—C(—O)—OH)—(CH₂)ₘ—N(v₁)(v₂), —C(—O)—NH—(CH₂)ₘ—NH—C(—O)—CH(N(v₁)(v₂))((CH₂)ₘ—N(v₁)(v₂)), or —(CH₂)ₙ—C(—O)—NH—CH(—C(—O)—NH₂)—(CH₂)ₘ—N(v₁)(v₂), including all (R) or (S) configurations of the foregoing, where v₁ and v₂ are each independently H, a C₁ to C₁₇ linear or branched alkyl chain, m is 0 to 17 and n is 0 to 2; or any omega amino aliphatic, terminal aryl or aralkyl, including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, or any single natural or unnatural a-amino acid, beta-amino acid or a,a-disubstituted amino acid, including all (R) or (S) configurations of the foregoing, optionally in combination with any of the foregoing non-amino acid capping groups.

Still further embodiments relates to the peptides of the invention having an N-Terminus Capping Group. The term “N-terminus capping group” includes any terminal group attached through the terminal amine of the N-terminus of a compound. The terminal amine may form a part of a residue, or may form a part of an amino acid surrogate. In a preferred aspect, the N-terminus capping group forms a part of an amino acid surrogate which is at the N-terminus position of the compound. The N-terminus capping group includes, but is not limited to, any omega amino aliphatic, acyl group or terminal aryl or aralkyl including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 12-Ado, 7′-amino heptanoyl, 6-Ahx, Amc or 8-Aoc, or alternatively an N-terminus capping group is —(CH₂)ₘ—NH(v₃), —(CH₂)ₘ—CH₃, —C(—O)—(CH₂)ₘ—CH₃, —C(—O)—(CH₂)ₘ—NH(v₃), —C(—O)—(CH₂)ₘ—C(—O)—OH, —C(—O)—(CH₂)ₘ—C(—O)—(v₄), —(CH₂)ₘ—C(—O)—OH, —(CH₂)ₘ—C(—O)—(v₄), C(—O)—(CH₂)ₘ—O(v₃), —(CH₂)ₘ—O(v₃), C(—O)—(CH₂)ₘ—S(v₃), or —(CH₂)ₘ—S(v₃), where v₃ is H or a C₁ to C₁₇ linear or branched alkyl chain, and v₄ is a C₁ to C₁₇ linear or branched alkyl chain and m is 0 to 17.

It should be appreciated that the invention further encompass any of the peptides of the invention referred herein, any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

It should be noted that the present invention encompasses any fragment, derivative or analogue of any of the polypeptides of the invention. In certain embodiments, any of the polypeptides of the invention and derivatives thereof, possess the ability to inhibit biofilm formation.

As used herein, the term “functional fragment”, “functional mutant”, “functional derivative” or “functional variant” refers to an amino acid sequence which possesses biological function or activity that is identical to the activity possessed by the original polypeptides of the invention, specifically, the peptides comprising the amino acid sequence of any one of SEQ ID NO. 25, 26, 27, 28, 29, 30 and 56, may possess the activity of inhibiting biofilm formation. Such activity may be identified through a defined functional assay, as exemplified in the examples.

In a further aspect, the invention relates to an isolated and purified nucleic acid sequence encoding the N′ loop extension of P. aeruginosa PstS or any fragment thereof.

The invention further provides an expression vector comprising a purified nucleic acid sequence encoding the N′ loop extension of P. aeruginosa PstS or any fragment thereof.

It should be appreciated that any suitable vector may be applicable forth present invention. Non-limiting example for vectors are described herein before.

A further aspect of the invention relates to a composition comprising at least one inhibitor of a bacterial biofilm formation, wherein said inhibitor comprises at least one of:

(a) at least one amino acid sequence derived from the N′ loop extension of PstS or any ortholog, or of any fragment/s thereof, or any nucleic acid sequence encoding the same; and
(b) at least one compound that specifically binds to said N′ loop extension of PstS;
said composition optionally further comprises at least one pharmaceutically acceptable carriers, excipients, auxiliaries, and/or diluents.

In some embodiments, the N′ loop extension comprises residues 25 to 39 of P. aeruginosa PstS, as denoted by SEQ ID NO. 2 or any derivative/s, enantiomer/s or fragment/s thereof.

In some specific embodiments, the composition of the invention may comprise at least one of:

(a) at least one isolated and purified peptide comprising the amino acid sequence of any one of SEQ ID NO. 25-30, 23, 24 or 50, or of any fragment or derivatives thereof;
(b) at least one isolated and purified nucleic acid sequence encoding the N′ loop extension of P. aeruginosa PstS or any fragment thereof, or any expression vector comprising said nucleic acid sequence;
(c) at least one isolated and purified antibody that specifically recognizes and binds the N′ loop extension of PstS or any fragment thereof; and
(d) any combinations of (a), (b) and (c).

It should be noted that in some specific embodiments, the composition of the invention may comprise any of the inhibitors described above in an amount effective for inhibiting bacterial biofilm formation.

The invention further provides in some embodiments, the composition as described above for use in a method for inhibiting, reducing or eliminating bacterial biofilm formation.

In further specific embodiments, the composition of the invention may be a pharmaceutical composition for treating, preventing, ameliorating, reducing or delaying the onset of an infectious condition in a subject in need thereof.

In some specific embodiments, the composition of the invention, as well as the methods described herein after, may be specifically applicable for treating infectious conditions caused by biofilm forming bacteria.

Many bacteria can grow and live as biofilms, including Gram-negative as well as Gram-positive bacteria. Biofilms may form on living or non-living surfaces and can be prevalent in natural, industrial and hospital settings. Notable examples include, although not limited to:

Gonococcal biofilms (e.g. Neisseria gonorrhoeae spp., a Gram-negative human pathogen) can form biofilms on glass surfaces and over human cells. There is evidence for formation of gonococcal biofilms on human cervical epithelial cells during natural disease.
Dental plaque which is a complex biofilm containing several hundred different species of bacteria. While these are normally harmless commensals, shifts in the population structure can lead to the plaque-related diseases such as dental caries and periodontal disease. Surface polysaccharides are important in coaggregation and aid co-colonisation of particular species.
Oral microbial communities, most of the bacterial species found in the mouth are capable of forming microbial biofilms, a feature of which is inter-bacterial communication through direct cell-cell contact mediated by specific protein’ ‘adhesions’, and in the case of inter-species aggregation—by complementary polysaccharide receptors.
Gram-positive biofilm infections, wherein biofilm is the default mode of growth for most if not all bacterial species, which has profound consequences in numerous clinical settings. Several mechanisms involving surface proteins and carbohydrate-containing structures have been implicated in biofilm formation of Gram-positive bacteria. Recent evidence indicates that extracellular DNA may also be involved in this process.
Biofilms on intravascular devices, including central venous catheters (CVCs) have been well documented. Both Gram-positive and Gram-negative bacteria have been isolated from biofilms on CVCs. Colonization of the outer lumen of the catheter is usually the result of the catheter's proximity to skin flora. Colonization of the inner lumen of catheters (specifically by Gram-negative rods) may be the result of a break in aseptic handling of the device prior to insertion or of the exposure of the end connectors to water, soil, or contaminated intravenous (i.v.) fluids.
Biofilm by Vibrio cholerae spa., the causative agent of cholera forms biofilms on diverse surfaces. This ability to form biofilms appears to be critical for the environmental survival and the transmission of V. cholerae.
Biofilms in Pasteurellaceae, a family of Gram-negative, facultatively anaerobic, rod-shaped bacteria that are mostly commensals of mucosal surfaces but are capable of causing opportunistic infections and disease. Biofilms are produced by many members of this group, and these surface-attached biofilm communities may promote bacterial persistence in vivo, even in the face of immune effectors and antimicrobial treatment.

The microbial cells growing in a biofilm are physiologically distinct from planktonic cells of the same organism, which, by contrast, are single-cells that may float or swim in a liquid medium. In this connection, the term (or ‘bacterial swarming motility’) is often used to denote a mechanism oppositely regulated and antagonistic to biofilm formation. Swarming is a common yet specialized form of surface translocation exhibited by flagellated bacteria, such as Pseudomonas aeruginosa (PA, monoflagellated bacteria, Escherichia coli (E. coli, a peritrichous bacteria, i.e. having a uniform distribution of flagella), and Salmonella enterica. Apart from flagella, swarming further requires an increase in flagellar biosynthesis, cell-cell interactions, and also the presence of a surfactant.

P. aureginosa (PA) are common Gram-negative bacteria that can cause disease in animals and humans. It is found in soil, water, skin flora, and most man-made environments throughout the world. It thrives not only in normal atmospheres, but also in hypoxic atmospheres, and has, thus, colonized many natural and artificial environments. PA has become an important cause of infection, especially in patients with compromised host defense mechanisms. It is the most common pathogen isolated from patients who have been hospitalized longer than 1 week, and it is a frequent cause of nosocomial infections. Pseudomonal infections are complicated and can be life-threatening. In certain embodiments, the inhibition of the invention as well as the compositions of the invention may be applicable in inhibiting biofilm formation. By inhibiting biofilm formation, the compositions and methods of the invention may be applicable for treating any pathogenic condition caused by a biofilm forming bacteria, specifically, PA.

More specifically, the common clinical conditions caused by PA infections may include:

Eye infections, most commonly involving the cornea (keratiitis) but may occasionally involve the intraocular cavity (endophthalmitis). Bacteria are introduced into the eye by trauma or following corneal injury caused for example by contact lenses.
Ear infections, ‘Swimmer's ear’ is an infection of the outer ear canal that develops when water remains in the ear after swimming. Malignant otitis exteerna is a severe infection that occurs when bacteria in the ear canal invade through the surrounding cartilage to deeper structures, including middle ear, mastoid air cells and temporal bone.
Chronic respiratory infections, PA is commonly isolated from the respiratory tracts of individuals with cystic fibrosis and is associated with an accelerated decline in lung function in these patients. Chronic lung colonization and infection also occur in bronchiectasis, a disease of the bronchial tree, and in chronic obstructive pulmonary disease, a disease characterized by narrowing of the airways and abnormalities in air flow.
Hospital-acquired pneumonia, PA is one of the most common causes of pneumonia in hospitalized patients, especially in mechanically ventilated patients. It is associated with a particularly high mortality rate.
Complicated abdominal infections, PA is identified in some cases of hostpital-acquired complicated intra-abdominal infections.
Urinary tract infections, PA accounts for a substantial portion of nosocomial urinary tract infections. These infections are usually associated with a foreign body or surgery of the urinary tract.
Blood stream infections, PA causes a substantial proportion of nosocomial blood stream infections, which can be associated with ecthyma gangrenosum, a painless nodular skin lesion with central ulceration and haemorrhage.
Skin and soft tissue infections, PA can survive in hot tubs and infect macerated skin, leading to ‘hot tubs folliculitis’. PA also infects wounds of patients with burns and is a common cause of nosocomial skin and soft tissue infections.

Predisposing conditions to PA infections may include, although not limited to disrupted epithelial barrier (as found in a patient with a burn wound), depletion of neutrophils (for example, in a cancer patient receiving chemotherapy), presence of a foreign body (a patient with a central venous catheter), altered mucociliary clearance (in an individual with cystic fibrosis). It should be further noted that many PA infections occur after patients have been hospitalized.

Thus in specific embodiments, the compounds, compositions and methods of the present invention, described herein after, are particularly applicable to these conditions, by themselves or as a part of a larger therapeutic regimen.

Still further, the compositions and methods of the invention may be applicable to infectious conditions caused by other biofilm forming bacteria. In more specific embodiments, the compositions of the invention may be applicable for bacteria having an N′ loop extension of the PstS protein.

Thus, in certain embodiments, the compositions and method of the invention may be applicable for infections caused by C. perfringens.

C. perfringens (Clostridium perfringens, formerly known as C. welchii, or Bacillus welchii) are spore-forming Gram-positive bacteria. C. perfringens is found in many environmental sources as well as in the intestines of humans and animals; it commonly grows on raw meat and poultry. Some strains of C. perfringens produce toxin in the intestine. C. perfringens is one of the most common causes of food poisoning, estimated at nearly a million cases each year only in the US. Persons infected with C. perfringens develop diarrhea and abdominal cramps. The illness is not passed from one person to another. Everyone is susceptible to food poisoning from C. perfringens. The children and elderly are most at risk to develop more severe symptoms and complications including dehydration in severe cases.

In other embodiments, the compositions and method of the invention may be applicable for infections caused by S. phenumonia. S. phenumonia (Streptococcus pneumoniae, or pneumococcus), are Gram-positive bacteria and is a normal inhabitant of the human upper respiratory tract. S. pneumoniae can cause pneumonia, usually of the lobar type, paranasal sinusitis and otitis media, or meningitis, which is usually secondary to one of the former infections. It also causes osteomyelitis, septic arthritis, endocarditis, peritonitis, cellulitis and brain abscesses. S. pneumoniae is currently the leading cause of invasive bacterial disease in children and the elderly. It is known in medical microbiology as the pneumococcus, referring to its morphology and its consistent involvement in pneumococcal pneumonia. S. pneumoniae is also the most common cause of community-acquired pneumonia (CAP).

Still further embodiments relate to infections caused by M. tuberculosis. M. tuberculosis (Mycobacterium tuberculosis, MTB) is a pathogenic bacteria species in the family Mycobacteriaceae and the causative agent of most cases of tuberculosis. More specifically, The M. tuberculosis complex (MTC) consists of M. africanum, M. bovis, M. canettii, M. microti and M. tuberculosis. All species of mycobacteria have ropelike structures of peptidoglycan that are arranged in such a way to give them properties of acid fast bacteria. Mycobacteria are abundant in soil and water, but MTB specifically is mainly identified as a pathogen that lives in the host. Some species in MTC have adapted their genetic structure specifically to infect human populations. Since as many as 32% of the human population is affected by tuberculosis (TB), an airborne disease caused by infection of MTB in one way or another, and about 10% of them becomes ill per year.

Further embodiments of the invention relate to inhibitors applicable in Y. pestis infections. Y. pestis (Yersinia pestis, formerly Pasteurella pestis, plague) is a Gram-negative, rod-shaped coccobacillus, a facultative anaerobic bacterium that can infect humans and animals. Plague is a disease that affects humans and other mammals. Humans usually get plague after being bitten by a rodent flea that is carrying the plague bacterium or by handling an animal infected with plague. Plague is infamous for killing millions of people in Europe during the Middle Ages. Today, modern antibiotics are effective in treating plague. Without prompt treatment, the disease can cause serious illness or death. Presently, human plague infections continue to occur in the western United States, but significantly more cases occur in parts of Africa and Asia.

Still further, of particular interest for the compositions and methods of the invention are any bacteria involved in nosocomial infections. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality, and pose a significant economic burden. In view of the rising levels of antibiotic resistance and the increasing severity of illness of hospital in-patients, this problem needs an urgent solution. The nosocomial-infection pathogens could be subdivided into Gram-positive bacteria (Staphylococcus aureus, Coagulase-negative staphylococci), Gram-positive cocci (Enterococcus faecalis and Enterococcus faecium), Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). As noted above, among many others Pseudomonas aeruginosa is an extremely important nosocomial Gram-negative aerobic rod pathogen. The compositions and methods of the invention are particularly effective in treating Pseudomonas aeruginosa infections. As indicated above, the inhibitors of the invention may be applicable for any bacteria involving biofilm formation. Non-limiting examples of bacteria that involve in biofilm formation include members of the genus Actinobacillus (such as Actinobacillus actinomycetemcomitans), members of the genus Acinetobacter (such as Acinetobacter baumannii), members of the genus Aeromonas, members of the genus Bordetella (such as Bordetella pertussis, Bordetella bronchiseptica, or Bordetella parapertussis), members of the genus Brevibacillus, members of the genus Brucella, members of the genus Bacteroides (such as Bacteroides fragilis), members of the genus Burkholderia (such as Burkholderia cepacia or Burkholderia pseudomallei), members of the genus Borelia (such as Borelia burgdorferi), members of the genus Bacillus (such as Bacillus anthracis or Bacillus subtilis), members of the genus Campylobacter (such as Campylobacter jejuni), members of the genus Capnocytophaga, members of the genus Cardiobacterium (such as Cardiobacterium hominis), members of the genus Citrobacter, members of the genus Clostridium (such as Clostridium tetani or Clostridium difficile), members of the genus Chlamydia (such as Chlamydia trachomatis, Chlamydia pneumoniae, or Chlamydia psiffaci), a member of the genus Eikenella (such as Eikenella corrodens), members of the genus Enterobacter, members of the genus Escherichia (such as Escherichia coli), members of the genus Francisella (such as Francisella tularensis), members of the genus Fusobacterium, members of the genus Flavobacterium, members of the genus Haemophilus (such as Haemophilus ducreyi or Haemophilus influenzae), members of the genus Helicobacter (such as Helicobacter pylori), members of the genus Kingella (such as Kingella kingae), members of the genus Klebsiella (such as Klebsiella pneumoniae), members of the genus Legionella (such as Legionella pneumophila), members of the genus Listeria (such as Listeria monocytogenes), members of the genus Leptospirae, members of the genus Moraxella (such as Moraxella catarrhalis), members of the genus Morganella, members of the genus Mycoplasma (such as Mycoplasma hominis or Mycoplasma pneumoniae), members of the genus Mycobacterium (such as Mycobacterium tuberculosis or Mycobacterium leprae), members of the genus Neisseria (such as Neisseria gonorrhoeae or Neisseria meningitidis), members of the genus Pasteurella (such as Pasteurella multocida), members of the genus Proteus (such as Proteus vulgaris or Proteus mirablis), members of the genus Prevotella, members of the genus Plesiomonas (such as Plesiomonas shigelloides), members of the genus Pseudomonas (such as Pseudomonas aeruginosa), members of the genus Providencia, members of the genus Rickettsia (such as Rickettsia rickettsii or Rickettsia typhi), members of the genus Stenotrophomonas (such as Stenotrophomonas maltophila), members of the genus Staphylococcus (such as Staphylococcus aureus or Staphylococcus epidermidis), members of the genus Streptococcus (such as Streptococcus viridans, Streptococcus pyogenes (group A), Streptococcus agalactiae (group B), Streptococcus bovis, or Streptococcus pneumoniae), members of the genus Streptomyces (such as Streptomyces hygroscopicus), members of the genus Salmonella (such as Salmonella enteriditis, Salmonella typhi, or Salmonella typhimurium), members of the genus Serratia (such as Serratia marcescens), members of the genus Shigella, members of the genus Spirillum (such as Spirillum minus), members of the genus Treponema (such as Treponema pallidum), members of the genus Veillonella, members of the genus Vibrio (such as Vibrio cholerae, Vibrio parahaemolyticus, or Vibrio vulnificus), members of the genus Yersinia (such as Yersinia enter ocolitica, Yersinia pestis, or Yersinia pseudotuberculosis), and members of the genus Xanthomonas (such as Xanthomonas maltophilia).

The term “pharmaceutical composition” in the context of the invention means that the composition is of a grade and purity suitable for therapeutic administration to human subjects and is present together with at least one of carrier/s, diluent/s, excipient/s and/or additive/s that are pharmaceutically acceptable. The pharmaceutical composition may be suitable for any mode of administration whether oral or parenteral, by injection or by topical administration by inhalation, intranasal spray or intraocular drops.

Thus, some embodiments consider the composition/s according to the invention, particularly for treating respiratory diseases, specifically, chronic respiratory infections, for example in case of cystic fibrosis or pneumonia. According to one embodiment, such combined composition may be particularly adapted for pulmonary delivery. In more specific embodiments, such pulmonary delivery may be affected using nasal or oral administration, or any combination thereof. More specifically, pulmonary delivery may require the use of liquid nebulizers, aerosol-based metered dose inhalers (MDI's), or dry powder dispersion devices. Still further, it should not be overlooked that the composition of the invention, particularly when used for treating infections of burns, may be an acceptable topically applied composition as will be described in more detail herein after. Alternatively, the administration may be systemic such as by sublingual, rectal, vaginal, buccal, parenteral, intravenous, intramuscular, subcutaneous modes transdermal, inrtaperitoneal or intranasal modes of administration. However, oral, transmucosal, intestinal or parenteral delivery, including intramuscular, subcutaneous and intramedullary injections as well as rectal, intrathecal, direct intraventricular, intravenous, intraocular injections or any other medically acceptable methods of administration can be considered as well.

The compositions of the invention may comprise carriers suitable for pulmonary delivery that may involve nasal and/or oral administration. In specific embodiments, such carrier may be any one of spray, mist, patch, foam, alcoholic foam, oily foam, aqueous foam, bandage, membrane, gel, cream, emulsion, oily solution, aqueous solution, hydroethanolic solution, hydro-alcoholic-glycolic solution, mixture of alcohol and glycols, microemulsion, double emulsions, nanoemulsion, nanoparticles, microparticles, microcapsules, lipid particles, lipospheres, liposomes, lipid vesicles, solid lipid nanoparticles, liquid crystals, eutectic mixtures, eutectic crystsls, cubosomes, hexazomes, micelosomes, liposomal systems, vesicular systems, nanocubes, ethosomes, hydroethanolic systems, mixtures of alcohols and glycols, aqueous mixtures of alcohols and glycols, buffer solutions, polymer based delivery systems, hydrophilic or lipophilic suppository bases, chitosan and derivatives bases.

For nasal administration, suitable carriers are preferably water-soluble and include water, propylene glycol and other pharmaceutically acceptable alcohols, xanthan gum, locust bean gum, galactose, other saccharides, oligosaccharides and/or polysaccharides, starch, starch fragments, dextrins, British gum and mixtures thereof.

For buccal administration, suitable carriers are water-soluble carrier materials, for example (poly) saccharides like hydrolysed dextran, dextrin, mannitol, and alginates, or mixtures thereof, or mixtures thereof with other carrier materials like polyvinylalcohol, polyvinylpyrrolidine and water-soluble cellulose derivatives, like hydroxypropyl cellulose. In specific embodiments, the buccal carrier material may be gelatin, especially partially hydrolysed gelatin.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient. Suitable formulations wherein a semisolid carriers such as gel, cream or ointment may be also used for nasal administration according to the invention.

Specific embodiments of the invention contemplate skin infectious conditions, specifically, in case of burns. Therefore, treatment by topical administration of the affected skin areas of an ointment, cream, suspensions, paste, lotions, powders, solutions, oils, encapsulated gel, liposomes containing the inhibitor/s of the invention, any nano-particles containing the inhibitor/s of the invention, or sprayable aerosol or vapors containing a combination of these inhibitors, are also encompassed by the invention. Conventional pharmaceutical carriers, aqueous, powder or oily bases, thickeners and the like may be necessary or desirable. The term “topically applied” or “topically administered” means that the ointment, cream, emollient, balm, lotion, solution, salve, unguent, or any other pharmaceutical form is applied to some or all of that portion of the skin of the patient skin that is, or has been, affected by, or shows, or has shown, one or more symptoms of bacterial infectious disease, or any other symptoms involving the skin.

It should be noted that in certain embodiments, a topical application of the biofilm formation inhibitor/s by the method of the invention particularly in treating infected skin (for example, in case of burns), any transdermal delivery may be used. As used herein, the term “transdermal” refers to delivery, administration or application of a drug by means of direct contact with tissue, such as skin or mucosa. Such delivery, administration or application is also known as percutaneous, dermal, transmucosal and buccal.

Therapeutic compositions for transdermal administration, or “dermal compositions” are compositions which contain one or more drugs solubilized therein, specifically, any of the biofilm formation inhibitor/s, specifically, the N′ loop derived peptides or combinations thereof according to the invention. The composition is applied to a dermal area, for dermal administration or topical application of the drugs. Such a dermal composition may comprise a polymer matrix with the one or more drugs contained therein. The polymer matrix may be a pressure-sensitive adhesive for direct attachment to a user's (e.g., a patient's) skin. Alternatively, the polymer matrix may be non-adhesive and may be provided with separate adhesion means (such as a separate adhesive layer) for adhering the composition to the user's skin.

As used herein, “matrix” is defined as a polymer composition which incorporates a therapeutically effective amount of the drug therein. The matrix may be monolithic and comprise a pressure-sensitive adhesive, or it may use separate attachment means for adhering or holding to the user's skin, such as a separate adhesive layer. A dermal drug delivery system comprising a matrix may optionally include additional drug supply means for continuously replenishing the drug supply in the matrix. As used herein, a polymer is an “adhesive” if it has the properties of an adhesive per se, or if it functions as an adhesive by the addition of tackifiers, plasticizers, cross-linking agents or other additives.

Thus, the invention also contemplates the use according to the invention, wherein the at least one pharmaceutically acceptable carrier is adapted for transdermal administration, and the carrier may further comprise at least one agent for enhancing penetration through the skin. The term “skin” as used herein refers to the outer covering of a mammal body, comprising the epidermis and the dermis. More specifically, “skin” as used herein means the air-contacting part of the human body, to a depth of about 7 mm from the air interface; as such, it also includes the nails.

According to certain embodiments, an agent for enhancing penetration through the skin used by the invention may be used. Such agent may include any one of terpens, unsaturated acids, oleic acid, azone derivatives, surfactants, cetomacrogol, short chain alcohols, glycols, sulphoxides, alkyl sulphoxides, urea, sunscreen molecules, sunscreens in ethanolic solutions, short chain alcohols, glycols, or any combination thereof. The application of the transdermal patch and the flow of the active drug constituent from the patch to the circulatory system via skin occur through various methods described herein may involve active or passive delivery.

Still further, the compositions of the invention may also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives.

As noted above, it is understood that the compositions of the invention involves administration by any one of nasal, transdermal, pulmonary, oral, buccal or sublingual administration, or any combinations thereof. However, it should be appreciated that the compositions used by the invention, may be administered by injection (subcutaneously, intraperitoneally, intramuscularly, intravenously), rectally, vaginally, intraocular, sprayed at armpit and any combination thereof.

In many instances, therapies employing two or more administration methods are required to adequately address different medical conditions and/or effects of a certain disorder under treatment. Thus, at least one biofilm formation inhibitor/s of the invention or specifically, the peptides of the invention or any salts, esters or base thereof or any mixture thereof, may be administered by the method of the invention using a combination of at least two administration methods. Combining these at least two administration methods safely and effectively improves overall beneficial effect on the disorders addressed by this invention.

Thus, it is understood that according to some embodiments of the invention, the compositions of the invention may be adapted for oral administration, before, simultaneously with, after or any combination thereof, the intranasal or pulmonary administration of the compositions.

As indicated above, in addition to the intraperitoneal, intranasal and transdermal routes, the compositions used in the uses, methods of the invention may be adapted for administration by any other appropriate route, for example by the parenteral, oral (including buccal or sublingual), rectal, topical (including buccal or sublingual) or vaginal route. Such formulations may be prepared by any method known in the art of pharmacy, for example by bringing into association the active ingredient with the carrier(s) or excipient(s).

Pharmaceutical formulations adapted for rectal administration may be presented as suppositories or enemas.

Pharmaceutical formulations adapted for vaginal administration may be presented as pessaries, tampons, creams, gels, pastes, foams or spray formulations.

Compositions and formulations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis.

Formulations for ocular and aural administration may be formulated to be immediate and/or modified release. Modified release includes delayed, sustained, pulsed, controlled, targeted, and programmed release.

Preferred unit dosage formulations are those containing a daily dose or sub-dose, as herein above recited, or an appropriate fraction thereof, of an active ingredient.

In optional embodiments, the composition of the invention may further comprise a pharmaceutically acceptable carrier, excipient or diluent.

As noted above, any of the compositions of the invention may comprise pharmaceutically acceptable carriers, vehicles, adjuvants, excipients, or diluents. As used herein pharmaceutically acceptable carriers, vehicles, adjuvants, excipients, or diluents, are well-known to those skilled in the art and are readily available to the public. It is preferred that the pharmaceutically acceptable carrier be one which is chemically inert to the active compounds and one which has no detrimental side effects or toxicity under the conditions of use.

The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Each carrier should be both pharmaceutically and physiologically acceptable in the sense of being compatible with the other ingredients and not injurious to the subject. Formulations include those suitable for immersion, oral, parenteral (including subcutaneous, intramuscular, intravenous, intraperitoneal, implantation for slow release and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

A further aspect of the invention relates to a method for inhibiting, reducing or eliminating bacterial biofilm formation. More specifically, the invention provides methods for inhibiting and reducing biofilm formation in a subject, or alternatively in any surface, solid or semi-solid support or any material or substance. In some specific embodiments, the method comprising administering to said subject, or contacting, applying or dispensing to said surface, substance or material an effective amount of at least one inhibitor of a bacterial biofilm formation or any composition comprising the same. More specifically, in certain embodiments, the inhibitor comprises at least one of:

(a) at least one amino acid sequence derived from the N′ loop extension of PstS or any ortholog, or of any fragment thereof or any nucleic acid sequence encoding the same; and
(b) at least one compound that specifically binds to said N′ loop extension of PstS.

In some specific embodiments, the method of the invention may use any of the inhibitors defined by the invention.

As noted above, the invention provides compositions and methods (as well as kits that will be described herein after) for inhibiting biofilm formation in a subject, by administering the inhibitors of the invention to said subject in need, and thereby provides therapeutic application of the inhibitors described herein. In yet some additional embodiments, the invention provides methods for inhibiting biofilm formation on a surface, solid support or any other material or substance, and thereby further provides a prophylactic application for the inhibitors of the invention. These further applications of the inhibitors of the invention will be discussed and described in more detail herein after.

Still further aspect of the invention relates to a method for treating, preventing, ameliorating, reducing or delaying the onset of an infectious clinical condition in a subject in need thereof. More specifically, the method of the invention comprising the step of administrating to said subject a therapeutically effective amount of at least one inhibitor of a bacterial biofilm formation or of any composition comprising the same, wherein said inhibitor comprises at least one of:

(a) at least one amino acid sequence derived from the N′ loop extension of PstS, any ortholog, or of any fragment thereof or any nucleic acid sequence encoding the same; and
(b) at least one compound that specifically binds to said N′ loop extension of PstS.

In more specific embodiments, the method of the invention may use any of the inhibitors defined herein.

More specifically, in some specific embodiments, the inhibitors used by the methods of the invention may be peptides derived from the N-loop of PstS. More specifically, the N′ loop extension comprises residues 25 to 39 of P. aeruginosa PstS, as denoted by SEQ ID NO. 2 or any derivative or fragment thereof. Still further, in some embodiments, the inhibitor/s used by the methods of the invention may be at least one peptide derived from the N′ loop extension of the PA PstS. Such peptide may comprise according to certain embodiments of the invention, at least part of the amino acid sequence of the N′ loop extension of the PA PstS protein. It should be appreciated that the peptide may be extended either in the N′ or C′ termini thereof, or both, as described for example in the peptide comprising the amino acid sequence of Xaa₍ₙ₎₋Ala-Ile-Asp-Pro-Ala-Leu-Pro-Glu-Xaa₍ₙ₎ as denoted by SEQ ID NO. 23 or any fragment or derivative thereof, wherein Xaa is any amino acid and n is zero or an integer of from 1 to 10.

In some specific embodiments, the inhibitor used by the methods of the invention may be at least one isolated and purified peptide comprising the amino acid sequence Ala-Ile-Asp-Pro-Ala-Leu-Pro-Glu as denoted by SEQ ID NO. 27 or any fragment/s, enantiomers, or derivative/s thereof. It should be noted that in certain embodiments, this peptide is also referred to herein as Peptide-3.

It should be appreciated that derivatives of any of the peptides of the invention include also any enantiomers thereof. More specifically, such enantiomers may comprise at least one amino acid residue in D-form. In yet some further embodiments, the peptides of the invention may comprise at least two, at least three, at least four, at least five, at least six, at least seven, at least eight, at least nine, at least ten or more amino-acid residues in the D-form.

In some specific embodiments, at least one amino acid residue of an enantiomer of a peptide comprising the amino acid sequence as denoted by SEQ ID NO. 27, may be a D-enantiomer.

Of particular relevance for the methods of the invention is an enantiomer peptide of SEQ ID NO. 27, where the N-terminal Ala and the C terminal Glu of the peptide are D-enantiomers. In more specific embodiments the peptide comprises the amino acid sequence as denoted by SEQ ID NO.

56.

In some further embodiments, the inhibitor used by the methods of the invention may be at least one isolated and purified peptide comprising the amino acid sequence Ala-Ile-Asp-Pro-Ala-Leu-Pro-Glu-Xaa₍ₙ₎ as denoted by SEQ ID NO. 24 or any fragment/s, enantiomer/s or derivative/s thereof, wherein Xaa is any amino acid and n is zero or an integer of from 1 to 10.

In yet further specific embodiments, the inhibitor used by the methods of the invention may be at least one isolated and purified peptide comprising the amino acid sequence Ala-Ile-Asp-Pro-Ala-Leu-Pro-Glu-Tyr-Gln-Lys-Ala-Ser as denoted by SEQ ID NO. 26 or any fragment or derivatives thereof. It should be noted that in certain embodiments, this peptide is also referred to herein as Peptide-2.

Some further embodiments relate to the inhibitor used by the methods of the invention that may be at least one isolated and purified peptide comprising the amino acid sequence Ala-Ile-Asp-Pro-Ala-Leu-Pro-Glu-Tyr-Gln-Lys-Ala-Ser-Gly-Val-Ser-Gly as denoted by SEQ ID NO. 25 or any fragment or derivatives thereof. It should be noted that in certain embodiments, this peptide is also referred to herein as Peptide-1.

Still further, the inhibitor used by the methods of the invention may be at least one isolated and purified peptide comprising the amino acid sequence Pro-Glu-Tyr-Gln-Lys as denoted by SEQ ID NO. 28 or any fragment or derivatives thereof. It should be noted that in certain embodiments, this peptide is also referred to herein as Peptide-4.

In further embodiments the inhibitor used by the methods of the invention may be at least one isolated and purified peptide comprising the amino acid sequence Glu-Tyr-Gln-Lys, as denoted by SEQ ID NO. 29 or any fragment or derivatives thereof. It should be noted that in certain embodiments, this peptide is also referred to herein as Peptide-5.

Still further, the inhibitor used by the methods of the invention may be at least one isolated and purified peptide comprising the amino acid sequence Tyr-Gln-Lys-Ala-Ser-Gly-Val-Ser-Gly as denoted by SEQ ID NO. 30 or any fragment or derivatives thereof. It should be noted that in certain embodiments, this peptide is also referred to herein as Peptide-6.

In some embodiments, the subject in need is a subject suffering from a chronic or acute immune-related disorder. It should be noted that an “Immune-related disorder” is a condition that is associated with the immune system of a subject, either through activation or inhibition of the immune system, or that can be treated, prevented or reduced by targeting a certain component of the immune response in a subject, such as the adaptive or innate immune response. In more specific embodiments, the immune-related disorder may be any one of an infectious condition, autoimmune disease and a proliferative disorder, or any disorders or conditions associated therewith.

In yet some more specific embodiments, the methods of the invention are applicable for treating and preventing infectious conditions caused by any biofilm producing bacteria. More specifically, the methods of the invention may be particularly applicable for treating different clinical conditions caused by PA infections. Such conditions may include but are not limited to eye infections, ear infections, chronic respiratory infections, hospital-acquired pneumonia, complicated abdominal infections, urinary tract infections, blood stream infections and skin and soft tissue infections. The invention therefore provides compositions and method for treating and preventing these PA associated disorders.

It should be however appreciated that the methods of the invention provide appropriate treatment for any pathologic condition caused by any biofilm producing bacteria. In yet some more specific embodiments, the methods of the invention may be useful in treating and preventing conditions caused by bacteria expressing a PstS protein ortholog having an N-loop extension as specified by the invention. Such bacteria may include but are not limited to, C. perfringens, S. pneumonia, L. brevis, M. tuberculosis and Y. pestis.

The term “treatment” in accordance with disorders associated with infectious conditions may refer to one or more of the following: elimination, reducing or decreasing the intensity or frequency of disorders associated with said infectious condition. The treatment may be undertaken when disorders associated with said infection, incidence is beginning or may be a continuous administration, for example by administration every 1 to 14 days, to prevent or decrease occurrence of infectious condition in an individual prone to said condition. Such individual may be for example a subject having a compromised immune-system, in case of cancer patients undergoing chemotherapy or HIV infected subjects. Thus, the term “treatment” is also meant as prophylactic or ameliorating treatment.

The term “prophylaxis” refers to prevention or reduction the risk of occurrence of the biological or medical event, specifically, the occurrence or re occurrence of disorders associated with infectious disease, that is sought to be prevented in a tissue, a system, animal or human by a researcher, veterinarian, medical doctor or other clinician, and the term “prophylactically effective amount” is intended to mean that amount of a pharmaceutical composition that will achieve this goal. Thus, in particular embodiments, the methods of the invention are particularly effective in the prophylaxis, i.e., prevention of conditions associated with infectious disease. Thus, subjects administered with said compositions are less likely to experience symptoms associated with said infectious condition that are also less likely to re-occur in a subject who has already experienced them in the past.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with any one of an immune-related disorder and an infectious disease, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of an immune-related disorder or an infectious disease and their symptoms slowing their progress, further exacerbation or development, so as to appear later than in the absence of the treatment according to the invention.

The inhibitors of the invention and any composition thereof may be administered as a single daily dose or multiple daily doses, preferably, every 1 to 7 days. It is specifically contemplated that administration may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The treatment may last up to a day, two days, three days, four days, five days, six days, a week, two weeks, three weeks, four weeks, a month, two months three months or even more. Specifically, administration will last from one day to one month. Most specifically, administration will last from one day to 7 days.

Single or multiple administrations of the compositions of the invention are administered depending on the dosage and frequency as required and tolerated by the patient. In any event, the composition should provide a sufficient quantity of the biofilm formation inhibitor/s of the invention to effectively treat the patient. Preferably, the dosage is administered once but may be applied periodically until either a therapeutic result is achieved or until side effects warrant discontinuation of therapy. Generally, the dose is sufficient to treat or ameliorate symptoms or signs of disease without producing unacceptable toxicity to the patient.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment and diagnosis methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the composition/s and method/s of the invention are intended for mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof.

In certain embodiments, the method of the invention may optionally provide a combined treatment using the inhibitors of the invention and at least one anti-microbial agent, or in combination with said additional anti-microbial agent. The term “antimicrobial agent” as used herein refers to any entity with antimicrobial activity (either bactericidal or bacteriostatic), i.e. the ability to inhibit the growth and/or kill bacterium, for example Gram positive- and Gram negative bacteria. An antimicrobial agent may be any agent which results in inhibition of growth or reduction of viability of a bacteria by at least about 10%, 20%, 30% or at least about 40%, or at least about 50% or at least about 60% or at least about 70% or more than 70%, for example, 75%, 80%, 85%, 90%, 95%, 97%, 99%, 99.9%, 99.99%, 99.999%, 99.9999% or 100% or any integer between 30% and 99.9999% or more, as compared to in the absence of the antimicrobial agent. Stated another way, an antimicrobial agent is any agent which reduces a population of microbial cells, such as bacteria by at least about 30% or at least about 40%, or at least about 50% or at least about 60% or at least about 70%, 80, 90%, 95%, 97%, 99%, or more than 99%, or any integer between 30% and 99.9999% as compared to in the absence of the antimicrobial agent. In yet some further embodiments, reduction and inhibition of biofilm formation may be in log terms, in the range of 2 to 6, specifically, 2, 3, 4, 5, 6 log. More specifically, 3-4 log reduction when compared to biofilm formation in the absence of the inhibitors of the invention. In one embodiment, an antimicrobial agent is an agent which specifically targets a bacteria cell. In another embodiment, an antimicrobial agent modifies (i.e. inhibits or activates or increases) a pathway which is specifically expressed in bacterial cells. An antimicrobial agent can include any chemical, peptide (i.e. an antimicrobial peptide), peptidomimetic, entity or moiety, or analogues of hybrids thereof, including without limitation synthetic and naturally occurring non-proteinaceous entities. In some embodiments, an antimicrobial agent is a small molecule having a chemical moiety. For example, chemical moieties include unsubstituted or substituted alkyl, aromatic or heterocyclyl moieties including macrolides, leptomycins and related natural products or analogues thereof.

The invention therefor encompasses the option of combined treatment combining the inhibitors of the invention or any compositions thereof with an anti-microbial agent. Of particular interest for combined therapy may be the β lactam antibiotics. The term “β-lactam” or “β lactam antibiotics” as used herein refers to any antibiotic agent which contains a β-lactam ring in its molecular structure.

β-lactam antibiotics are a broad group of antibiotics that include different classes such as natural and semi-synthetic penicillins, clavulanic acid, carbapenems, penicillin derivatives (penams), cephalosporins (cephems), cephamycins and monobactams, that is, any antibiotic agent that contains a β-lactam ring in its molecular structure. They are the most widely-used group of antibiotics. While not true antibiotics, the β-lactamase inhibitors are often included in this group.

β-lactam antibiotics are analogues of D-alanyl-D-alanine the terminal amino acid residues on the precursor NAM/NAG-peptide subunits of the nascent peptidoglycan layer. The structural similarity between β-lactam antibiotics and D-alanyl-D-alanine prevents the final crosslinking (transpeptidation) of the nascent peptidoglycan layer, disrupting cell wall synthesis.

Generally, β-lactams are classified and grouped according to their core ring structures, where each group may be divided to different categories. The term “penam” is used to describe the core skeleton of a member of a penicillin antibiotic. i.e. a β-lactam containing a thiazolidine rings. Penicillins contain a β-lactam ring fused to a 5-membered ring, where one of the atoms in the ring is sulfur and the ring is fully saturated. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins include methicillin, dicloxacillin and flucloxacillin. The narrow spectrum β-lactamase-resistant penicillins may include temocillin. The moderate spectrum penicillins include for example, amoxicillin and ampicillin. The broad spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin.

As noted above, according to some embodiments, the inhibitors of the invention may be administered with or in combination with at least one additional therapeutic and anti-microbial or antibiotic agent. The term “in combination with” such as when used in reference to a therapeutic regimen, refers to administration or two or more therapies over the course of a treatment regimen, where the therapies may be administered together or separately, and, where used in reference to drugs, may be administered in the same or different formulations, by the same or different routes, and in the same or different dosage form type.

As noted above, the present invention involves the use of different active ingredients, for example, the inhibitors of the invention, specifically, the PstS-N-loop derived peptides and any fragments or derivatives thereof, and at least one anti-microbial or antibiotic agent that may be administered through different routes, dosages and combinations. More specifically, the treatment of infections associated with bacterial biofilm formation, as well as any diseases and conditions associated therewith, with a combination of active ingredients may involve separate administration of each active ingredient. Therefore, a kit providing a convenient modular format of the antagonist of the invention, specifically, the inhibitors of the invention and anti-microbial agents required for treatment would allow the required flexibility in the above parameters.

Thus, in another aspect, the invention provides a kit. More specifically, as encompassing the possibility of combined therapy and combined therapy regimen, the present invention further provides in certain embodiments thereof a kit comprising: (a) at least one of the inhibitors of the invention or any composition comprising the same, optionally in a first dosage form; and (b) at least one antibiotic agent, as discussed above, optionally in a second dosage form. The kit of the invention may facilitate combined treatment using different modes of administration for each compound as well as different duration of treatment.

In more specific embodiments, it should be appreciated that each of the multiple components of the kit may be administered simultaneously.

Alternatively, each of said multiple dosage forms may be administered sequentially in either order.

More specifically, the kits described herein can include a composition as described, or in separate multiple dosage unit forms, as an already prepared liquid topical, nasal or oral dosage form ready for administration or, alternatively, can include the composition as described as a solid pharmaceutical composition that can be reconstituted with a solvent to provide a liquid dosage form. When the kit includes a solid pharmaceutical composition that can be reconstituted with a solvent to provide a liquid dosage form (e.g., for oral administration), the kit may optionally include a reconstituting solvent. In this case, the constituting or reconstituting solvent is combined with the active ingredient to provide liquid dosage forms of each of the active ingredients or of a combination thereof. Typically, the active ingredients are soluble in so the solvent and forms a solution. The solvent can be, e.g., water, a non-aqueous liquid, or a combination of a non-aqueous component and an aqueous component. Suitable non-aqueous components include, but are not limited to oils, alcohols, such as ethanol, glycerin, and glycols, such as polyethylene glycol and propylene glycol. In some embodiments, the solvent is phosphate buffered saline (PBS).

Still further, as noted above, the present invention provides efficient methods and compositions for inhibiting bacterial biofilm formation. It should be therefore appreciated that in addition to therapeutic applications specified above, the invention further encompasses the option of preventing bacterial biofilm formation on different surfaces, solid or semi-solid supports or any other solid or semi-solid or liquid material or substance. Of particular interest are hospital surfaces.

The compositions and kits of the invention may be therefore formulated as a spray, a stick, paint, a gel, a cream, a wash, a liquid, a wipe, foam, soap, oil, a solution, a lotion, an ointment or a paste.

As noted above, this strategy may be applied for treating hospital surfaces and hand sanitizers soaps or other liquids for targeting the skin flora of medical personnel.

In some specific embodiments, the methods of the invention involve the steps of contacting a surface, specifically a solid or liquid surface, container, tube, article, or any substance (specifically, in the vicinity of the treated subject) with the inhibitors of the invention or any compositions or kits thereof.

As used herein the term “contacting” refers to the positioning of the inhibitors of the invention or any compositions or kits thereof such that they are in direct or indirect contact with the bacterial cells forming biofilm. Thus, the present invention contemplates both applying the inhibitors of the invention or any compositions or kits thereof to a desirable surface and/or directly to the bacterial cells.

Contacting surfaces with the inhibitors of the invention or any compositions or kits thereof can be effected using any method known in the art including spraying, spreading, wetting, immersing, dipping, painting, ultrasonic welding, welding, bonding or adhering.

The present invention envisages contacting a wide variety of surfaces with the inhibitors of the invention or any compositions or kits thereof including fabrics, fibers, foams, films, concretes, masonries, glass, metals, plastics, polymers, and like.

According to a particular embodiment, the inhibitors of the invention or any compositions or kits thereof are contacted with surfaces present in a hospital, hospice, old age home, or other such care facility.

Other surfaces related to health include the inner and outer aspects of those articles involved in water purification, water storage and water delivery, and those articles involved in food processing. Thus the present invention envisions coating a solid surface in a food or beverage factory.

Surfaces related to health can also include the inner and outer aspects of those household articles involved in providing for nutrition, sanitation or disease prevention. Thus, the inhibitors of the invention or any compositions or kits thereof may also be used for disinfecting toilet bowls, catheters, NG tubes, inhalators and the like.

In other embodiments, the inhibitors of the invention or any compositions or kits thereof may be applied in the vicinity of a treated subject. The expression “vicinity of the treated subject” relates to the perimeter surrounding said subject onto which the kit according to the invention may be applied in order to prevent bacterial biofilm formation. Therefore, it is understood that the “vicinity of said subject” encompasses all objects present within a range of up to at least about 1 centimeter (cm), 2 cm, 3 cm, 4 cm, 5 cm, 6 cm, 7 cm, 8 m, 9 m, 10 cm, 20 cm, 30 cm, 40 cm, 50 cm, 60 cm, 70 cm, 80 cm, 90 cm, 1 meter (m), 2 m, 3 m, 4 m, 5 m, 6 m, 7 m, 8 m, 9 m, 10 m, 11 m, 12 m, 13 m, 14 m, 15 m, 16 m, 17, m 18 m, 19 m, 20 m, 30 m, 40 m or even 50 m of said subject. The term “vicinity of said subject” also relates to objects to which the inhibitors of the invention or any compositions or kits thereof are applied to prior to their placement in said range of the treated subject.

Bacterial biofilm formation on contact lenses (CLs), and CL storage cases and care solutions may be a risk factor for CL-associated corneal infection and may explain the persistence of organisms in CL storage cases. Different types of lens wear modalities require the use of a contact lens storage case and care solutions for overnight storage and disinfection. However, the contact lens storage cases as well as storage solutions can become contaminated by bacteria and other pathogenic micro-organisms. Factors other than hygiene behaviors, including biofilm formation and microbial resistance, may be associated with persistent microbial contamination of contact lens storage cases and care solutions.

During storage the lenses are susceptible to colonization by a variety of bacterial strains and other microorganisms, and this problem exists even when the lenses are stored in a disinfecting solution containing hydrogen peroxide, chiorhexidine, biguanides or quaternary ammonium compounds. While the most serious infection associated with contact lens use may be microbial keratitis, contamination of the lens care system could lead to production of toxins that can affect the eye. Biofilms may form when bacterial cells attach to the interior surfaces of the lens case. By providing efficient inhibitors of biofilm formation, specifically, any of the peptides of the invention, specifically, the PstS-N-loop-derived peptides or any derivatives, enantiomers and combinations thereof, the invention further provides compositions and methods for storing contact lens, as well as methods for inhibiting, reducing or eliminating corneal infections. The methods described above may comprise the steps of providing a lens storage container coated with the biofilm inhibitors of the invention and alternatively or additionally, providing care solutions (storage solution) comprising the inhibitors of the invention, specifically, any of the PstS-N-loop-derived peptides of the invention, specifically, the peptides as denoted by SEQ ID NO. 23-30 and 56, and inserting the contact lens into the container coated with the inhibitors of the invention and/or or rinsing the contact lens with a solution comprising an effective amount of the inhibitors of the invention.

It should be further appreciated that the invention thus provides contact lenses storage case/s coated with, applied or containing the inhibitors of the invention. In yet some further embodiments, the invention provides contact lenses storage and care solutions containing the inhibitors of the invention.

Still further, indwelling medical devices including vascular catheters are becoming essential in the management of hospitalized patients by providing venous access. The benefit derived from these catheters as well as other types of catheters such as peritoneal catheters, cardiovascular, orthopedic and other prosthetic devices is often upset by infectious complications associated with bacterial biofilm formation.

Colonization of bacteria on the interior surfaces of the catheter or other part of the device can produce serious complications, including the need to remove and/or replace the implanted device and to vigorously treat secondary infective conditions.

By providing an effective tool for preventing and inhibiting biofilm formation, the present invention further encompasses the use of the inhibitors of the invention, specifically, the PstS-N-loop-derived peptides of SEQ ID NO. 23-30 and 56, or any derivatives, enantiomers or combinations thereof, in inhibiting, reducing, preventing or eliminating biofilm formation in medical devises and materials (solutions and solids). The inhibitors provided by the invention may be applied on surfaces of medical device or added to storage, lock or rinse solutions or solids used for medical applications.

The medical devices which are amenable to coating, rinsing, flushing or storing with the inhibitors of the invention generally have surfaces composed of thermoplastic or polymeric materials such as polyethylene, Dacron, nylon, polyesters, polytetrafluoroethylene, polyurethane, latex, silicone elastomers and the like. Devices with metallic surfaces are also amenable to coatings rinsing or storing with the inhibitors of the invention, or any solution or material comprising the same. Particular devices especially suited for application of the biofilm formation inhibitors of the invention include intravascular, peritoneal, pleural and urological catheters, heart valves, cardiac pacemakers, vascular shunts, and orthopedic, intraocular, or penile prosthesis.

Still further, small bore tubing that delivers ordinary running water, purified or not, to fixtures such as dental units, internal endoscopy tubing, catheter tubing, sterile filling ports, and tubing used for sterile manufacturing, food processing and the like, develop bacterial growth and biofilm formation on their interior surfaces, as is well known. It should be appreciated that the inhibitors of the invention may be applicable also for preventing and reducing biofilm formation in small bore tubing as discussed herein.

As noted above, the inhibitors of the present invention, specifically, any of the PstS-N-loop-derived peptides of the invention or any derivatives or combinations thereof, can be used to reduce or prevent biofilm formation on non-biological semi-solid or solid surfaces. Such a surface can be any surface that may be prone to biofilm formation and adhesion of bacteria. Non-limiting examples of surfaces include hard surfaces made from one or more of the following materials: metal, plastic, rubber, board, glass, wood, paper, concrete, rock, marble, gypsum and ceramic materials, such as porcelain, which optionally are coated, for example, with paint or enamel.

In certain embodiments, the surface is a surface that contacts with water or, in particular, with standing water. For example, the surface can be a surface of a plumbing system, industrial equipment, water condensate collectors, equipment used for sewer transport, water recirculation, paper pulping, and water processing and transport. Non-limiting examples include surfaces of drains, tubs, kitchen appliances, countertops, shower curtains, grout, toilets, industrial food and beverage production facilities, and flooring. Other surfaces include marine structures, such as boats, piers, oil platforms, water intake ports, sieves, and viewing ports, the hulls of ships, surfaces of docks or the inside of pipes in circulating or pass-through water systems. Other surfaces are susceptible to similar biofilm formation, for example walls exposed to rain water, walls of showers, roofs, gutters, pool areas, saunas, floors and walls exposed to damp environs such as basements or garages and even the housing of tools and outdoor furniture.

As noted above, the inhibitors of the invention, specifically, any of the PstS-N-loop-derived peptides described herein, can be applied to a surface by any known means, such as by covering, coating, contacting, associating with, filling, or loading the surface with an effective amount of the inhibitors of the invention. The inhibitors of the invention can be applied to the surface with a suitable carrier, e.g., a fluid carrier, that is removed, e.g., by evaporation, to leave a coating containing the inhibitors of the invention. In specific examples, the inhibitors of the invention may be directly affixed to a surface by either spraying the surface, by dipping the surface into or spin-coating onto the surface, for example with a solution containing the inhibitors of the invention, or by other covalent or non-covalent means. In other instances, the surface may be coated with an absorbent substance (such as a hydrogel) that absorbs the inhibitors of the invention. The inhibitors of the invention, specifically, any of the PstS-N-loop-derived peptides, more specifically, any of the peptides of SEQ ID NO. 23-30 and 56, or any derivatives, enantiomers or any combinations and compositions thereof, are suitable for treating surfaces in a hospital or medical setting. Application of the inhibitors of the invention, and compositions described herein can inhibit biofilm formation or reduce biofilm formation when applied as a coating, lubricant, storage, washing or cleaning solution, etc.

The inhibitors of the invention as described herein may be also suitable for treating, especially preserving, textile fiber materials. Such materials are undyed and dyed or printed fiber materials, e.g. of silk, wool, polyamide or polyurethanes, and especially cellulosic fiber materials of all kinds. Such fiber materials are, for example, natural cellulose fibers, such as cotton, linen, jute and hemp, as well as cellulose and regenerated cellulose. Paper, for example paper used for hygiene purposes, may also be provided with ant biofilm properties using one or more of the inhibitors of the invention, described herein. It is also possible for nonwovens, e.g. nappies/diapers, sanitary towels, panty liners, and cloths for hygiene and household uses, to be provided with ant biofilm properties.

The inhibitors of the invention, described herein are suitable also for treating, especially imparting ant biofilm properties to or preserving industrial formulations such as coatings, lubricants etc.

The inhibitors of the invention, specifically, any of the PstS-N-loop-derived peptides described herein can also be used in washing and cleaning formulations, e.g. in liquid or powder washing agents or softeners. The inhibitors of the invention, described herein can also be used in household and general-purpose cleaners for cleaning and disinfecting hard surfaces.

The inhibitors of the invention described herein can also be used for the ant biofilm treatment of wood and for the ant biofilm treatment of leather, the preserving of leather and the provision of leather with ant biofilm properties. The inhibitors of the invention described herein can also be used for the protection of cosmetic products and household products from microbial damage. The inhibitors of the invention described herein are useful in preventing bio-fouling, or eliminating or controlling microbe accumulation on the surfaces either by incorporating one or more of the inhibitors of the invention described herein into the article or surface of the article in question or by applying the inhibitors or any composition thereof to these surfaces as part of a coating or film. Such surfaces include surfaces in contact with marine environments (including fresh water, brackish water and salt water environments).

In yet some further embodiments, the substrate to be treated by the inhibitors of the invention can be an inorganic or organic substrate, for example, a metal or metal alloy, a thermoplastic, elastomeric, inherently cross-linked or cross-linked polymer as described above, a natural polymer such as wood or rubber; a ceramic material; glass; leather or other textile. The substrate may be, for example, non-metal inorganic surfaces such as silica, silicon dioxide, titanium oxides, aluminum oxides, iron oxides, carbon, silicon, various silicates and sol-gels, masonry, and composite materials such as fiberglass and plastic lumber (a blend of polymers and wood shavings, wood flour or other wood particles).

Still further, the inhibitors of the invention or any compositions or kits thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the inhibitors of the invention or any compositions or kits thereof may last up to a day, two days, three days, four days, five days, six days, a week, two weeks, three weeks, four weeks, a month, two months three months or even more. Specifically, application may last from one day to one month. Most specifically, application may last from one day to 7 days. In yet some other embodiments, application of the inhibitors of the invention or any compositions or kits thereof may be a routine procedure, specifically, daily procedure of treating surfaces, articles or any substance, for example, in a hospital environment.

Single or multiple applications of the inhibitors of the invention or any compositions or kits thereof are applied depending on the amount and frequency as required. In any event, the inhibitors of the invention or any compositions or kits thereof should provide a sufficient quantity to effectively prevent bacterial biofilm formation and most importantly, to prevent any pathologic disorder in a mammalian subject, caused by bacteria forming biofilm. Preferably, the effective amount may be applied once but may be applied periodically until a result is achieved.

The invention further provides a screening method for an antimicrobial compound that inhibits, reduces or eliminates bacterial biofilm formation, the method comprising:

a. obtaining a candidate compound that binds the N′ loop extension of PstS, any orthologs, or any fragment, variant, derivative, homologue and mutant thereof;
b. determining the effect of the compound selected in step (a), on bacterial biofilm formation. whereby inhibition of biofilm formation is indicative of the antimicrobial activity of said compound.

The candidate compound may be obtained by the steps of:

a. providing a mixture comprising said N′ loop extension of PstS, or any fragment, variant, derivative, homologue and mutant thereof;
b. contacting said mixture with said test candidate compound under suitable conditions for said binding; and
c. determining the effect of the test compound on an end-point indication, whereby modulation of said end point is indicative of binding of said N′ loop extension of PstS, or any fragment thereof to said test compound.

In some embodiments the candidate compounds may be provide using in silico screening using crystallography data.

In further embodiments, the candidate compound is evaluated by determining the ability of said compound to inhibit biofilm formation, using in non-limiting examples, the flow chamber assay described by the invention.

Still further, the invention provides any of the inhibitors described herein for use in a method for inhibiting, reducing or eliminating bacterial biofilm formation.

In yet a further aspect, the invention provides the use of any of the inhibitors of the invention in the preparation of a composition for inhibiting, reducing or eliminating bacterial biofilm formation.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al, (1989); “Current Protocols in Molecular Biology” Volumes I-III Ausubel, R. M., ed. (1994); Ausubel et al, “Current Protocols in Molecular Biology”, John Wiley and Sons, Baltimore, Md. (1989); Perbal, “A Practical Guide to Molecular Cloning”, John Wiley & Sons, New York (1988); Watson et al, “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Procedures

Protein Expression and Purification in E. coli

PA PstS and PstS mutants were expressed in E. coli Tuner strain (Novagen). Expression and purification procedures are detailed in a previous publication of the inventors [Neznansky and Opatowsky (3)]. In brief, transformed cells were grown in Terrific Broth media, and protein expression was induced with 200 μM IPTG over a 12 h period at 16° C. Periplasmic extraction was carried out immediately after cell harvest using a sucrose gradient. PstS proteins were further isolated using consecutive metal chelate and ion exchange chromatography. For crystallization, wild-type PstS was concentrated to 30 mg ml⁻¹, divided into aliquots, and flash-frozen in liquid N₂. A constant concentration of 5 mM NaPO₄ was maintained throughout the preparation of wild-type PstS designated for crystallization. For determination of binding constants, wild-type and mutant forms of PstS were first stripped from phosphate by a thorough wash with phosphate-free buffer (70 CVs) at the metal-chelate chromatography stage.

### Crystallization, Experimental Phasing, and Structure Determination

PstS was crystallized, as reported in the inventors previous publication [Neznansky and Opatowsky (3)], using 2.5 M Na malonate as precipitant and 0.1 M Tris pH=8. Diffraction data for the PstS crystals were measured on beamlines ID23 and ID29 at the ESRF and ID14.1 at BESSY II, and were processed and scaled to the best resolution of 1.89 Å using the XDSAPP software package. Molecular replacement attempts could not place more than one copy of the search model (PstS from Vibrio cholera, PDB code 1TWY). In order to obtain phase information, diffraction data were collected from crystals soaked in various heavy atoms, including 5-amino-2,4,6-triiodoisophthalic acid (I3C) and K₂PtCl₄; however; none could produce an independent structure solution. Ultimately, data from an I3C-soaked crystal was successfully used in an iterative process of molecular replacement using the BALBES server and SAD (Phenix), which placed all four PA PstS molecules in the asymmetric unit. Refinement was performed using Phenix and the ReDo server. Data collection and model statistics are summarized in Table 1.

### Peptides

Synthetic peptides 1-6 (as denoted by SEQ ID NO. 25-30, respectively) were synthesized by/purchased from Synpeptide. Co. Ltd.

TABLE 1

| Crystal form | Form-1 | Form-2 |
|---|---|---|
| Crystallization precipitant | Sodium Malonate | PEG 8000 |
| Wavelength (Å) | 0.9508 | 0.9763 |
| Resolution range (Å) | 122.2-1.89 (1.95-1.89) | 62.16-1.855 (1.921-1.855) |
| Space group | P 21 21 21 | C 2 2 21 |
| Unit cell | 35.4 148.1 216.4 90 90 90 | 67.5 151.3 109 90 90 90 |
| Total reflections | 1331276 (134370) | 94280 (9359) |
| Unique reflections | 92943 (9183) | 47406 (4684) |
| Multiplicity | 14.3 (14.6) | 2.0 (2.0) |
| Completeness (%) | 99.93 (100) | 99.59 (99.94) |
| Mean I/sigma (I) | 21.56 (6.57) | 5.08 (1.14) |
| Wilson B-factor | 20.79 | 24.67 |
| R-merge | 0.10 (0.6) | 0.09749 (0.7043) |
| R-meas | 0.105 | 0.1379 |
| CC1/2 | 0.999 (0.944) | 0.993 (0.205) |
| CC* | 1 (0.086) | 0.998 (0.583) |
| R-work | 0.1902 (0.2051) | 0.2160 (0.3879) |
| R-free | 0.2157 (0.2435) | 0.2625 (0.4088) |
| No. of non-hydrogen atoms | 9495 | 4490 |
| Macromolecules | 9024 | 4081 |
| Ligands | 24 | 10 |
| Water | 447 | 399 |
| Protein residues | 1192 | 539 |
| RMS (bonds) | 0.014 | 0.009 |
| RMS (angles) | 1.57 | 1.24 |
| Ramachandran favored (%) | 99 | 98 |
| Ramachandran outliers (%) | 0 | 0 |
| Clashscore | 1.65 | 7.65 |
| Average B-factor | 32.50 | 33.6 |
| Macromolecules | 32.50 | 33.10 |
| Ligands | 33.10 | 22.00 |
| Solvent | 33.10 | 39.90 |

Values in parentheses indicate the specific values in the particular highest resolution shell. Rmerge=ΣhklΣi|Ii(hkl)−<Ii(hkl)>|/ΣhklΣiIi<(hkl)>, where the sum i is over all separate measurements of the unique reflection hkl. Rwork=Σhkl∥Fobs|−|Fcalc∥Σhkl|Fobs|. Rfree was calculated as Rwork, but summed over a 5% test set of randomly selected reflections. CC1/2 is the correlation of random one half of the observations to the other half. *AcKt1 blocked Kv1.3 with an 1050 value of 395 nM.

### Molecular Graphics and Structure Deposition

Molecular images were produced using PyMOL (The PyMOL Molecular Graphics System, Version 1.8 Schrodinger, LLC.) The atomic coordinates and structure factors were deposited in the protein data bank (PDB) with the identification codes 40 MB and 4PQJ. Similarity model alignments were generated using DaliLite (European Bioinformatics Institute, Hinxton, UK; and the universal similarity metric (USM). Ligplot (European Bioinformatics Institute) was used to generate 2D interaction schemes. GraphPad prism software (GraphPad, San Diego, Calif., USA) was used in binding affinity calculations.

### PAO1 Bacterial Strains, Plasmids, and Media

The bacterial strains and plasmids used in this study are shown in Table 2.

TABLE 2
Strains used in this study

| | | |
|---|---|---|
| Strain or | | Source or |
| plasmid | Description | reference |

PA strains

| | | |
|---|---|---|
| PAO1 | Wild type | (9) |
| ΔpstS | PAO1 with an unmarked deletion of pstS | (10) |

E. coli strains

| | | |
|---|---|---|
| DH5α | F′/endA1 hsdR17 supE44 thi-1 recA1 gyrA | (11) |
| | relA1 Δ(lacZYA-argF) U169 deoR (Φ80 dlacZ- | |
| | M15 recA1) | |
| S17.1 (λpir) | recA derivative of E. coli 294 (F-thi pro hsdR) | Y. Irie and M. R. Parsek |
| | carrying a | |
| | modified derivative of IncPα plasmid pRP4 | |
| | (Ap^{s} Tc^{s} Km^{s}) | |
| | integrated in the chromosome, Tp^{r}; lysogenized | |
| | with bacteriophage λpir | |
| T7-express | Chemically competent BL21 E. coli phage- | NEB |
| | resistant cells suitable for transformation and | |
| | protein expression | |

Plasmids

| | | |
|---|---|---|
| pUCP18Ap | A broad-host range cloning vector. Cb^{R}/Amp^{R} | (12) |
| DB3.1 | pEX18Gm containing the Gateway (GW) | Nan Fulcher and |
| pEX18GmGW | destination cloning site. GmR | Matthew Wolfgang |
| pIBK1238 | pUCP18Ap containing a His-tagged pstS gene | This study |
| pIBK1195 | pUCP18Ap containing a His-tagged pstS gene | This study |
| | with a S96E point mutation | |
| pIBK1196 | pUCP18Ap containing a His-tagged pstS gene | This study |
| | with a 12 aa deletion in the C-terminus | |
| pIBK1491 | pUCP18Ap containing a His-tagged pstS gene | This study |
| | with a 13 aa deletion in the N-terminus | |
| pIBK1662 | pUCP18Ap containing the N-terminal region of | This study |
| | pstS | |
| | (aa 1-38) | |
| pET22b(+) | Cloning vector that contains N-terminal pelB | |
| | signal for potential periplasmic localization and | |
| | C-terminal His-tag | |

For a high phosphate level was used M9 minimal medium (20 mM NH₄Cl, 12 mM Na₂HPO₄, 22 mM KH₂PO₄, 8.6 mM NaCl, 1 mM MgSO₄, 1 mM CaCl₂, and 11 mM dextrose) supplemented with 50 μM FeCl₃. For alkaline phosphatase assay, strains were grown on M9 containing one fifth of the standard phosphate concentration (2.4 mM Na₂HPO₄ and 4.4 mM KH₂PO₄), supplemented with 50 μM FeCl₃. For swarming assays was used M9 minimal medium or M9 depleted of phosphate (20 mM NH₄Cl; 8.6 mM NaCl; 1 mM MgSO₄; 1 mM CaCl₂; 11 mM Dextrose), both supplemented with 0.5% Casamino acids, 50 μM FeCl₃ and solidified with 0.5% Bacto Agar (Difco). For generating the pstS knockout, Luria-Bertani broth (LB, Difco), No Salt LB (NSLB, 1% tryptone, 0.5% yeast extract), Vogel Bonner Minimal Medium (VBMM), and Psuedomonas Isolation Agar (PIA, Difco) were used. All strains were grown at 37° C. with shaking, unless specified otherwise. The antibiotic concentrations used in this study were 300 μg/ml or 150 μg/ml carbenicillin for PA and 100 μg/ml ampicillin for E. coli.

### Construction of Strains and Plasmids for PAO1 Expression

The pstS deletion mutant was constructed as previously described (13). Overlap extension PCR using the primers specified in Table 3 was used in order to generate a fragment containing the upstream and downstream regions of pstS, and cloned into the allelic exchange vector DB3.1 pEX18GmGW using BP-Clonase (Invitrogen). The deletion was introduced to PAO1 using biparental mating (6) and generated using a standard method for a two-step allelic exchange (14), and further confirmed by PCR. Overlap extension PCR using the primers specified in Table 3 was used in order to generate all of the structural mutations. The PCR product was cloned into pUCP18Ap using T4 Ligase (Thermo). Constructs were verified by sequencing and electroporated into the ΔpstS strain.

More specifically, the S96E point mutation was constructed using overlap extension PCR with the primers of SEQ ID. NOs. 37 and 38, as shown in Table 3. The PCR product was digested with EcoRI and HindIII and cloned into an EcoRI and HindIII-digested pUCP18Ap using T4 Ligase (Thermo Scientific). The amino acid sequence of the S96E PstS mutant is denoted by SEQ ID NO. 52.

The N-terminus deletion was constructed using the primers of SEQ ID. NOs. 39, 40 and 41, as shown in Table 3. Then, the PCR product was digested with EcoRI and AflIII (Thermo Scientific), and cloned into an EcoRI and AflIII-digested pUCP18Ap using T4 Ligase (Thermo Scientific). The amino acid sequence of the cloned N-terminus deleted PstS construct is denoted by SEQ ID NO. 53.

The vector expressing only the N-terminus sequence of PstS was constructed using the primers of SEQ ID NO. 54 and 55, shown in Table 3 below. The PCR product was cloned into pUCP18Ap. The amino acid sequence of the cloned N-terminus PstS construct is denoted by SEQ ID NO. 50.

TABLE 3
Primers used in this study

| | Sequence | | |
|---|---|---|---|
| | (5′ to 3′) and | Under- | |
| Primer | SEQ. ID. NOs. | lined | Used for |
| PstSUpF01- | GGGGACAAGTTTGTAC | | pstS |
| GWB1 | AAAAAAGCAGGCTCAC | | knockout |
| | AATTGCCCTGGAAACT | | |
| | ACC; | | |
| | SEQ. ID. NO. 31 | | |
| PstSUpR01 | TACAGGCCCAGTTCCT | | pstS |
| | TGATCGCCGGCCGCCA | | knockout |
| | TCAAACGCTT; | | |
| | SEQ. ID. NO. 32 | | |
| PstSDownF01 | GGCGATCAAGGAACTG | | pstS |
| | GG; | | knockout |
| | SEQ. ID. NO. 33 | | |
| PstSDownR01- | GGGGACCACTTTGTAC | | pstS |
| GWB2 | AAGAAAGCTGGGTACG | | knockout |
| | ACCAGCACGTACCAG; | | |
| | SEQ. ID. NO. 34 | | |
| 1320 | AGAGGAATTCTAAGGA | EcoRI | Cloning of |
| | GGAATAACATATGAAA | site | pstS + |
| | CTCAAGCGTTTGATG; | | His tag |
| | SEQ. ID. NO. 35 | | |
| 1322 | AGAGAAGCTTTTAGTG | HindIII | Cloning of |
| | ATGGTGATGGTGATGC | site | pstS + |
| | TCCAGGGCGGCGGCCA | | His tag |
| | GGCCCAGTTCCTTGAT; | | |
| | SEQ. ID. NO. 36 | | |
| Ser96US | AACCTGGGCCCGATGG | S96E | Point |
| | AACGCAAGATGAAGGA; | mutation | mutation |
| | SEQ. ID. NO. 37 | | |
| Ser96DS | GTCCTTCATCTTGCGTT | S96E | Point |
| | CCATCGGGCCCAGGTT; | mutation | mutation |
| | SEQ. ID. NO. 38 | | |
| 1325 | AGAGACATGTTCTTTC | AflII | PstS |
| | CTGCGTTATCCCCTG; | site | lacking |
| | SEQ. ID. NO. 39 | | 13 aa from |
| | | | the N- |
| | | | terminus |
| 1326 | CGGGCAACCTGTCGAG | | PstS |
| | C; | | lacking |
| | SEQ. ID. NO. 40 | | 13 aa from |
| | | | the N- |
| | | | terminus |
| 1327 | GCTCGACAGGTTGCCC | | PstS |
| | GCGCGGCTACCGCGGA | | lacking |
| | AG; | | 13 aa from |
| | SEQ. ID. NO. 41 | | the N- |
| | | | terminus |
| 010-YO | ATATGAATTCGGCGAT | EcoRI | pstS |
| | CGACCCGGCGCT; | site | pET22 |
| | SEQ. ID. NO. 42 | | cloning |
| 004-YO | GCGCAAGCTTCAGGCC | HindIII | pstS |
| | CAGTTCCTTGATCGC; | site | pET22 |
| | SEQ. ID. NO. 43 | | cloning |
| 257-YO | AACCTGGGCCCGATGG | S96E | Point |
| | AACGCAAGATGAAGGA | mutation | mutation |
| | C; | | |
| | SEQ. ID. NO. | | |
| | SEQ. ID. NO. 44 | | |
| 258-YO | GTCCTTCATCTTGCGTT | S96E | Point |
| | CCATCGGGCCCAGGTT; | mutation | mutation |
| | SEQ. ID. NO. 45 | | |
| 264-YO | ATATGAATTCGGTGTC | EcoRI | PstS delN′ |
| | GGGCAACCTGTCG; | site | |
| | SEQ. ID. NO. 46 | | |
| 1785 | TAAAAGCTTGGCACTG | | The N- |
| | GCCGT, | | Terminus |
| | SEQ. ID. NO. 54 | | of PstS |
| | | | aa (1-38) |
| 1786 | ACCGCTGGCTTTCTGAT | | The N- |
| | ATTC, | | Terminus |
| | SEQ. ID. NO. 55 | | of PstS |
| | | | aa (1-38) |

### Determination of Pi Binding Constants

For P³² binding assays, purified PstS wild-type and mutant proteins were incubated with 300 μl bed volume of talon beads (Clontech) in buffer containing 150 mM NaCl and 20 mM HEPES (pH=7.5) for 120 min in RT. The protein-bound beads were then washed and resuspended to a final volume of 900 μl, at which time the protein concentration was 1.93 nM (total 11.6 pmol). For each measuring point, 20 μl of suspended PstS-bound beads were mixed with 180 μl of solution with a range of phosphate concentrations (0.125-10 μM) premixed with P³², with specific activity of 285.6 Ci/mg (9139.2 Ci/mmol). Nonspecific binding was determined by using the same amounts of P³² in the presence of 0.125-10 mM non-radioactive phosphate. Phosphate binding was performed at RT for 30 min. Unbound phosphate was removed by centrifugation, and rapid wash of the beads was performed at 700 g for 5 min. PstS proteins were then eluted from the beads by buffer containing 150 mM NaCl, 20 mM HEPES (pH=7.5), and 500 mM imidazole. Scintillation liquid was added, and the amount of bound Pi was determined by a packard tri-carb liquid scintillation counter.

### Alkaline Phosphatase Assay

Alkaline phosphatase (AP) expression is enhanced under phosphate starvation (15). AP activity assay was therefore utilized to assess a strain's phosphate starvation levels. AP activity was measured by sampling strains grown in a liquid culture. Strains were grown overnight in M9 medium supplemented with FeCl₃ and carbenicillin. Afterwards, bacteria were diluted to an O.D595ₙₘ of 0.04 into 50 ml of fresh M9 medium with a fifth of the standard phosphate concentration, and supplemented with 50 μM FeCl₃. Strains were grown for an additional 24 h, then 15 ml from each strain was centrifuged for 10 min at 2,200 g (Centrifuge 5418, Eppendorf. The pellet was resuspended with 50 μl of chloroform. After 15 min of incubation at room temperature, 50 μl of 0.01 M Tris-HCl (pH=8) was added to each sample, and the samples were centrifuged for 20 min at 6,000 g. Afterwards, 30 μl from each sample's supernatant were added to a 96-well plate containing the reaction buffer [5 μl of 0.5 mM MgCl₂ and 10 μl of 1 M Tris (pH=9.5)]. Then, 5 μl of 500 mM p-nitrophenyl phosphate (PNPP, NEB) was added to each well and the reaction was read at 405 nm in an ELISA plate reader (Synergy™ 2 multi-detection microplate reader, Biotech). Results were normalized to each sample's total protein concentration using the Bradford assay (Thermo Scientific).

### Swarming Motility Assay

Strains were grown overnight in M9 medium supplemented with Casamino acids, FeCl₃, and carbenicillin. Afterwards, bacteria were diluted 1:10 into a similar fresh medium and grown for an additional three hours in order to reach logarithmic growth phase. An amount of 2.5 μl from each culture was plated in the middle of a swarming plate (see medium details above). Plates were incubated at 37° C. for 24 h.

### Flow Chamber Biofilm Experiment

The flow chamber system was designed to examine biofilm formation and was constructed as previously described (16). Bacteria were grown overnight in tryptic soy broth (TSB), then diluted to an OD of 0.15 into 1% TSB (Difco). The fresh bacterial culture was injected into the flow chamber using a sterile syringe and incubated for one hour to allow adhesion. Afterwards, the chamber was connected to the flow cell system and the pump was set to 2 rpm. The experiment was done at 37° C. After 72 h, bacteria were stained with Syto-9, and images were taken using Lecia TCS SPE confocal laser scanning microscope (CLSM). The excitation and emission wavelengths were 488 nm and 500-530 nm, respectively. Images (at least ten) were processed using Imaris analysis software, and biofilm quantification was done using PHLIP.

For flow cell biofilm experiments in the presence of the inhibitory peptides of the invention, the following specific protocol was used:

Bacterial strains (Pseudomonas aeruginosa strains PA14 and DK2) containing the plasmid pUCP-GFP inserted to bacterial cells by electroporation (pUCP18 that constitutionally expresses the GFP under the lac promoter), were inoculated from −80° C. stock into 2 mL TSB containing 300 μg/mL Carb (to maintain the pUCP-GFP plasmid) and grown overnight at 37° C. with agitation. The next day, bacteria were diluted to 0.05 OD (595 nm) in 1% TSB containing the antibiotics and loaded onto 6 channel 1μ-Slide I^{0.4} Luer uncoated (Ibidi). The slide was connected to a flow cell containing fresh 1% TSB with 0.05 Mm, 0.1 mM peptides or without peptides. Fresh media flowed through the slide by a pump at 10 mL/h (2 rpm). Bacteria were grown in the slide for 48 hours at 37° C. After 48 hours, 3 representative pictures from the beginning and the middle of the slide, were taken using SP8 confocal HyD microscope (Leica). Pictures were analyzed by Imaris software (version 7.2.2).

### Ectopic Expression Functional Studies

Examining the impact of ectopic expression of the N-terminus of PstS on PA01 wild type in flow cells was carried out using the flow cell biofilm model. The system consists of a 2 L media bottle containing 1% TSB, a peristaltic pump that supplies the nutrients in the media bottle in a constant rate, a bubble trap, a flow chamber in which the bacteria forms biofilm and a waste bucket, to which the media and bacterial waste is drained. The system is connected by silicone tubing. Bacteria were grown overnight in TSB, then diluted to an O.D of 0.15 into 1% TSB. The fresh bacterial culture was injected into the flow chamber using a sterile syringe and incubated for one hour to allow bacteria to attach to the glass surface. Afterwards, the chamber was connected to the flow cell system and the pump was set to 2 rpm (approx. 10 ml/h). The experiment was done at 37° C., and images were taken using Leica TCS SPE CLSM (Confocal Laser Scanning Microscope). Biofilm formation of PAO1, PAO1 carrying the vector over expressing PstS N-terminus region and ΔpstS (served as a control for low biofilm formation) were grown for 72 h, following which the biofilms were stained with Syto- and imaged using confocal microscopy. The excitation and emission wavelengths used were 488 and 500-530, respectively. Images were taken in sections of 0.71 μm, processed using Imaris analysis software and biofilm biovolume quantification was done using the ImageJ, PHLIP and MATLAB software's.

### N′ Loop Derived Peptides—Functional Studies

The static biofilm model was used to assess the ability of N′-loop derived peptides to reduce PA biofilm formation. Briefly, PA wild type bacteria were grown over night in M9 medium (20 mM NH₄Cl; 12 mM Na₂HPO₄; 22 mM KH₂PO₄; 8.6 mM NaCl; 1 mM MgSO₄; 1 mM CaCl₂; 11 mM Dextrose) at 37 C. Following growth cells were diluted to a final concentration of 5*10⁷ in M9 medium contacting 1/10 the phosphate concentration. 100 microliter of the bacterial suspension was transferred to each well of 96 wells plate. The six tested peptides were added at different concentrations 0.01, 0.05 and 0.1 mM. The plate was then incubated for 24 h at 37° C. to allow biofilm development. After incubation the medium was removed and the wells were carefully washed twice with sterile ddsH₂O to remove planktonic bacteria. Next 150 microliter of 1% crystal violet (w/v) was added to each well in order to stain the biofilm cells. The stain was allowed to incubate for 15 min after which the wells were washed again with ddsH₂O. The stain attached to the biofilm was then extracted by adding 200 microliter ethanol (95%). The biofilm biomass was quantified by reading the absorbance at 595ₙₘ using an ELISA plate reader.

### Confocal Microscopy

Leica TCS SPE CLSM (Confocal Laser Scanning Microscope) was used as described above.

### Statistical Analysis

Statistical analysis was carried out using unpaired t-test and Tukey's post-hoc test. P<0.05 was considered statistically significant.

### Example 1

### PstS Crystal Structure and Similarity to Orthologs

PstS was crystallized, as previously reported by the inventors [Neznansky and Opatowsky (3)] in two crystal forms: form-1 was crystallized under sodium malonate conditions with a P2₁2₁2₁ space group, and form-2 was crystallized under PEG 3350 conditions with a C222₁ space group. There are four PstS copies in the asymmetric unit of form-1 and two copies in that of form-2. Structural analysis and electron density map revealed that all copies have high backbone and side-chain similarity to each other (r.m.s.d of 0.443 Å), with a virtually identical ligand binding pocket fully occupied by one unsolvated PO₄ (FIG. 1A and FIG. 3A). Two regions were not visible in the form-2 C222₁ crystals. The first is the entire N′ loop, and the other—that spans the residues that form helix 8 and includes residues 245-260 (FIGS. 2A and B) of PA PstS amino acid sequence as denoted by SEQ ID NO. 49 [P. aeruginosa PstS—accession number NP_254056.1]], as denoted by SEQ ID NO. 47. PstS was classified to cluster D-III of the substrate binding protein (SBP) superfamily according to the classification presented by Berntsson et al. (4). Cluster D-III includes SBPs that bind tetrahedral oxyanions, e.g., molybdate, sulfate, and phosphate. Structural analysis revealed that PstS, like all cluster D members, consists of two globular domains, designated domain I and domain II, which are connected by a two-strand hinge (FIGS. 1A-1B). The two domains have a similar globular structure that includes a beta sheet core surrounded by peripheral alpha helixes. In PA PstS, the phosphate binding site is located at the cleft formed between the two domains, at a minimal distance of 10 Å from the exposed solvent surface. Further, the inventors compared the structures of the PA PstS and the molybdate binding protein (r.m.s.d. 2.9 Å), crystallized in a complex with the entire molybdate transporter (PDB 2ONK) (17). This analysis implicated that the presumed permease binding surface of PstS includes strands 1, 2, and helix 2 from domain I and strands 5 and 7 from domain II (FIG. 1A).

In further similarity studies, the inventors compared the structure of PA PstS to all PstS orthologs that have PDB-available structures. Most surprisingly, PA PstS exhibited the highest structural homology to PstS structures of several Gram-positive bacteria, and a lower homology to the more phylogenetically related Gram-negative PstS orthologs (FIG. 1C). For example, the PstS crystal structures of E. coli (PDB code 1IXH) and PA align poorly with r.m.s.d score of 2.9 Å (FIG. 2B). More specifically, the N′ loop extension that appears in PA PstS seemed to be a common feature among the Gram-positive bacterial orthologs and was generally lacking in Gram-negative PstSs.

### Example 2

### Design of Mutants Defective Only in One Activity

Based on the analysis of PA PstS crystal structures, the inventors designed mutations that should preserve the overall protein fold and integrity while (i) abrogating phosphate binding or (ii) eliminating putative biofilm-related functions of the N′ loop. The S96E mutation was designed to block phosphate binding by virtue of steric hindrance and electrostatic repulsion (FIGS. 3A-3B). The inventors hypothesized that while the absence of phosphate would result in some increased flexibility between the relative orientations of the two lobes, it would not alter the overall tertiary structure of the protein. In order to eliminate the N′ loop, the 25-AIDPALPEYQKASG-38 (also denoted by SEQ ID NO. 48) sequence was deleted. Further, the wild-type PstS and PstS mutants S96E and delN′ were expressed in E. coli using the pET 22b+ periplasmic E. coli expression vector and isolated by consecutive metal chelate and ion exchange chromatography before being analyzed using a superdex 200 10/300 gel-filtration column. The elution profile and volume was consistent with monomeric protein arrangements (estimated molecular weights in kDa units are marked by arrows) and indicate well-folded proteins (FIG. 4). Since both mutants, S96E and delN′, exhibited periplasmic expression yields and migration properties in size-exclusion chromatography similar to the wild-type PA PstS, it could be concluded that overall protein fold and integrity were indeed preserved.

### Example 3

### PstS Phosphate Binding

The above studies suggested that PO₄ is tightly held by ten amino acids from the two PstS domains that mediate most of the intra-domain contacts within PA PstS (FIG. 3B). Nine of these residues interact with the four phosphate oxygens through a total number of 13-14 hydrogen bonds (depending on distance criteria), while the tenth residue, Gly77, is engaged in hydrophobic interactions only.

For determining the binding constants of PO₄, phosphate-free PA PstS proteins, wild-type and mutant, were supplemented with P³²-labeled phosphate at pH 7.5 (FIG. 3C). The calculated dissociation constant (K_{D}) value for wild-type PstS was 0.84±0.12 μM, that is stronger binding than reported for the PstS orthologs from E. coli (˜3 μM) and M. tuberculosis (˜13 μM) measured under similar pH conditions (18). The delN′ mutant exhibited a K_{D} value (0.53±0.16 μM) similar to the wild-type protein, whereas S96E exhibited very weak or no binding.

To corroborate these biochemical results, the inventor measured phosphate starvation responses of PA wild-type and ΔpstS mutant bacteria complemented with each of the PA proteins (i.e., wild-type PstS, S96E, or delN′). These studies showed that complementation of ΔpstS mutant with S96E did not impact the phosphate starvation response, and the strain exhibited an alkaline phosphatase activity similar to ΔpstS, complemented with an empty vector (FIG. 3D). In contrast, complementation of ΔpstS mutant with wild-type PstS or delN′ resulted in reduced alkaline phosphatase activity similar to that measured in wild type.

The inventors further examined the impact of the PstS S96E mutation on swarming motility, a phenotype known to be induced under phosphate starvation. These studies showed that wild-type and ΔpstS bacteria complemented with either wild-type PstS or delN′ exhibited a hyper-swarming phenotype only under phosphate limitation (FIGS. 5A-5B, 5E-5F and 5I-5J). In contrast, ΔpstS (FIGS. 5C-5D) and ΔpstS complemented with the S96E mutant (FIGS. 5G-5H) exhibited a hyper-swarming phenotype even when phosphate concentrations were not limiting. Taken together, the affinity measurements, alkaline phosphatase activity, and swarming assay establish that the S96E mutation prevents effective phosphate binding and uptake in PA.

### Example 4

### The Interactions of the N′ Loop in PstS

The construct that was used for crystallography included the entire PA PstS sequence except for the amino-terminal 24-residue-long signal peptide. The pET22b+PelB peptide directs the PstS to enter the periplasm and is cleaved during that process. Notably, in the form-1 crystals (but not in form-2), virtually all the 298 residues of all four copies are clearly visible in the electron density map including the fourteen amino acids of the N′ loop that are not part of the canonical SBP fold (FIG. 1C). The N′ loop is engaged in intra- and inter-molecular interactions, the latter with symmetry residues in the crystal lattice. Intra-molecular contacts include hydrophobic interactions and hydrogen bonds within a shallow cleft formed between helixes 9 and 10 of domain I that opposes the putative permease binding surface (FIG. 6A). This analysis suggested that the N′ loop extension does not regulate PstS binding to the transmembrane permease, and that the N′ loop is further engaged in several intermolecular crystal-lattice contacts, such as the interaction with the loop connecting strands 8 and 9 in domain II (FIG. 6B). In sharp contrast to the ordered arrangement of the N′ loop in form-1, the loop is not visible in the two copies of form-2. This structural difference may be explained by an experimental side effect, such as different crystallization conditions or, rather, may represent a genuine property of the N′ loop, whereby different conformation states have functional implications. In summary, the above analyses of the crystal structure of PstS show that the symmetry inter-related PstS molecules are arranged in a fibrous-like arrangement thought N′-loop contacts.

### Example 5

### Significance of the Amino Terminal (N′) Loop of PstS in Biofilm Formation

Further, the inventors hypothesized that N′ loop truncation may affect the ability of PA to form biofilms. To which end, they compared the biofilm formation capacity of wild-type, ΔpstS, and delN′ mutant bacteria using the flow chamber biofilm assay (FIG. 7). The delN′ mutant bacteria were observed to exhibit a 62% decrease in biofilm biovolume, as compared to the same bacteria complemented with PstS. Complete elimination of PstS (ΔpstS) resulted in a 74% decrease. Confocal microscope images of bacterial biofilms produced by the wild-type PA and PA PstS deletion mutants under above conditions provided further support to the role of PstS in biofilm formation (FIG. 8). All together these studies suggested that the N′ loop plays a critical role in the ability of PA to form biofilms.

### Example 6

### The Ability of PstS to Promote Biofilm Formation is not Dependent on Phosphate Uptake

The inventors further determined if the two activities mediated by PA PstS, i.e., phosphate uptake and biofilm formation, are interdependent. To that end, they examined the performances of the delN′ and S96E mutants under the plate-swarming and flow-chamber biofilm assays, respectively. It was found that delN′ mutant bacteria behave virtually identically to the wild-type and ΔpstS-complemented delN′ mutant bacteria regarding phosphate binding and uptake (FIGS. 3C-3D) as well as swarming (FIGS. 5I-5J). Conversely, the S96E mutant, which is completely deficient in phosphate uptake, was observed to produce more biofilm than the delN′ strain, reaching levels comparable to wild-type bacteria (FIG. 7). These results demonstrated that the two activities mediated by PA PstS can indeed be separated.

### Example 7

### Ectopic Expression of the N′-Loop Reduces Biofilm Formation in the Wild-Type PA

The inventors further evaluated the potential of targeting the N′-loop as an anti-biofilm treatment. To which end, they constructed a vector constitutively expressing the PstS N′-loop (along with the native signal peptide, required for periplasmic targeting) and compared biofilm formation of the wild-type strain carrying an empty vector to the wild-type strain carrying N′-loop expressing vector (using the flow chamber biofilm assay as described in EXAMPLE 5). The results show that ectopic expression dramatically reduced biofilm formation, similarly to the pstS knock-out mutant (FIG. 9).

### Example 8

### N′-Loop Derived Peptides Reduces PA Biofilm Formation

To further evaluate if the N′-loop can serve as an anti-biofilm target, the inventors examined the effect of six different chemically synthesized N′-loop peptides on PA biofilm formation, as described above in Experimental procedures. Structural properties of these peptides and their relative effects on biofilm formation are demonstrated in FIG. 10. These results show a dose dependent and significant anti-biofilm activity, specifically in peptides that include the first eight amino acids of the N′-loop (peptides 1 to 3, FIG. 10B). More specifically, confocal microscope images of FIG. 10B clearly showed that peptide-3 (as denoted by SEQ ID NO. 27) inhibits most effectively biofilm formation. As shown in FIG. 10C, a modified peptide-3, specifically, an enantiomer of peptide-3 having the N-terminal Ala and C-terminal Glu residues in the D-form (as denoted by SEQ ID NO. 56), efficiently inhibited biofilm formation. It should be noted that this enantiomer derivation may exhibit enhanced stability and resistance to proteolytic degradation.

The inventors have further examined the effect of the D-enantiomer peptide-3 of the invention on clinical isolates. Therefore, PA14 and the clinical isolate DK2 (both express Plasmid GFP) were used as described above, and compared with the laboratory isolate, PA01 (that express genomic GFP). As shown in FIG. 11A, Addition of 0.1 mM D-peptide 3 to the media significantly reduces the biofilm formation in the different strains of P. aeruginosa. Confocal microscope images of FIG. 11B clearly showed that addition of the D-peptide 3 enantiomer (SEQ ID NO. 56) changes the biofilm formation phenotype of all PA strains examined, exhibiting a marked effect on both clinical isolates.

Apart from providing yet another confirmation to the central role of the N′-loop in biofilm formation in laboratory as well as in clinical isolates, these results highlight the potential to inhibit or compete with the N′-loop as an anti-biofilm strategy.

### Example 9

### In Vivo Efficacy Study of the Biofilm Formation Inhibitors

To evaluate the in vivo efficacy of then inhibitors of the invention, specifically the peptides as described herein, the inventors use mouse infection model. Briefly, C57/BL6 mice are intranasally infected with 3×10⁷ colony forming units (CFU) of P. aeruginosa PAO1 strain and/or wild-type P. aeruginosa strain and intravenously treated with the tested peptides. Mice are scored for viability throughout the infection. At day 4 and day 7 of infection, mice are sacrificed and lung tissues are homogenized in PBS buffer containing soybean trypsin inhibitor in order to determine the bacterial load. For the bacterial counts, 50 μl dilutions of the homogenate are plated on trypticase soy agar plates and then incubated for 24 hrs at 37° C. A group of animals that are not infected and one group that is not infected and treated with the peptide are served as further controls. In addition to CFU, a complete hematology screening of the blood samples and histology of lung tissue is performed to provide additional markers for the infection severity.

### Example 10

### In Vivo Efficacy Study of the Biofilm Formation Inhibitors Using the Implant Infection Model

In order to further evaluate the anti-biofilm activity of the peptides an implant infection model is utilized. Overnight cultures of Pseudomonas aeruginosa PAO1 are diluted to 10⁷ cell/ml in Tryptic Soy Broth. Two such solutions are prepared one without and with modified peptide-3 (as denoted by SEQ ID NO. 56) at a final concentration of 1 mg/ml. After which 1 ml of the solutions are placed in each well of a 24 well plate. Into each well a 1 cm fragments of 14G Teflon catheters are inserted and the plates incubated for 24 at 37 C to allow biofilm formation. Following incubation the biofilm catheter fragments are washed to remove non-biofilm cells and catheter pieces are implanted sub-cutinously the flanks of Balb/C mice. One test group (n=8, group #1) is implanted with the catheter that is pretreated with the peptide and the rest (n=24, groups #2-4) are implanted with the untreated catheter. The mouse are maintained and treated as follow: Group 1: No treatment; Group 2: No treatment; Group 3: Modified peptide #3 at local injection subcutaneous dose of 0.1 mg/ml twice a day. The mice are maintained for 5 days following which the catheters are removed and the biofilm biomass on each catheter was evaluated by viable counts.

1. An inhibitor of a bacterial biofilm formation comprising at least one of:
(a) at least one amino acid sequence derived from the N′ loop extension of the periplasmic subunit of a bacterial Phosphate Specific Transfer system (PstS), any ortholog or of any fragment thereof, or any nucleic acid sequence encoding the same; and
(b) at least one compound that specifically binds to said N′ loop extension of PstS.

2. The inhibitor according to claim 1, wherein said N′ loop extension comprises residues 25 to 39 of P. aeruginosa PstS, as denoted by SEQ ID NO. 2 or any derivative/s or fragment/s thereof.

3. The inhibitor according to claim 2, wherein said inhibitor is at least one isolated and purified peptide derived from the N′ loop extension of the P. aeruginosa PstS, said peptide comprises the amino acid sequence Xaa₍ₙ₎₋Ala-Ile-Asp-Pro-Ala-Leu-Pro-Glu-Xaa₍ₙ₎ as denoted by SEQ ID NO. 23 or any fragment/s thereof, wherein Xaa is any amino acid and n is zero or an integer of from 1 to 10.

4. The inhibitor according to claim 3, wherein said inhibitor is at least one isolated and purified peptide comprising the amino acid sequence Ala-Ile-Asp-Pro-Ala-Leu-Pro-Glu as denoted by SEQ ID NO. 27 or any fragment/s, enantiomer/s or derivative/s thereof.

5. The inhibitor according to claim 4, wherein at least one amino acid residue of an enantiomer of a peptide comprising the amino acid sequence as denoted by SEQ ID NO. 27, is a D-enantiomer.

6. The inhibitor according to claim 5, wherein the N-terminal Ala and the C terminal Glu of said peptide are D-enantiomers, said peptide comprises the amino acid sequence as denoted by SEQ ID NO. 56.

7. The inhibitor according to claim 2, wherein said inhibitor is at least one isolated and purified peptide derived from the N′ loop extension of the P. aeruginosa PstS, said peptide comprises the amino acid sequence of at least one of:
(a) Ala-Ile-Asp-Pro-Ala-Leu-Pro-Glu-Xaa₍ₙ₎ as denoted by SEQ ID NO. 24 or any fragment/s, enantiomer/s or derivative/s thereof, wherein Xaa is any amino acid and n is zero or an integer of from 1 to 10;
(b) Ala-Ile-Asp-Pro-Ala-Leu-Pro-Glu-Tyr-Gln-Lys-Ala-Ser as denoted by SEQ ID NO. 26 or any fragment/s, enantiomer/s or derivative/s thereof;
(c) Ala-Ile-Asp-Pro-Ala-Leu-Pro-Glu-Tyr-Gln-Lys-Ala-Ser-Gly-Val-Ser-Gly as denoted by SEQ ID NO. 25 or any fragment/s, enantiomer/s or derivative/s thereof;
(d) Pro-Glu-Tyr-Gln-Lys as denoted by SEQ ID NO. 28 or any fragment/s, enantiomer/s or derivative/s thereof;
(e) Glu-Tyr-Gln-Lys, as denoted by SEQ ID NO. 29 or any fragment/s, enantiomer/s or derivative/s thereof; and
(f) Tyr-Gln-Lys-Ala-Ser-Gly-Val-Ser-Gly as denoted by SEQ ID NO. 30 or any fragment/s, enantiomer/s or derivative/s thereof.

8. The inhibitor according to claim 1, wherein said inhibitor is at least one isolated and purified antibody that specifically recognizes and binds the N′ loop extension of PstS or any fragment thereof.

9. An isolated and purified peptide comprising the amino acid sequence of the N′ loop extension of P. aeruginosa PstS or any derivative/s, enantiomer/s and fragment/s thereof, said N′ loop extension comprises residues 25 to 39 of P. aeruginosa PstS, as denoted by SEQ ID NO. 2 or any fragment thereof.

10. The peptide according to claim 9, wherein said peptide comprises the amino acid sequence Ala-Ile-Asp-Pro-Ala-Leu-Pro-Glu as denoted by SEQ ID NO. 27 or any fragment/s, enantiomer/s or derivative/s thereof.

11. The peptide according to claim 10, wherein at least one amino acid residue of an enantiomer of a peptide comprising the amino acid sequence as denoted by SEQ ID NO. 27, is a D-enantiomer.

12. The peptide according to claim 11, wherein the N-terminal Ala and the C terminal Glu of said peptide are D-enantiomers, said peptide comprises the amino acid sequence as denoted by SEQ ID NO. 56.

13. The peptide according to claim 9, wherein said peptide comprises the amino acid sequence of anyone of:
(a) Ala-Ile-Asp-Pro-Ala-Leu-Pro-Glu-Xaa₍ₙ₎ as denoted by SEQ ID NO. 24 or any fragment/s, enantiomer/s or derivative/s thereof, wherein Xaa is any amino acid and n is zero or an integer of from 1 to 10;
(b) Ala-Ile-Asp-Pro-Ala-Leu-Pro-Glu-Tyr-Gln-Lys-Ala-Ser as denoted by SEQ ID NO. 26 or any fragment/s, enantiomer/s or derivative/s thereof;
(c) Ala-Ile-Asp-Pro-Ala-Leu-Pro-Glu-Tyr-Gln-Lys-Ala-Ser-Gly-Val-Ser-Gly as denoted by SEQ ID NO. 25 or any fragment/s, enantiomer/s or derivative/s thereof;
(d) Pro-Glu-Tyr-Gln-Lys as denoted by SEQ ID NO. 28 or any fragment/s, enantiomer/s or derivative/s thereof;
(e) Glu-Tyr-Gln-Lys, as denoted by SEQ ID NO. 29 or any fragment/s, enantiomer/s or derivative/s thereof; and
(f) Tyr-Gln-Lys-Ala-Ser-Gly-Val-Ser-Gly as denoted by SEQ ID NO. 30 or any fragment/s, enantiomer/s or derivative/s thereof.

14. A composition comprising as an active ingredient at least one inhibitor of a bacterial biofilm formation, wherein said inhibitor is as defined in claim 1, said composition optionally further comprises at least one pharmaceutically acceptable carriers, excipients, auxiliaries, and/or diluents.

15. The composition according to claim 14 comprising at least one of:
(a) at least one isolated and purified peptide comprising the amino acid sequence of any one of SEQ ID NO. 27, 56, 25, 26, 28, 29, 30, 23 or 24, or of any fragment or derivatives thereof;
(b) at least one isolated and purified nucleic acid sequence encoding the N′ loop extension of P. aeruginosa PstS or any fragment thereof, or any expression vector comprising said nucleic acid sequence;
(c) at least one isolated and purified antibody that specifically recognizes and binds the N′ loop extension of PstS or any fragment thereof; and
(d) any combinations of (a), (b) and (c).

16. A method for inhibiting, reducing or eliminating bacterial biofilm formation in at least one of, a subject, a surface, and a substance, the method comprising administering to said subject or contacting, applying or dispensing to said surface or substance an effective amount of at least one inhibitor of a bacterial biofilm formation or any composition comprising the same, wherein said inhibitor comprises at least one of:
(a) at least one amino acid sequence derived from the N′ loop extension of PstS, any ortholog, or of any fragment thereof, or any nucleic acid sequence encoding the same; and
(b) at least one compound that specifically binds to said N′ loop extension of PstS.

17. The method according to claim 16, wherein said inhibitor comprises at least one of:
(a) at least one isolated and purified peptide comprising the amino acid sequence of any one of SEQ ID NO. 27, 56, 25, 26, 28, 29, 30, 23 or 24, or of any fragment or derivatives thereof;
(b) at least one isolated and purified nucleic acid sequence encoding the N′ loop extension of P. aeruginosa PstS, any ortholog or any fragment thereof, or any expression vector comprising said nucleic acid sequence;
(c) at least one isolated and purified antibody that specifically recognizes and binds the N′ loop extension of PstS or any fragment thereof; and
(d) any combinations of (a), (b) and (c).

18. A method for treating, preventing, ameliorating, reducing or delaying the onset of an infectious clinical condition in a subject in need thereof, the method comprising the step of administrating to said subject a therapeutically effective amount of at least one inhibitor of a bacterial biofilm formation or of any composition comprising the same, wherein said inhibitor comprises at least one of:
(a) at least one amino acid sequence derived from the N′ loop extension of PstS, any ortholog, or of any fragment thereof, or any nucleic acid sequence encoding the same; and
(b) at least one compound that specifically binds to said N′ loop extension of PstS.

19. The method according to claim 18, wherein said inhibitor comprises at least one of:
(a) at least one isolated and purified peptide comprising the amino acid sequence of any one of SEQ ID NO. 27, 56, 25, 26, 28, 29, 30, 23 or 24, or of any fragment or derivatives thereof;
(b) at least one isolated and purified nucleic acid sequence encoding the N′ loop extension of P. aeruginosa PstS or any fragment thereof, or any expression vector comprising said nucleic acid sequence;
(c) at least one isolated and purified antibody that specifically recognizes and binds the N′ loop extension of PstS or any fragment thereof; and
(d) any combinations of (a), (b) and (c).

20. The method according to claim 18, wherein said infectious clinical condition is caused by P. aeruginosa.
